(19) United States
(12) Patent Application Publication
(10) Pub. No.: US 2024/0206475 A1
(43) Pub. Date: Jun. 27, 2024

(54) COMPOSITIONS AND METHODS FOR IMPROVING PLANT HEALTH AND CONTROLLING PLANT DISEASE

(71) Applicant: AgBiome, Inc., Durham, NC (US)

(72) Inventors: Vincent James Spadafora, Yardley, PA (US); Scott Louis Walker, Cary, NC (US)

(73) Assignee: AgBiome, Inc., Durham, NC (US)

(21) Appl. No.: 18/555,952

(22) PCT Filed: Apr. 19, 2022
(86) PCT No.: PCT/US2022/025345

§ 371 Date: Oct. 18, 2023

Related U.S. Application Data
(60) Provisional application No. 63/176,698, filed on Apr. 19, 2021.
(60) Provisional application No. 63/176,704, filed on Apr. 19, 2021.
(60) Provisional application No. 63/192,860, filed on May 25, 2021.
(60) Provisional application No. 63/254,795, filed on Oct. 12, 2021.

(51) Int. Cl. A01N 63/22; A01N 63/27; A01P 1/00; A01P 3/00; A01P 21/00

(52) U.S. Cl. CPC A01N 63/22; A01N 63/27; A01P 1/00; A01P 3/00; A01P 21/00

(57) ABSTRACT

Compositions and methods for controlling a plant pest or for treating or preventing plant disease are provided. Such compositions and methods comprise a bacterial strain that controls one or more plant pests or that improves at least one agronomic trait of interest in a plant. The bacterial strain can be used as an inoculant for plants. Methods for controlling a plant pest, for growing a plant susceptible to a plant disease, and for controlling plant disease on a plant susceptible to the plant disease are provided. Methods for improving at least one agronomic trait of interest in a plant are also provided.

### CROSS-REFERENCE TO RELATED APPLICATIONS

This application claims priority to U.S. Provisional Application Nos. 63/176,704, filed Apr. 19, 2021, 63/176,698, filed Apr. 19, 2021, 63/192,860, filed May 25, 2021, and 63/254,795, filed Oct. 12, 2021, each of which is incorporated by reference herein in its entirety.

### FIELD OF THE INVENTION

The invention relates to a bacterial strain and variants thereof and populations for controlling plant pests and disease and/or improving an agronomic trait of interest in a plant.

### BACKGROUND

Plant diseases and plant pests are responsible for significant agricultural losses. Effects can range from mild symptoms to catastrophic plant damage, which can lead to major economic and social consequences. Methods are needed to effectively control plant diseases and the pathogens that cause them and to effectively control plant pests.

### SUMMARY

Compositions and methods for controlling plant diseases and plant pests and/or for improving at least one agronomic trait of interest in a plant are provided. Such compositions and methods comprise a population of biocontrol agents or bacterial strains that control one or more pathogens that cause plant disease, one or more plant pests, and/or improve at least one agronomic trait of interest. The biological agents or bacterial strains can be used as an inoculant for plants. Methods for growing a plant susceptible to plant disease and methods and compositions for controlling plant disease and plant pests are also provided. Further provided are methods and compositions of increasing disease resistance and pest resistance in plants. Methods and compositions for improving plant health and/or improving at least one agronomic trait of interest are also provided.

### BRIEF DESCRIPTION OF THE FIGURES

FIG. 1 provides results from studies wherein harvested sweet cherries were treated with AIP61892 or AIP1620 pre-inoculation (FIG. 1A) or post-inoculation (FIG. 1B) with M. fructicola or B. cinerea.

FIG. 2 provides results from studies wherein harvested Bing cherries were treated with AIP61892 or AIP1620 after wound-inoculation with M. fructicola (FIG. 2A), B. cinerea (FIG. 2B), or R. stolonifera (FIG. 2C).

### DETAILED DESCRIPTION

### I. Overview

Compositions and methods for improving at least one agronomic trait of interest and/or improving plant health and/or for controlling one or more plant diseases and/or plant pests are provided. A biological agent, biocontrol agent, bacterial strain, modified bacterial strain, modified biological agent, or modified biocontrol agent or active variant therof, and/or a composition derived therefrom are used herein to describe a microorganism that is used to control plant pests, disease-causing plant pathogens and/or improve at least one agronomic trait of interest and/or improve plant health. The biocontrol agent can be used alone or in combination with another biocontrol agent or another pesticide, biocide, fungicide, bactericide, nematicide, insecticide or herbicide known in the art or disclosed herein. In some embodiments, the combination (applied simultaneously or sequentially) of at least one copper compound with AIP61892 or a variant thereof produce at least an additive effect, and in particular embodiments a synergistic effect, that allows for use of a lower amount of the biocontrol agent and/or copper compound than the suggested or commonly used amount for application. In this manner, lower amounts of biocides can be added to crops or plants in order to increase disease control, reduce chemical residues, reduce pathogen resistance, and increase product usage base acres in multiple crops for the bacterial strain and synthetic fungicide. Thus, in some embodiments, lower rates of the biocontrol agent and the copper compound can provide a synergistic effect (i.e., greater than additive or superadditive). In other embodiments, the combination (applied simultaneously or sequentially) of AIP61892 or a variant thereof and AIP1620 or a variant thereof produce at least an additive effect, and in particular embodiments a synergistic effect, that allows for use of a lower amount of one or both of the biological agents than the suggested or commonly used amount for application. Thus, in some embodiments, lower rates of one or both of the biocontrol agent can provide a synergistic effect. Moreover, in some embodiments, the combination (applied simultaneously or sequentially) of AIP61892 or a variant thereof and AIP1620 or a variant thereof produce a synergistic effect on the control of plant pests or disease-causing plant pathogens.

### II. Bacterial Strains

The biocontrol agent or bacterial strain AIP61892 or a variant of any thereof can be used to control one or more plant pest, one or more plant disease, and/or improve at least one agronomic trait of interest and/or improve plant health. Cell populations comprising AIP61892 are provided, as well as populations of spores derived from this strain, or any preparation thereof. The AIP61892 bacterial strains and/or the pesticidal compositions provided herein comprise as an active ingredient a cell population comprising or an active variant thereof.

Bacterial strain AIP61892 is described in WO 2017/040273, incorporated by reference herein, and was deposited with the Patent Depository of the National Center for Agricultural Utilization Research Agricultural Research Service, U.S. Department of Agriculture, 1815 North University Street, Peoria, Illinois 61604 U.S.A. on Aug. 6, 2015 and assigned NRRL No. B-67089. The deposit will be maintained under the terms of the Budapest Treaty on the International Recognition of the Deposit of Microorganisms for the Purposes of Patent Procedure. The deposit was made merely as a convenience for those of skill in the art and is not an admission that a deposit is required under 35 U.S.C. § 112.

Compositions and methods are provided herein comprising AIP61892 or a variant thereof combined with bacterial strain AIP1620 or a variant thereof. AIP1620 is described in US 2015/0218568, incorporated by reference in its entirety herein. AIP1620 was deposited with the Patent Depository of the National Center for Agricultural Utilization Research Agricultural Research Service, U.S. Department of Agriculture, 1815 North University Street, Peoria, Illinois 61604 U.S.A. on Jan. 31, 2014 and assigned NRRL No. B-50897. The deposit will be maintained under the terms of the Budapest Treaty on the International Recognition of the Deposit of Microorganisms for the Purposes of Patent Procedure. This deposit was made merely as a convenience for those of skill in the art and not an admission that a deposit is required under 35 U.S.C. § 112.

The term “isolated” encompasses a bacterium, spore, or other entity or substance, that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated.

As used herein, a substance is “pure” if it is substantially free of other components. The terms “purify,” “purifying” and “purified” z refer to a bacterium, spore, or other material that has been separated from at least some of the components with which it was associated either when initially produced or generated (e.g., whether in nature or in an experimental setting), or during any time after its initial production. A bacterium or spore or a bacterial population or a spore population may be considered purified if it is isolated at or after production, such as from a material or environment containing the bacterium or bacterial population or spore, and a purified bacterium or bacterial population or spore may contain other materials up to about 10%, about 20%, about 30%, about 40%, about 50%, about 60%, about 70%, about 80%, about 90%, or above about 90% and still be considered purified. In some embodiments, purified bacteria or spores and bacterial populations or spore populations are more than about 80%, about 85%, about 90%, about 91%, about 92%, about 93%, about 94%, about 95%, about 96%, about 97%, about 98%, about 99%, or more than about 99% pure. In specific embodiments, a culture of bacteria contains no other bacterial species in quantities to be detected by normal bacteriological techniques.

In some embodiments, the compositions of the invention comprise substantially pure cultures of bacterial strain AIP61892. The compositions of the invention also provide progeny of substantially pure cultures of bacterial strain AIP61892, wherein the culture has all of the physiological and morphological characteristics of AIP61892.

By “population” is intended a group or collection that comprises two or more individuals (i.e., 10, 100, 1,000, 10,000, 1×10⁶, 1×10⁷, or 1×10⁸ or greater) of a given bacterial strain. Various compositions are provided herein that comprise a population of at least one bacterial strain or a mixed population of individual poplations from more than one bacterial strain. In specific embodiments, the population of bacterial strain AIP61892 or AIP1620, or an active variant thereof, or spores or forespores or a combination of cells, forespores and/or spores, and/or a composition derived from AIP61892 or AIP1620 or an active variant thereof comprises a concentration of at least about 10³ CFU/ml to about 10⁵ CFU/ml, 10³ CFU/ml to about 10⁴ CFU/ml, 10³ CFU/ml to about 10⁶ CFU/ml, 10⁴ CFU/ml to about 10⁸ CFU/ml, 10⁵ CFU/ml to about 10” CFU/ml, about 10⁵ CFU/ml to about 10¹⁰ CFU/ml, about 10⁵ CFU/ml to about 10¹² CFU/ml, about 10⁵ CFU/ml to about 10⁶ CFU/ml, about 10⁶ CFU/ml to about 10⁷ CFU/ml, about 10⁷ CFU/ml to about 10⁸ CFU/ml, about 10⁸ CFU/ml to about 10⁹ CFU/ml, about 10⁹ CFU/ml to about 10¹⁰ CFU/ml, about 10¹⁰ CFU/ml to about 10¹¹ CFU/ml, about 10¹¹ CFU/ml to about 10¹² CFU/ml. In other embodiments, the concentration of the AIP61892 or AIP1620 bacterial strain or an active variant thereof and/or a composition derived therefrom comprises at least about 10⁵ CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, or at least about 10¹² CFU/ml. In particular embodiments, the population of AIP61892 or AIP1620 or an active variant thereof comprises a concentration of at least about 10³ CFU/g to about 10⁴, 10³ CFU/g to about 10⁵ CFU/g, CFU/g, 10³ CFU/g to about 10⁶ CFU/g, 10⁴ CFU/g to about 10⁸ CFU/g, 10⁵ CFU/g to about 10¹¹ CFU/g, about 10⁵ CFU/g to about 10¹⁰ CFU/g, about 10⁵ CFU/g to about 10¹² CFU/g, about 10⁵ CFU/g to about 10⁶ CFU/g, about 10⁶ CFU/g to about 10⁷ CFU/g, about 10⁷ CFU/g to about 10⁸ CFU/g, about 10⁸ CFU/g to about 10⁹ CFU/g, about 10⁹ CFU/g to about 10¹⁰ CFU/g, about 10¹⁰ CFU/g to about 10¹¹ CFU/g, about 10¹¹ CFU/g to about 10¹² CFU/g. In other embodiments, the concentration of the AIP61892 or AIP1620 bacterial strain or an active variant thereof comprises at least about 10² CFU/g, at least about 10³ CFU/g, at least about 10⁴ CFU/g, at least about 10⁵ CFU/g, at least about 10⁶ CFU/g, at least about 10⁷ CFU/g, at least about 10⁸ CFU/g, at least about 10⁹ CFU/g, at least about 10¹⁰ CFU/g, at least about 10¹¹ CFU/g, or at least about 10¹² CFU/g. The bacterial concentration of a given solid or liquid composition or formulation can be expressed in CFU/g or CFU/mL, respectively, or expressed as activity or viability using any methods described herein. For example, a measure bacterial viability that is equivalent to CFU can be expressed in terms of cells/g or cells/mL if using epifluorescent measurements or a measure of activity can be expressed as pg of a metabolite, such as pyrrolnitrin, per g of bacteria when using metabolite measurements as a reporter metabolite, such as for example using pyrrolnitrin as a reporter metabolite.

A “spore” refers to at least one dormant (at application) but viable reproductive unit of a bacterial species. Non-limiting methods by which spores are formed from AIP61892 (or variants thereof) are disclosed elsewhere herein. It is further recognized the populations disclosed herein can comprise a combination of vegetative cells and forespores (cells in an intermediate stage of spore formation); a combination of forespores and spores; or a combination of forespores, vegetative cells and/or spores.

As used herein, “derived from” means directly isolated or obtained from a particular source or alternatively having identifying characteristics of a substance or organism isolated or obtained from a particular source. In the event that the “source” is an organism, “derived from” means that it may be isolated or obtained from the organism itself or a culture broth, suspension, or medium used to culture or grow said organism. A compound or composition “derived from” or “obtainable from” means that the compound or composition may be isolated from or produced by a cell culture or a whole cell broth, or a suspension, filtrate, supernatant, fraction, or extract derived from a cell culture or a whole cell broth.

As used herein, “whole broth culture” or “whole cell broth” refers to a liquid culture containing both cells and media. If bacteria are grown on a plate, the cells can be harvested in water or other liquid, whole culture. The terms “whole broth culture” and “whole cell broth” are used interchangeably.

As used herein, “supernatant” refers to the liquid remaining when cells grown in broth or are harvested in another liquid from an agar plate and are removed by centrifugation, filtration, sedimentation, or other means well known in the art. In some embodiments, the supernatant may be diluted with another composition, such as water, buffer, fresh media, and/or a formulation. The diluted supernatant is still considered a supernatant of the invention.

As used herein, “filtrate” refers to liquid from a whole broth culture that has passed through a membrane. The filtrate may comprise a concentrated amount of an effective compound or metabolite compared to the concentration of the effective compound or metabolite in the whole broth culture or supernatant. As used herein, “extract” refers to liquid substance removed from cells by a solvent (water, detergent, buffer, and/or organic solvent, for example) and separated from the cells by centrifugation, filtration, or other method known in the art. The extract may comprise a concentrated amount of an effective compound or metabolite compared to the concentration of the effective compound or metabolite in the cells prior to extraction. Alternatively, the filtrate or extract may then be diluted with another composition, such as water, buffer, fresh media, and/or a formulation. Such diluted filtrates or extracts are still considered filtrates and extracts of the invention.

As used herein, “metabolite” refers to a compound, substance, or byproduct of fermentation of a bacterial strain. An effective compound or metabolite is a compound present in the supernatant, whole cell broth, or bacterial strain which may improve any agronomic trait of interest of a plant, or which controls a plant pest or a plant pathogen that causes a plant disease, when applied to a plant of interest at an effective amount.

In some embodiments, a composition of the invention comprises a filtrate or extract derived from fermentation of the AIP61892 bacterial strain or an active variant thereof (and in some embodiments, the AIP1620 bacterial strain or an active variant thereof), wherein said composition comprises a concentrated amount of an effective compound or metabolite compared to the amount in a whole cell broth or supernatant of said bacterial strain. In other embodiments, a composition of the invention comprises a diluted filtrate, diluted extract, or diluted supernatant derived from the fermentation of the AIP61892 bacterial strain or an active variant thereof, wherein said composition comprises a diluted amount of the effective compound or metabolite compared to the amount whole cell broth or undiluted supernatant of said bacterial strain. The diluted filtrate, diluted extract, or diluted supernatant may still comprise an effective amount of the effective compound or metabolite.

The compositions and methods described herein comprise or are derived from AIP61892 or an active variant thereof, or a spore or a forespore or a combination of cells, forespores or/and spores, from AIP61892 or an active variant thereof. Methods also comprise cultivating bacterial strain AIP61892 or an active variant thereof. In some embodiments, bacterial strain AIP61892 or an active variant thereof is cultivated and compounds and/or compositions are obtained by isolating these compounds and/or compositions from the culture of AIP61892 or an active variant thereof.

In some embodiments, bacterial strain AIP61892 or an active variant thereof is cultivated in nutrient medium using methods known in the art. The bacterial strain can be cultivated by shake flask cultivation or by small scale or large scale fermentation (including but not limited to continuous, batch, fed-batch, or solid state fermentation) in laboratory or industrial fermenters performed in a suitable medium and under conditions allowing for bacterial cell growth. The cultivation can take place in suitable nutrient medium comprising carbon and nitrogen sources and inorganic salts, using prodedures known in the art. Suitable media are available from commercial sources or are prepared according to publications well-known in the art.

Following cultivation, compounds, metabolites, and/or compositions can be extracted from the culture broth. The extract can be fractionated by chromatography. The extract can be further purified using methods well-known in the art. The extract can also be diluted using methods well-known in the art.

The compositions comprising bacterial strain AIP61892 or an active variant thereof, or a spore or a forespore or a combination of cells, forespores and/or spores, and/or a composition derived from AIP61892 or an active variant thereof can further comprise an agriculturally acceptable carrier. The term “agriculturally acceptable carrier” is intended to include any material that facilitates application of a composition to the intended subject (i.e, a plant or plant part susceptible to a plant pest or plant disease of interest (i.e., powdery mildew), or any other pest or disease disclosed herein or a plant or plant part for improving an agronomic trait of interest. Carriers used in compositions for application to plants and plant parts are preferably non-phytotoxic or only mildly phytotoxic. A suitable carrier may be a solid, liquid or gas depending on the desired formulation. In one embodiment, carriers include polar or non-polar liquid carriers such as water, mineral oils and vegetable oils. Additional carriers are disclosed elsewhere herein.

### A. Active Variants of a Bacterial Strain

Further provided are active variants of AIP61892. Some embodiments also comprise active variants of AIP1620. Such variants will retain the ability to control one or more plant diseases (i.e., reduce disease severity and/or reduce disease development), and/or control one or more plant pests (e.g., bacterial pests, fungal and fungal-like pests, nematode pests, insect pests). In some embodiments, variants will retain the ability to control one or more fungal and/or bacterial plant diseases and/or one or more fungal and/or bacterial pathogens. In some embodiments, variants will retain all of the physiological and morphological characteristics of the parent bacterial strain disclosed herein. Active variants of the various bacterial strains provided herein include, for example, any isolate or mutant of AIP61892 which retains the ability to control plant diseases. In some embodiments, active variants of AIP61892 will retain the ability to synergistically control one or more plant pests or pathogens in combination with a copper compound or AIP1620 or a variant thereof.

As used herein, “pesticidal activity” refers to activity against one or more pests, including fungi, fungal-like pathogens (e.g., Oomycetes, plasmodiophorids, and the Phytomyxea), bacteria, insects, nematodes, viruses, viroids, protozoan pathogens, and the like, such that the pest is killed or controlled. In some embodiments, variants will retain the ability to control one or more insect pests or nematode pests. In particular embodiments, variants will retain the ability to control fungal and fungal-like pests, including Oomycetes such as Pythium, Phytophthora, and downy mildews. As used herein downy mildews include but are not limited to pathogenic species of the genera Peronospora, Pseudoperonospora, Bremia, Plasmopara, and Basidiophora.

The term “mutant” refers to a variant of the parental stran as well as methods for obtaining a mutant or variant in which the pesticidal activity is greater than that expressed by the parental strain. The “parent strain” is the original strain before mutagenesis. To obtain such mutants the parental strain may be treated with a chemical such as N-methyl-N′-nitro-N-nitrosoguanidine, ethylmethanesulfone (EMS), or by irradiation using gamma, x-ray, or UV-irradiation, or by other means well known in the art.

In some embodiments, the mutant may be the result of a spontaneous mutation which generates a phenotype. Such a mutant may be derived from an AIP61892 population when grown continuously in liquid culture. The spontaneous mutation may be a naturally occurring mutation or an induced mutation. In other embodiments, the spontaneous mutation may be derived from an AIP61892 population when grown continuously on a solid media, such as an agar plate. These spontaneous mutants are considers derivatives of the bacterial strain of the invention. Said derivatives may be derived from the deposited strain. In some embodiments, derivatives retain all of the physiological and morphological characteristics of the bacterial strain of the invention.

In some embodiments, the variant or derivative contains a mutation in at least one gene, relative to the deposited strain. The gene(s) may have a role in, for example, biofilm formation, motility, chemotaxis, extracellular secretion, transport (for example ABC transporter proteins), stress responses, volatiles, transcription (for example alternative sigma factors and global transcription regulators), root colonization, ability to stimulate induced systemic resistance in a plant, and/or secondary metabolism including synthesis of lipopeptides, polyketides, macromolecular hydrolases (for example proteases and/or carbohydrases), and/or antimicrobial compounds including antibiotics. Secondary metabolism refers to both non-ribosomal and ribosomal synthesis of antimicrobial compounds, including cyclic lipopeptides, polyketides, iturins, bacteriocins (for example plantazolicin and amylocyclicin) and dipeptides (for example bacilysin).

An example of a variant is a cell of bacterial strain disclosed herein, wherein the cell further comprises a mutation in the swrA gene that results in loss of function. The swrA mutation, which affects biofilm formation (Kearns et al., Molecular Microbiology (2011) 52(2): 357-369) may result in a variant of a strain of the invention which has enhanced ability to control a plant pest or improve an agronomic trait of interest of a plant. Other genes that are involved in biofilm formation, such as sfp, epsC, degQ, and a plasmid gene called rapP (see for example, McLoon et al., J of Bacteriology, (2011) 193(8): 2027-2034), may also be mutated in an active variant of a bacterial strain of the invention.

In specific embodiments, the bacterial strain is compatible with a biocide. A biocide is a chemical substance that can exert a controlling effect on an organism by chemical or biological means. Biocides include pesticides, such as fungicides; herbicides; insecticides, other crop protection chemicals, and the like. Such compounds are discussed in detail elsewhere herein. A bacterial strain is compatible with a biocide when the bacterial strain is able to survive and/or reproduce in the presence of an effective amount of a biocide of interest. In instances where the bacterial strain is not compatible for a biocide of interest, if desired, methods can be undertaken to modify the bacterial strain to impart the compatibility of interest. Such methods to produce modified bacterial strains include both selection techniques and/or transformation techniques.

By “modified bacterial strain” is intended a population wherein the strain has been modified (by selection and/or transformation) to have one or more additional traits of interest. In some embodiments the modified bacterial strain is an active variant of AIP61892. In specific embodiments, the modified bacterial strain is compatible with a biocide of interest, including but not limited to, resistance to a herbicide, fungicide, pesticide, or other crop protection chemical. The modified biocide-resistant strains have the same identification characteristics as the original sensitive strain except they are significantly more resistant to the particular herbicide, fungicide, pesticide, or other crop protection chemical. Their identification is readily possible by comparison with characteristics of the known sensitive strain. Thus, isolated populations of modified bacterial strains are provided.

An increase in resistance to a biocide (i.e., for example, a herbicide, fungicide, pesticide, or other crop protection chemical resistance) refers to the ability of an organism (i.e., bacterial cell or spore) to survive and reproduce following exposure to a dose of the biocide (e.g, herbicide, fungicide, pesticide, or other crop protection chemical) that would normally be lethal to the unmodified organism or would substantially reduce growth of the unmodified organism. In specific embodiments, the increase in resistance to a biocide is demonstrated in the presence of an agriculturally effective amount of the biocide.

In such instances, the modified bacterial strain having resistance to one or more biocides is useful for enhancing the competitiveness of bacterial strains particularly over other microbial agents which are not resistant to herbicides, fungicides, pesticides, or other crop protection chemicals. Therefore, compositions provided herein include selected or engineered bacterial strains and modified populations of bacterial strains. These bacterial strains or modified bacterial strains can be used as an inoculant for plants. They can also be applied as a spray application directly to the aerial parts of plants, and can be mixed with the herbicide or other chemical to which they have been modified to become tolerant.

Thus, active variants of AIP61892 include a modified strain, such that the active variant controls a plant disease and further is able to grow in the presence of at least one biocide.

Recombinant bacterial strains having resistance to an herbicide, fungicide, pesticide, or other crop protection chemical can be made through genetic engineering techniques and such engineered or recombinant bacterial strains may be grown to produce a modified population of bacterial strains. A recombinant bacterial strain is produced by introducing polynucleotides into the bacterial host cell by transformation. Methods for transforming microorganisms are known and available in the art. See, generally, Hanahan, D. (1983) Studies on transformation of Escherichia coli with plasmids J. Mol. Biol. 166, 557-77; Seidman, C. E. (1994) In: Current Protocols in Molecular Biology, Ausubel, F. M. et al. eds., John Wiley and Sons, NY; Choi et al. (2006) J. Microbiol. Methods 64:391-397; Wang et al. 2010. J. Chem. Technol. Biotechnol. 85:775-778. Transformation may occur by natural uptake of naked DNA by competent cells from their environment in the laboratory. Alternatively, cells can be made competent by exposure to divalent cations under cold conditions, by electroporation, by exposure to polyethylene glycol, by treatment with fibrous nanoparticles, or other methods well known in the art.

Herbicide resistance genes useful in transforming a bacterial strain include, but are not limited to, fumonisin detoxification genes (U.S. Pat. No. 5,792,931); acetolactate synthase (ALS) mutants that lead to herbicide resistance, in particular the sulfonylurea-type herbicides, such as the S4 and/or Hra mutations; inhibitors of glutamine synthase such as phosphinothricin or basta (e.g., bar gene); and glyphosate resistance (EPSPS gene); gluphosinate, and HPPD resistance (WO 96/38576, U.S. Pat. Nos. 6,758,044; 7,250,561; 7,935,869; and 8,124,846), or other such genes known in the art. The disclosures of WO 96/38576, U.S. Pat. Nos. 5,792,931, 6,758,044; 7,250,561; 7,935,869; and 8,124,846 are herein incorporated by reference. The bar gene encodes resistance to the herbicide basta, the nptII gene encodes resistance to the antibiotics kanamycin and geneticin, and the ALS-gene mutants encode resistance to the sulfonylurea herbicides including chlorsulfuron, metsulfuron, sulfometuron, nicosulfuron, rimsulfuron, flazasulfuron, sulfosulfuron, and triasulfuron, and the imadizolinone herbicides including imazethapyr, imazaquin, imazapyr, and imazamethabenz.

To identify and produce a modified population of bacterial strains through selection, the bacterial strains are grown in the presence of the herbicide, fungicide, pesticide, or other crop protection chemical as the selection pressure. Susceptible agents are killed while resistant agents survive to reproduce without competition. As the bacterial strains are grown in the presence of the herbicide, fungicide, pesticide, or other crop protection chemical, resistant bacterial strains successfully reproduce and become dominant in the population, becoming a modified population of bacterial strains. Methods for selecting resistant strains are known and include U.S. Pat. Nos. 4,306,027 and 4,094,097, herein incorporated by reference. The active variant of the bacterial strain comprising a modified population of bacterial strains will have the same identification characteristics as the original sensitive strain except they are significantly more tolerant to the particular herbicide, fungicide, pesticide, or other crop protection chemical. Thus, their identification is readily possible by comparison with characteristics of the known sensitive strain.

Further active variants of the various bacteria provide herein can be identified employing, for example, methods that determine the sequence identity relatedness between the 16S ribosomal RNA, methods to identify groups of derived and functionally identical or nearly identical strains include Multi-locus sequence typing (MLST), concatenated shared genes trees, Whole Genome Alignment (WGA), Average Nucleotide Identity, and MinHash (Mash) distance metric.

In one aspect, the active variants of a bacterial strain disclosed herein include strains that are closely related to said bacterial strain by employing the Bishop MLST method of organism classification as defined in Bishop et al. (2009) BMC Biology 7(1)1741-7007-7-3. Thus, in specific embodiments, an active variant of the bacterial strain AIP61892 includes a bacterial strain that falls within at least a 80%, 85%, 86%, 87%, 88%, 89%, 90%, 91%, 92%, 93%, 94%, 95%, 96%, 97%, 98%, 98.5%, 98.8%, 99%, 99.1%, 99.2%, 99.3%, 99.4%, 99.5%, 99.6%, 99.7%, 99.8%, or 99.9% sequence cut off employing the Bishop method of organism classification as set forth in Bishop et al. (2009) BMC Biology 7(1)1741-7007-7-3, which is herein incorporated by reference in its entirety. Active variants of the bacteria identified by such methods will retain the ability to improve at least one agronomic trait when applied in an effective amount to a plant, plant part, or an area of cultivation, including for example, reducing plant disease severity, reducing plant disease development, increasing plant resistance, increasing plant health, and/or improving an agronomic trait of interest in a plant.

In another aspect, the active variant of the bacterial strain(s) disclosed herein include strains that are closely related to any of the disclosed strains on the basis of the Average Nucleotide Identity (ANI) method of organism classification. ANI (see, for example, Konstantinidis, K. T., et al., (2005) PNAS USA 102(7):2567-72; and Richter, M., et al., (2009) PNAS 106(45):19126-31) and variants (see, for example, Varghese, N.J., et al., Nucleic Acids Research (Jul. 6, 2015): gkv657) are based on summarizing the average nucleotides shared between the genomes of strains that align in WGAs. Thus, in specific embodiments, an active variant of bacterial strain AIP61892 disclosed herein includes a bacterial stain that falls within at least a 90%, 95%, 96%, 97%, 97.5%, 98%, 98.5%, 98.8%, 99%, 99.5%, or 99.8% sequence cut off employing the ANI method of organism classification as set forth in Konstantinidis, K. T., et al., (2005) PNAS USA 102(7):2567-72, which is herein incorporated by reference in its entirety. Active variants of the bacteria identified by such methods will retain the ability to improve at least one agronomic trait when applied in an effective amount to a plant, plant part, or an area of cultivation, including for example, reducing plant disease severity and/or, reducing plant disease development, and/or increasing plant resistance to a pest.

In another aspect, the active variants of the isolated bacterial strains disclosed herein includes strains that are closely related on the basis of 16S rDNA sequence identity. See Stackebrandt E, et al., “Report of the ad hoc committee for the re-evaluation of the species definition in bacteriology,” Int J Syst Evol Microbiol. 52(3):1043-7 (2002) regarding use of 16S rDNA sequence identity for determining relatedness in bacteria. In an embodiment, the at least one strain is at least 95% identical to any of the above strains on the basis of 16S rDNA sequence identity, at least 96% identical to any of the above strains on the basis of 16S rDNA sequence identity, at least 97% identical to any of the above strains on the basis of 16S rDNA sequence identity, at least 98% to any of the above strains on the basis of 16S rDNA sequence identity, at least 98.5% identical to any of the above strains on the basis of 16S rDNA sequence identity, at least 99% identical to any of the above strains on the basis of 16S rDNA sequence identity, at least 99.5% to any of the above strains on the basis of 16S rDNA sequence identity or at least 100% to any of the above strains on the basis of 16S rDNA sequence identity. Active variants of the bacteria identified by such methods will retain the ability to improve at least one agronomic trait when applied in an effective amount to a plant, plant part, or an area of cultivation, including for example, reducing plant disease severity, increasing plant resistance to a pest, and/or reducing plant disease development.

The MinHash (Mash) distance metric is a comparison method that defines thresholds for hierarchical classification of microorganisms at high resolution and requires few parameters and steps (Ondov et al. (2016) Genome Biology 17:132). The Mash distance estimates the mutation rate between two sequences directly from their MinHash sketches (Ondov et al. (2016) Genome Biology 17:132). Mash distance strongly corresponds to Average Nucleotide Identity method (ANI) for hierarchical classification (See, Konstantinidis, K. T. et al. (2005) PNAS USA 102(7):2567-72, herein incorporated by reference in its entirety). That is, an ANI of 97% is approximately equal to a Mash distance of 0.03, such that values put forth as useful classification thresholds in the ANI literature can be directly applied with the Mash distance.

Active variants of the bacterial strain AIP61892 include strains that are closely related on the basis of the Minhash (Mash) distance between complete genome DNA sequences. Thus, in specific embodiments, an active variant of a bacterial strain disclosed herein includes bacterial strains having a genome within a Mash distance of less than about 0.015 to the disclosed strains. In other embodiments, an active variant of a bacterial strain disclosed herein includes a distance metric of less than about 0.001, 0.0025, 0.005, 0.010, 0.015, 0.020, 0.025, or 0.030. A genome as it relates to the Mash distance includes both bacterial chromosomal DNA and bacterial plasmid DNA. In other embodiments, the active variant of a bacterial strain has a genome that is above a Mash distance threshold to the disclosed strains that is greater than dissimilarity caused by technical variance. In further instances, the active variant of a bacterial strain has a genome that is above a Mash distance threshold to the disclosed strains that is greater than dissimilarity caused by technical variance and has a Mash distance of less than about 0.015. In other instances, the active variant of a bacterial strain has a genome that is above a Mash distance threshold to the disclosed strains that is greater than dissimilarity caused by technical variance and has a Mash distance of less than about 0.001, 0.0025, 0.005, 0.010, 0.015, 0.020, 0.025, or 0.030.

As used herein, “above technical variation” means above the Mash distance between two strains caused by errors in the genome assemblies provided the genomes being compared were each DNA sequenced with at least 20× coverage with the Illumina HiSeq 2500 DNA sequencing technology and the genomes are at least 99% complete with evidence for contamination of less than 2%. While 20× coverage is an art recognized term, for clarity, an example of 20× coverage is as follows: for a genome size of 5 megabases (MB), 100 MB of DNA sequencing from the given genome is required to have 20× sequencing coverage on average at each position along the genome. There are many suitable collections of marker genes to use for genome completeness calculations including the sets found in Campbell et al. (2013) PNAS USA 110(14):5540-45, Dupont et al. (2012) ISMEJ 6:1625-1628, and the CheckM framework (Parks et al. (2015) Genome Research 25:1043-1055); each of these references is herein incorporated in their entirety. Contamination is defined as the percentage of typically single copy marker genes that are found in multiple copies in the given genome sequence (e.g. Parks et al. (2015) Genome Research 25:1043-1055); each of these references is herein incorporated in their entirety. Completeness and contamination are calculated using the same collection of marker genes. Unless otherwise stated, the set of collection markers employed in the completeness and contamination assay is the set forth in Campbell et al. (2013) PNAS USA 110(14):5540-45, herein incorporated by reference.

Exemplary steps to obtain a distance estimate between the genomes in question are as follows: (1) Genomes of sufficient quality for comparison must be produced. A genome of sufficient quality is defined as a genome assembly created with enough DNA sequence to amount to at least 20× genome coverage using Illumina HiSeq 2500 technology. The genome must be at least 99% complete with contamination of less than 2% to be compared to the claimed microbe's genome. (2) Genomes are to be compared using the Minhash workflow as demonstrated in Ondov et al. (2016) Genome Biology 17:132, herein incorporated by reference in its entirety. Unless otherwise stated, parameters employed are as follows: “sketch” size of 1000, and “k-mer length” of 21. (3) Confirm that the Mash distance between the 2 genomes is less than 0.001, 0.0025, 0.005, 0.010, 0.015, 0.020, 0.025, or 0.030. Using the parameters and methods stated above, a Mash distance of 0.015 between two genomes means the expected mutation rate is 0.015 mutations per homologous position. Active variants of the bacteria identified by such methods will retain the ability to improve at least one agronomic trait when applied in an effective amount to a plant, plant part, or an area of cultivation, including for example, reducing plant disease severity, reducing plant disease development, and/or increasing plant resistance to a pest.

### III. Formulations

The bacterial strains provided herein (i.e., AIP61892 or AIP1620 or an active variant of any thereof, or a spore or a forespore or a combination of cells, forespores, and/or spores, and/or a composition derived from AIP61892 or AIP1620 or an active variant thereof) can be formulated as a cell paste, wettable powders, a cell pellet, dusts, granules, a slurry, a dry powder, aqueous or oil based liquid products, and the like. Such formulations will comprise the bacteria provided herein or an active variant thereof, and/or a composition derived therefrom in addition to carriers and other agents. In some embodiments, the formulation will comprise AIP61892 or an active variant thereof and a copper compound. In some embodiments, the formulation will comprise AIP61892 and AIP1620 or an active variant of any thereof. The formulations can be used in a variety of methods as disclosed elsewhere herein.

The bacterial strain AIP61892 or active variants thereof can be formulated to include at least one or more of an extender, a solvent, spontaneity promoter, carrier, emulsifier, dispersant, frost protectant, thickener, and/or adjuvant. In some embodiments, the extender, solvent, spontaneity promoter, carrier, emulsifier, dispersant, frost protectant, thickener, and/or adjuvant is a non-natural or synthetic extender, a solvent, spontaneity promoters, carriers, emulsifiers, dispersants, frost protectants, thickeners, and/or adjuvants. In particular embodiments, the bacterial strains disclosed herein and the active variants thereof can be formulated to include at least one or more natural extender, a solvent, spontaneity promoter, carrier, emulsifier, dispersant, frost protectant, thickener, and/or adjuvant.

Examples of typical formulations include water-soluble liquids (SL), emulsifiable concentrates (EC), emulsions in water (EW), suspension concentrates (SC), suspo-emulsions (SE), flowable concentrates for seed treatment (FS), oil dispersions (OD), water-dispersible granules (WG), granules (GR), capsule concentrates (CS), water-dispersible granules (WG), granules (GR), block baits (BB), water-soluble granules (SG), and mixed formulations of CS and SC (ZC). In some embodiments, the formulation may be a waxy coating. These and other possible types of formulation are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations may comprise active agrochemical compounds other than one or more active compounds of the invention.

The formulations or application forms of the various bacterial strains or active variants thereof can comprise, but are not limited to, auxiliaries, such as extenders, solvents, spontaneity promoters, carriers, emulsifiers, dispersants, frost protectants, biocides, solid carriers, surfactants, thickeners and/or other auxiliaries, such as adjuvants. An adjuvant in this context is a component which enhances the biological effect of the formulation, without the component itself having a biological effect. Examples of adjuvants are agents which promote the retention, spreading, attachment to the leaf surface, or penetration.

Non-limiting extenders are, for example, water, polar and nonpolar organic chemical liquids, for example from the classes of the aromatic and non-aromatic hydrocarbons (such as paraffins, alkyl benzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide). If the extender used is water, it is also possible to employ, for example, organic solvents as auxiliary solvents. Essentially, non-limiting liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water. In principle it is possible to use any suitable solvent. Non-limiting solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

Non-limiting examples of suitable carriers include, for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs, and tobacco stalks.

Liquefied gaseous extenders or solvents may also be used. Non-limiting examples are those extenders or carriers which at standard temperature and under standard pressure are gaseous, examples being aerosol propellants, such as halogenated hydrocarbons, and also butane, propane, nitrogen and carbon dioxide. Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkylta urates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum, and zinc.

Stabilizers, such as low-temperature stabilizers, preservatives, antioxidants, light stabilizers or other agents which improve chemical and/or physical stability may also be present. Additionally present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur et al., 1997, Pesticide Science 51: 131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (15), or ammonium and/or phosphonium salts such as ammonium sulphate or diammonium hydrogen phosphate, for example.

The various compositions and formulations disclosed herein can comprise an amount of the bacterial strain AIP61892 or AIP1620 or an active variant thereof, or a spore or a forespore or a combination of cells, forespores or/and spores, and/or can comprise an amount of a composition derived from AIP61892 or AIP1620 or an active variant thereof. In certain embodiments, the viability of a composition comprising the bacterial strain AIP61892 or AIP1620 or an active variant thereof can be quantitated by measuring the number of colony forming units per gram or per ml of the composition. In particular embodiments, the composition comprises a concentration of the bacterial strain of at least about 10⁴ CFU/gram to about 10¹¹ CFU/gram, at least about 10⁵ CFU/gram to about 10¹¹ CFU/gram, about 10⁵ CFU/gram to about 10¹⁰ CFU/gram, about 10⁵ CFU/gram to about 10¹² CFU/gram, about 10⁵ CFU/gram to about 10⁶ CFU/gram, about 10⁶ CFU/gram to about 10⁷ CFU/gram, about 10⁷ CFU/gram to about 10⁸ CFU/gram, about 10⁸ CFU/gram to about 10⁹ CFU/gram, about 10⁹ CFU/gram to about 10¹⁰ CFU/gram, about 10¹⁰ CFU/gram to about 10¹¹ CFU/gram, or about 10¹¹ CFU/gram to about 10¹² CFU/gram. In other embodiments, the concentration of the bacterial strain comprises at least about 10⁵ CFU/gram, at least about 10⁶ CFU/gram, at least about 10⁷ CFU/gram, at least about 10⁸ CFU/gram, at least about 10⁹ CFU/gram, at least about 10¹⁰ CFU/gram, at least about 10¹¹ CFU/gram, at least about 10¹² CFU/gram, or at least about 10⁴ CFU/gram. Such concentrations of the bacterial strain can occur in any formulation type of interest, including, for example in a liquid formulation, wettable power, spray dried formulation, cell paste, wettable granule, or freeze-dried formulation.

In some embodiments, the bacterial strain can occur in a liquid formulation. Liquid formulations can comprise an amount of a cell of the bacterial strain AIP61892 or AIP1620 or an active variant thereof, or a spore or a forespore or a combination of cells, forespores and/or spores from AIP61892 or AIP1620 or an active variant thereof, and/or a composition derived therefrom. In liquid formulations, the amount of bacterial strain or an active variant thereof, and/or a composition derived therefrom, disclosed herein can comprise a concentration of at least about 10⁴ to about 10¹¹ CFU/mL, at least about 10⁵ CFU/mL to about 10¹¹ CFU/mL, about 10⁵ CFU/mL to about 10¹⁰ CFU/mL, about 10⁵ CFU/mL to about 10¹² CFU/mL, about 10⁵ CFU/mL to about 10⁶ CFU/mL, about 10⁶ CFU/mL to about 10⁷ CFU/mL, about 10⁷ CFU/mL to about 10⁸ CFU/mL, about 10⁸ CFU/mL to about 10⁹ CFU/mL, about 10⁹ CFU/mL to about 10¹⁰ CFU/mL, about 10¹⁰ CFU/mL to about 10¹¹ CFU/mL, or about 10¹¹ CFU/mL to about 10¹² CFU/mL or at least about 10⁴ CFU/mL, at least about 10⁵ CFU/mL, at least about 10⁶ CFU/mL, at least about 10⁷ CFU/mL, at least about 10⁸ CFU/mL, at least about 10⁹ CFU/mL, at least about 10¹⁰ CFU/mL, at least about 10¹¹ CFU/mL, at least about 10¹² CFU/mL.

Dry formulations such as cell pastes, wettable powders, granules, and spray dried formulations can comprise the bacterial strain AIP61892 or AIP1620 or an active variant thereof, or a spore or a forespore or a combination of cells, forespores or/and spores of any thereof, and/or can comprise a composition derived from AIP61892 or AIP1620 or an active variant thereof. The amount of the bacterial strain in the cell paste or wettable powder can comprise a concentration of the bacterial strain of at least about 10⁵ CFU/gram to about 10¹¹ CFU/gram, about 10⁷ CFU/gram to about 10¹⁰ CFU/gram, about 10⁷ CFU/gram to about 10¹¹ CFU/gram, about 10⁶ CFU/gram to about 10¹⁰ CFU/gram, about 10⁶ CFU/gram to about 10¹¹ CFU/gram, about 10¹¹ CFU/gram to about 10¹² CFU/gram, about 10⁵ CFU/gram to about 10¹⁰ CFU/gram, about 10⁵ CFU/gram to about 10¹² CFU/gram, about 10⁵ CFU/gram to about 10⁶ CFU/gram, about 10⁶ CFU/gram to about 10⁷ CFU/gram, about 10⁷ CFU/gram to about 10⁸ CFU/gram, about 10⁸ CFU/gram to about 10⁹ CFU/gram, about 10⁹ CFU/gram to about 10¹⁰ CFU/gram, about 10¹⁰ CFU/gram to about 10¹¹ CFU/gram, or about 10¹¹ CFU/gram to about 10¹² CFU/gram. In some embodiments, the concentration of the bacterial strain comprises at least about 10⁵ CFU/gram, at least about 10⁶ CFU/gram, at least about 10⁷ CFU/gram, at least about 10⁸ CFU/gram, at least about 10⁹ CFU/gram, at least about 10¹⁰ CFU/gram, at least about 10¹¹ CFU/gram, at least about 10¹² CFU/gram, or at least about 10¹³ CFU/gram.

As used herein, a “cell paste” comprises a population of cells that has been centrifuged and/or filtered and/or otherwise concentrated.

Further provided is a coated seed which comprises a seed and a coating on the seed, wherein the coating comprises at least one bacterial strain such as AIP61892 or an active variant thereof, or a spore or a forespore or a combination of cells, forespores or/and spores, and/or can comprise a composition derived from AIP61892 or an active variant thereof, wherein said bacterial strain or the active variant thereof is present on the seed at about 10⁵ CFU/100 lbs of seed to about 10¹⁰ CFU/100 lbs of seed, at about 10⁶ CFU/100 lbs of seed to about 10¹¹ CFU/100 lbs of seed, at about 10⁸ CFU/100 lbs of seed to about 10⁹ CFU/100 lbs of seed, at about 10⁸ CFU/100 lbs of seed to about 10¹⁰ CFU/100 lbs of seed, at about 10⁶ CFU/100 lbs of seed to about 10¹¹ CFU/100 lbs of seed, or at about 10⁷ CFU/100 lbs of seed to about 10¹⁴ CFU/100 lbs of seed. Various plants of interest are disclosed elsewhere herein.

A seed coating can further comprise at least at least one nutrient, at least one herbicide or at least one pesticide, or at least one biocide. See, for example, US App Pub. 20040336049, 20140173979, and 20150033811.

In other embodiments, the viability of the bacterial strain AIP61892 or AIP1620 or an active variant thereof in a composition or formulation can be quantitated using an epifluorescence assay in which fluorescent dyes that are specific for cells with intact membranes or disrupted membranes are utilized, such as those assays that use a SYTO 9 nucleic acid stain that fluoresces green indicating a cell has an intact membrane and propidium iodide that fluoresces red indicating a cell with a disrupted membrane that is not viable (see, for example, LIVE/DEAD® BacLight™ Bacterial Viability and Counting Kit from Molecular Probes; and Ivanova et al. (2010) Biotechnology & Biotechnological Equipment 24:supl, 567-570). For Bacillus spp., it has been shown in the art that dormant spores do not stain readily with either SYTO9 or propidium iodide, however dead dormant spores will stain red (Ghosh et al. (2017) Scientific Reports 7: 17768). Intact Bacillus spp. spores are also readily detected as phase bright in phase contrast microsopy. All cells with an intact membrane may be counted as “total cells” when quantifying a composition comprising bacterial strain AIP1620 or AIP050999, for example as total cells/gram or total cells/ml.

It is known that following desiccation, some Pseudomonas strains enter a metabolically active state in which the cells are viable but not culturable (VBNC) (Pazos-Rojas et al. (2019) PLoS ONE 14(7):e0219554). Cells in a VBNC state retain the ability to be cultured if reconstituted, for example, in water or root exudates, when exposed to particular metals or ions, or any other reconstitution method that is specific for the individual VBNC bacterial strain.

In some embodiments, the composition or formulation comprises a concentration (e.g., as measured by viability) of the bacterial strain of at least about 10¹ cells/gram to about 10¹⁵ cells/gram, where “cells” includes viable cells, live dormant spores, and germinating spores. In some embodiments, the composition or formulation comprises a concentration of the bacterial strain of 10² cells/gram to about 10⁵ cells/gram, 10² cells/gram to about 10⁴ cells/gram, 10³ cells/gram to about 10⁶ cells/gram, 10⁴ cells/gram to about 10⁸ cells/gram, at least about 10⁵ cells/gram to about 10¹¹ cells/gram, about 10⁷ cells/gram to about 10¹¹ cells/gram, about 10⁷ cells/gram to about 10¹³ cells/gram, about 10⁶ cells/gram to about 10¹¹ cells/gram, about 10⁶ cells/gram to about 10¹³ cells/gram, about 10¹⁰ cells/gram to about 10¹² cells/gram, about 10⁸ cells/gram to about 10¹³ cells/gram, about 10⁹ cells/gram to about 10¹⁴ cells/gram, about 10⁸ cells/gram to about 10¹² cells/gram, about 10⁸ cells/gram to about 10¹² cells/gram, about 10⁹ cells/gram to about 10¹² cells/gram, about 10¹⁰ cells/gram to about 10¹¹ cells/gram, about 10¹¹ cells/gram to about 10¹² cells/gram, about 10¹¹ cells/gram to about 10¹² cells/gram, or about 10¹² cells/gram to about 10¹³ cells/gram, where “cells” includes viable cells, live dormant spores, and germinating spores. In some embodiments, the concentration of the bacterial strain comprises at least about 10² cells/gram, at least about 10³ cells/gram, at least about 10⁴ cells/gram, at least about 10⁵ cells/gram, at least about 10⁶ cells/gram, at least about 10⁷ cells/gram, at least about 10⁸ cells/gram, at least about 10⁹ cells/gram, at least about 10¹⁰ cells/gram, at least about 10¹¹ cells/gram, at least about 10¹² cells/gram, at least about 10¹³ cells/gram, at least about 10¹⁴ cells/gram, or at least about 10¹⁵ cells/gram of viable cells, live dormant spores, or germinating spores as measured with an epifluorescence assay and/or phase contrast microscopy.

In liquid compositions and formulations, the amount of bacterial strain, or an active variant thereof, disclosed herein can comprise a concentration of at least about 10¹ cells/mL to about 10¹⁵ cells/mL, where “cells” includes viable cells, live dormant spores, and germinating spores. In some embodiments, the composition or formulation comprises a concentration of the bacterial strain of 10² cells/mL to about 10⁶ cells/mL, 10⁵ cells/mL to about 10¹⁰ cells/mL, 10⁸ cells/mL to about 10¹⁵ cells/mL, 10⁹ cells/mL to about 10¹² cells/mL, at least about 10³ to about 10⁹ cells/mL, at least about 10³ to about 10⁶ cells/mL, at least about 10⁴ to about 10¹¹ cells/mL, at least about 10⁸ cells/mL to about 10¹³ cells/mL, about 10⁵ cells/mL to about 10¹⁰ cells/mL, about 10⁵ cells/mL to about 10¹² cells/mL, about 10⁸ cells/mL to about 10¹⁵ cells/mL, about 10⁸ cells/mL to about 10¹² cells/mL, about 10⁷ cells/mL to about 10¹¹ cells/mL, about 10⁸ cells/mL to about 10¹¹ cells/mL, about 10⁹ cells/mL to about 10¹⁰ cells/mL, about 10¹⁰ cells/mL to about 10¹¹ cells/mL, or about 10¹¹ cells/mL to about 10¹² cells/mL or at least about 10³ cells/mL, at least about 10⁴ cells/mL, at least about 10⁵ cells/mL, at least about 10⁶ cells/mL, at least about 10⁷ cells/mL, at least about 10⁸ cells/mL, at least about 10⁹ cells/mL, at least about 10¹⁰ cells/mL, at least about 10¹¹ cells/mL, at least about 10¹² cells/mL, at least about 10¹³ cells/mL, at least about 10¹⁴ cells/mL, or at least about 10¹⁵ cells/mL, where “cells” includes viable cells, live dormant spores, and germinating spores, as measured with an epifluorescence assay and/or phase contrast microscopy.

It is known that bacterial strains with pesticidal activity produce a battery of secondary metabolites which can serve as antibacterial and antifungal compounds (Lucke et al. (2020) Frontiers in Plant Science 11: Article 589416). In some embodiments, the concentration of a secondary metabolite within a composition or formulation comprising a bacterial strain, such as AIP61892 or an active variant thereof, can be measured as a surrogate of the viability and/or pesticidal activity of the bacterial strain in the composition or formulation. For example, pyrrolnitrin can be measured as a reporter metabolite for antifungal activity as it is co-regulated with other antifungal metabolites that are active in Pseudomonas spp. The presence of pyrrolnitrin is a measure of intact cells and cell concentration within a composition or formulation. Pyrrolnitrin and other antifungal metabolites are retained within cells and not secreted, so measurement first requires cell lysis. Pyrrolnitrin can then be measured using any analytical chemistry method known in the art, including but not limited to, high performance liquid chromatography with ultraviolet detection (HPLC-UV) of a composition or formulation, such as that described in Hill et al. (1994) Appl Env Micro 60(1) 78-85, which is herein incorporated by reference in its entirety.

Microbes such as Bacillus spp. produce a variety of secondary metabolites with pesticidal properties, such as polyketides, peptide antibiotics, bacteriocins, and cyclic lipopeptides. Polyketides include bacillaene, difficidin, macrolactin, aurantinins, and basiliskamide. Cylic lipopeptides include those in the surfactin, iturin, fengycin, and kurstakin families. The presence and/or concentration of a secondary metabolite such as a cyclic lipopeptide or polyketide many be used as a surrogate of the viability and/or pesticidal activity of the bacterial strain in the composition or formulation. For example, lipopeptides may be extracted from a bacterial culture using methods well known in the art, such as a combination of acid precipitation and solvent extraction. The lipopeptide may then be purified and measured by a variety of methods, including membrane ultrafiltration, ionic exchange chromatography, adsorption-desorption on resins, HPLC-UV, hydrophobic interaction chromatography, and/or gel filtration (Ines and Dhouha (2015) Peptides 71: 100-112).

In some embodiments, the presently disclosed compositions or formulations comprise between about 100 μg/g to 2000 μg/g, 200 μg/g to 1800 μg/g, 300 μg/g to 1500 μg/g, 300 μg/g to 1300 μg/g, 400 μg/g to 1500 μg/g, 400 μg/g to 1300 μg/g, 300 μg/g to 1000 μg/g, 400 μg/g to 1000 μg/g, 500 μg/g to 1000 μg/g, 500 μg/g to 1300 μg/g, 600 μg/g to 1000 μg/g, 600 μg/g to 1300 μg/g, 600 μg/g to 1500 μg/g, or about 300 μg/g, about 400 μg/g, about 500 μg/g, about 600 μg/g, about 700 μg/g, about 800 μg/g, about 900 μg/g, about 1000 μg/g, about 1100 μg/g, about 1200 μg/g, about 1300 μg/g, about 1400 μg/g, about 1500 μg/g, about 1500 μg/g, about 1600 μg/g, about 1700 μg/g, about 1800 μg/g, about 1900 μg/g, and about 2000 μg/g expressed as μg of of a secondary metabolite per g of bacteria. In further embodiments, the secondary metabolite may be pyrrolnitrin, bacillaene, difficidin, macrolactin, aurantinins, basiliskamide, or a member of the tensin, pseudophomin, massetolid, pseudodesmin, xantholysin, syringomycin, surfactin, iturin, fengycin, or kurstakin families.

Further provided is a composition comprising a whole cell broth, supernatant, filtrate, or extract derived from bacterial strain AIP61892 or AIP1620 or an active variant thereof, wherein an effective amount of the composition improves an agronomic trait of interest of a plant or controls a plant pest or a plant pathogen that causes disease. The composition contains effective compound(s), metabolite(s), and/or protein(s) which improve an agronomic trait of interest of a plant or controls a plant pest or a plant pathogen that causes disease. The supernatant refers to the liquid remaining when cells are grown in broth or are harvested in another liquid from an agar plate and are removed by centrifugation, filtration, sedimentation, or other means well known in the art. The supernatant may be further concentrated to produce a filtrate. The filtrate may comprise a concentrated amount of an effective compound or metabolite compared to the concentration of the effective compound or metabolite in the supernatant or whole cell broth. In some embodiments, the supernatant, filtrate, or extract may be processed to a wettable powder, spray dried formulation, and/or seed coating. In other embodiments, the supernatant, filtrate, or extract may be concentrated (e.g., water is removed) but remain in a liquid formulation. The composition described above can be applied alone or in combination with another substance, in an effective amount to control a plant pest or improve an agronomic trait of interest of a plant. The various formulations disclosed herein can be stable for at least 30, 40, 50, 60, 70, 80, 90, 100, 125, 150, 200, 225, 250, 275, 300, 325, 350 days, 1.5 years, 2 years or longer. By “stable formulation” it is intended that the formulation retains viable bacteria and/or retains an effective amount of a biologically active bacterial population. The bacterial population may comprise bacterial cells, spores, forespores, or a combination of any of these. Biological activity as used herein refers to the ability of the formulation to improve an agronomic trait of interest or control a plant pest or a plant pathogen that causes a plant disease. In one embodiment, the stable formulation retains at least about 1%, about 10%, about 20%, about 30% about 40%, about 50%, about 60%, about 70%, about 80%, or about 90% of CFU/gram or cells/g in the formulation at a given storage time point when compared to the CFU/gram or cells/g produced after immediate preparation of the formulation. In another embodiment, the stable formulation retains at least about 30% to 80%, about 50% to about 80%, about 60% to about 70%, about 70% to about 80%, about 40% to about 50%, about 50% to about 60%, about 60% to about 70% of biological activity (e.g., antifungal activity as calculated using a reporter metabolite such as pyrrolnitrin) in the formulation at a given storage time point when compared to the biological activity found in the formulation immediately after production. In another embodiment, the stable formulation at a given storage time point retains at least about 30%, 45%, 50%, 60%, 70%, 80%, 90% of biological activity when compared to the biological activity found in the formulation immediately after production. In still another embodiment, the stable formation retains any combination of the viability and biological activity noted above.

The formulations preferably comprise between 0.00000001% and 98% by weight of active compound or, with particular preference, between 0.01% and 95% by weight of active compound, more preferably between 0.5% and 90% by weight of active compound, based on the weight of the formulation.

The active compound content of the application forms prepared from the formulations may vary within wide ranges. The active compound concentration of the application forms may be situated typically between 0.00000001% and 95% by weight of active compound, preferably between 0.00001% and 1% by weight, based on the weight of the application form. Application takes place in a customary manner adapted to the application forms.

In some embodiments, the composition or formulation comprises AIP61892 or an active variant thereof and a copper compound or AIP1620 or an active variant thereof at various active ingredient weight ratios. The phrase “active ingredient weight ratio” refers to the quantitative relation between the weights of each of two active ingredients within a composition. The active ingredients of the presently disclosed compositions are those that have the ability to control a plant pest or pathogen or improve at least one agronomic trait when applied in an effective amount to a plant, plant part, or an area of cultivation, including for example, reducing plant disease severity and/or reducing plant disease development. Specifically, the active ingredients of the presently disclosed compositions are a bacterial strain, such as AIP61892 or AIP1620, or active variant of any thereof, or a copper compound. When calculating the active ingredient weight ratio of a composition comprising a bacterial strain, such as AIP61892 and AIP1620, or active variant of any thereof, the total weight of the bacterial strain, regardless of viability or culturability, can be used to calculate the active ingredient weight ratio.

The active ingredient weight ratio can be measured using any method known in the art, including weighing the amount of a dry formulation comprising one or both of the active ingredients to obtain the formulation weight and then calculating the weight of each active ingredient based on the reported, known, or calculated percentage of the active ingredient to the total formulation weight (w/w). For example, 2 pounds of a dry formulation that is 50% w/w of the active ingredient copper compound comprises 1 pound of the copper compound. Alternatively, if the composition is a liquid composition, the weight of each active ingredient can be calculated by measuring the total volume of the formulation and then calculating the weight of each active ingredient based on the reported, known, or calculated percentage of the active ingredient to the total formulation volume (w/v). Percent weight per volume is defined as the grams of solute in 100 ml of a solution. For example, 100 ml of a liquid composition that is 50% w/v of the active ingredient copper compound comprises 50 g of the copper compound. One non-limiting method that can be used to measure the weight of a bacterial strain (cells, spores, etc.) or an active variant thereof in liquid culture includes pelleting the cells using, for example, a centrifuge, in order to remove any liquid and then weighing the pelleted cells. The effectiveness of a given bacterial cell weight can be expressed by measuring the culturability, viability or activity (e.g., measuring a reporter metabolite such as pyrrolnitrin), as discussed elsewhere herein. In certain embodiments, the culturablity of a bacterial strain, such as AIP61892 or AIP1620, or active variant of any thereof, in a composition or formulation can be quantitated by measuring the number of colony forming units per gram or per ml of the formulation. In some embodiments, the composition or formulation comprises a concentration of the bacterial strain of at least about 10⁴ to about 10¹² CFU/gram, 10⁴ to about 10¹⁰ CFU/gram at least about 10⁵ CFU/gram to about 10¹¹ CFU/gram, about 10⁵ CFU/gram to about 10¹⁰ CFU/gram, about 10⁵ CFU/gram to about 10¹² CFU/gram, about 10⁵ CFU/gram to about 10⁶ CFU/gram, about 10⁶ CFU/gram to about 10⁷ CFU/gram, about 10⁷ CFU/gram to about 10⁸ CFU/gram, about 10⁸ CFU/gram to about 10⁹ CFU/gram, about 10⁹ CFU/gram to about 10¹⁰ CFU/gram, about 10¹⁰ CFU/gram to about 10¹¹ CFU/gram, or about 10¹¹ CFU/gram to about 10¹² CFU/gram. In other embodiments, the concentration of the bacterial strain comprises at least about 10⁴ CFU/gram, at least about 10⁵ CFU/gram, at least about 10⁶ CFU/gram, at least about 10⁷ CFU/gram, at least about 10⁸ CFU/gram, at least about 10⁹ CFU/gram, at least about 10¹⁰ CFU/gram, at least about 10¹¹ CFU/gram, or at least about 10¹² CFU/gram, or equivalent measure of bacterial concentration. In some embodiments, the composition or formulation comprises a concentration of the bacterial strain of at least about 10⁴ to about 10¹² CFU/mL, 10⁴ to about 10¹⁰ CFU/mL at least about 10⁵ CFU/mL to about 10¹¹ CFU/mL, about 10⁵ CFU/mL to about 10¹⁰ CFU/mL, about 10⁵ CFU/mL to about 10¹² CFU/mL, about 10⁵ CFU/mL to about 10⁶ CFU/mL, about 10⁶ CFU/mL to about 10⁷ CFU/mL, about 10⁷ CFU/mL to about 10⁸ CFU/mL, about 10⁸ CFU/mL to about 10⁹ CFU/mL, about 10⁹ CFU/mL to about 10¹⁰ CFU/mL, about 10¹⁰ CFU/mL to about 10¹¹ CFU/mL, or about 10¹¹ CFU/mL to about 10¹² CFU/mL. In other embodiments, the concentration of the bacterial strain comprises at least about 10⁴ CFU/mL, at least about 10⁵ CFU/mL, at least about 10⁶ CFU/mL, at least about 10⁷ CFU/mL, at least about 10⁸ CFU/mL, at least about 10⁹ CFU/mL, at least about 10¹⁰ CFU/mL, at least about 10¹¹ CFU/mL, or at least about 10¹² CFU/mL, or equivalent measure of bacterial concentration.

In some embodiments, AIP61892 or a variant thereof and a copper compound can be combined (in a formulation or applied in combination, simultaneously or sequentially) in an active ingredient weight ratio of about 1:1000 (bacterial strain:copper compound) to about 1000:1, including but not limited to about 1:1000, 1:500, 1:100, 1:10, 1:5, 1:2, 1:1, 2:1, 5:1, 10:1, 100:1, 500:1, and 1000:1.

In some embodiments, AIP61892 or a variant thereof and AIP1620 or a variant thereof can be combined (in a formulation or applied in combination, simultaneously or sequentially) in an active ingredient weight ratio of about 1:1000 (AIP61892:AIP1620) to about 1000:1, including but not limited to about 1:1000, 1:500, 1:100, 1:10, 1:5, 1:2, 1:1, 2:1, 5:1, 10:1, 100:1, 500:1, and 1000:1.

In some cases, the combination (simultaneous or sequential application to a plant or an area of cultivation) of AIP61892 or an active variant thereof and copper compound or AIP1620 or an active variant thereof may exhibit an additive effect on controlling a plant pathogen or treating or preventing a plant disease or improving an agronomic trait of interest. In other embodiments, the combination of AIP61892 or an active variant thereof and copper compound or AIP1620 or an active variant thereof may show synergistic activity where the mixture of the two exceeds that expected from their simple additive effect. In particular embodiments, the simultaneous or sequential application of AIP61892 or an active variant thereof and copper compound or AIP1620 or an active variant thereof to a plant or an area of cultivation results in the controlling of a plant pathogen or treatment or prevention of a plant disease or an improvement of an agronomic trait of interest wherein no such effect results when either of the bacterial strains or copper compound are used alone.

In specific embodiments, the compositions and formulations disclosed herein include AIP61892 or an active variant thereof and a copper compound or AIP1620 or an active variant thereof that comprise less than the suggested amount of either of the bacterial strains or the copper compound. The term “suggested amount,” “standard amount,” “suggested rate,” or “standard rate” in reference to a chemical fungicide or bacteria refers to an amount or rate that is the amount or rate that effectively controls a plant pathogen, treats or prevents a plant disease, or improves an agronomic trait of interest in a plant when used alone (i.e., not in conjunction with an additional fungicide). The suggested or standard amount or rate can be the amount or rate approved for use by an applicable government agency or the amount or rate suggested by the manufacturer or listed on the label of a commercial product comprising the chemical fungicide or bacteria. The suggested amount can differ based on the particular plant pathogen being targeted or plant disease being treated or prevented or the particular agronomic trait of interest that is desired to be improved or the particular plant that the bacteria and chemical fungicide is being applied thereto, the particular type of application (e.g., foliar, field inoculation), or the like. For example, the compositions and formulations can comprise a bacterial strain for application at about 20%, 25%, 30%, 35%, 40%, 45%, 50%, 55%, 60%, 65%, 70%, 75%, 80%, 85%, 90%, 95%, or about 20-70%, about 30-60%, about 30-70%, about 40-80%, about 40-70%, about 40-60%, about 40-50%, about 50-90%, about 50-80%, about 50-70%, about 50-60%, about 60-90%, about 60-80%, about 60-70%, about 70-90%, about 70-90%, or about 80-90% of the suggested amount for application when used alone. The suggested amount for application of the bacterial strain can be about 1 lb/acre, about 1.25 lb/acre, about 1.5 lb/acre, about 1.75 lb/acre, about 2 lb/acre, about 2.25 lb/acre, about 2.5 lb/acre, about 2.75 lb/acre, about 3 lb/acre, about 3.5 lb/acre, about 4 lb/acre, about 5 lb/acre, about 6 lb/acre, about 7 lb/acre, about 8 lb/acre, about 1-10 lb/acre, or about 1-8 lb/acre. A formulated product comprising AIP61892 or AIP1620 may have 50% AIP61892 or AIP1620 by weight or be provided in any weight ratio relative to the other bacterial strain or copper compound. The application rate of the copper compound can be calculated based on the rate of the bacterial strain, or active variant thereof, according to the selected active ingredient weight ratio as disclosed elsewhere herein. Application of 5 lbs/acre of a 50% formulated product, for example, comprises 2.5 lbs/acre of the active ingredient.

The compositions and formulation comprising a bacterial strain can comprise a copper compound at about 20%, 25%, 30%, 35%, 40%, 45%, 50%, 55%, 60%, 65%, 70%, 75%, 80%, 85%, 90%, 95%, or about 20-70%, about 30-60%, about 30-70%, about 40-80%, about 40-70%, about 40-60%, about 40-50%, about 50-90%, about 50-80%, about 50-70%, about 50-60%, about 60-90%, about 60-80%, about 60-70%, about 70-90%, about 70-90%, or about 80-90% of the suggested amount for application when used alone.

The bacterial strain AIP61892 provided herein or an active variant thereof can be mixed with an additional biocide, such as a fungicide, bactericide, nematicide, insecticide, or herbicide to enhance its activity or the activity of the chemical to which it has been added.

In specific embodiments, the bacterial strain AIP61892 or an active variant thereof and/or a composition derived therefrom is compatible with agricultural chemicals used to improve performance of biocides. Such agricultural chemicals include safeners, surfactants, stickers, spreaders, UV protectants, and suspension and dispersal aids. Safeners are chemicals that improve or modify the performance of herbicides. Surfactants, spreaders, and stickers are chemicals included in agricultural spray preparations that change the mechanical properties of the spray (for example, by altering surface tension or improving leaf cuticle penetration). UV protectants improve the performance of agricultural biocides by reducing degradation by ultraviolet light. Suspension and dispersal aids improve the performance of biocides by altering their behavior in a spray tank. In instances where the bacterial strain is not compatible with an agricultural chemical of interest, if desired, methods can be undertaken to modify the bacterial strain to impart the compatibility of interest. Such methods to produce modified bacterial strains include both selection techniques and/or transformation techniques.

The AIP61892 bacterial strain provided herein, an active variant thereof, and/or a composition derived therefrom can be used to significantly improve at least one agronomic trait of interest (i.e, reduce disease caused by a plant pathogen (e.g., fungal pathogen or fungal-like pathogen) or reduce susceptibility to plant pests). The bacterial strain provided herein, an active variant thereof, and/or a composition derived therefrom can be used with other pesticides for an effective integrated pest management program. In one embodiment, the biocontrol populations can be mixed with known pesticides in a manner described in WO 94/10845, herein incorporated by reference.

Non-limiting examples of compounds and compositions that can be added to the formulation, include but are not limited to, Acetyl tributyl citrate [Citric acid, 2-(acetyloxy)-, tributyl ester]; Agar; Almond hulls; Almond shells; alpha-Cyclodextrin; Aluminatesilicate; Aluminum magnesium silicate [Silicic acid, aluminum magnesium salt]; Aluminum potassium sodium silicate [Silicic acid, aluminum potassium sodium salt]; Aluminum silicate; Aluminum sodium silicate [Silicic acid, aluminum sodium salt]; Aluminum sodium silicate (1:1:1)[Silicic acid (H4SiO4), aluminum sodium salt (1:1:1)]; Ammonium benzoate [Benzoic acid, ammonium salt]; Ammonium stearate [Octadecanoic acid, ammonium salt]; Amylopectin, acid-hydrolyzed, 1-octenylbutanedioate; Amylopectin, hydrogen 1-octadecenylbutanedioate; Animal glue; Ascorbyl palmitate; Attapulgite-type clay; Beeswax; Bentonite; Bentonite, sodian; beta-Cyclodextrin; Bone meal; Bran; Bread crumbs; (+)-Butyl lactate; [Lactic acid, n-butyl ester, (S)]; Butyl lactate [Lactic acid, n-butyl ester]; Butyl stearate [Octadecanoic acid, butyl ester]; Calcareous shale; Calcite (Ca(Co₃)); Calcium acetate; Calcium acetate monohydrate [Acetic acid, calcium salt, monohydrate]; Calcium benzoate [Benzoic acid, calcium salt]; Calcium carbonate; Calcium citrate [Citric acid, calcium salt]; Calcium octanoate; Calcium oxide silicate (Ca₃O(SiO₄)); Calcium silicate [Silicic acid, calcium salt]; Calcium stearate [Octadecanoic acid, calcium salt]; Calcium sulfate; Calcium sulfate dehydrate; Calcium sulfate hemihydrate; Canary seed; Carbon; Carbon dioxide; Carboxymethyl cellulose [Cellulose, carboxymethyl ether]; Cardboard; Carnauba wax; Carob gum [Locust bean gum]; Carrageenan; Caseins; Castor oil; Castor oil, hydrogenated; Cat food; Cellulose; Cellulose acetate; Cellulose, mixture with cellulose carboxymethyl ether, sodium salt; Cellulose, pulp; Cellulose, regenerated; Cheese; Chlorophyll a; Chlorophyll b; Citrus meal; Citric acid; Citric acid, monohydrate; Citrus pectin; Citrus pulp; Clam shells; Cocoa; Cocoa shell flour; Cocoa shells; Cod-liver oil; Coffee grounds; Cookies; Cork; Corn cobs; Cotton; Cottonseed meal; Cracked wheat; Decanoic acid, monoester with 1,2,3-propanetriol; Dextrins; Diglyceryl monooleate [9-Octadecenoic acid, ester with 1,2,3-propanetriol]; Diglyceryl monostearate [9-Octadecanoic acid, monoester with xybis(propanediol)]; Dilaurin [Dodecanoic acid, diester with 1,2,3-propanetriol]; Dipalmitin [Hexadecanoic acid, diester with 1,2,3-propanetriol]; Dipotassium citrate [Citric acid, dipotassium salt]; Disodium citrate [Citric acid, disodium salt]; Disodium sulfate decahydrate; Diatomaceous earth (less than 1% crystalline silica); Dodecanoic acid, monoester with 1,2,3-propanetriol; Dolomite; Douglas fir bark; Egg shells; Eggs; (+)-Ethyl lactate [Lactic acid, ethyl ester, (S)]; Ethyl lactate [Lactic acid, ethyl ester]; Feldspar; Fish meal; Fish oil (not conforming to 40 CFR 180.950); Fuller's earth; Fumaric acid; gamma-Cyclodextrin; Gelatins; Gellan gum; Glue (as depolymd. animal collagen); Glycerin [1,2,3-Propanetriol]; Glycerol monooleate [9-Octadecenoic acid (Z)-, 2,3-dihydroxypropyl ester]; Glyceryl dicaprylate [Octanoic acid, diester with 1,2,3-propanetriol]; Glyceryl dimyristate [Tetradecanoic acid, diester with 1,2,3-propanetriol]; Glyceryl dioleate [9-Octadecenoic acid (9Z)-, diester with 1,2,3-propanetriol]; Glyceryl distearate; Glyceryl monomyristate [Tetradecanoic acid, monoester with 1,2,3-propanetriol]; Glyceryl monooctanoate [Octanoic acid, monoester with 1,2,3-propanetriol]; Glyceryl monooleate [9-Octadecenoic acid (9Z)-, monoester with 1,2,3-propanetriol]; Glyceryl monostearate [Octadecanoic acid, monoester with 1,2,3-propanetriol]; Glyceryl stearate [Octadecanoic acid, ester with 1,2,3-propanetriol]; Granite; Graphite; Guar gum; Gum Arabic; Gum tragacanth; Gypsum; Hematite (Fe₂O₃); Humic acid; Hydrogenated cottonseed oil; Hydrogenated rapeseed oil; Hydrogenated soybean oil; Hydroxyethyl cellulose [Cellulose, 2-hydroxyethyl ether]; Hydroxypropyl cellulose [Cellulose, 2-hydroxypropyl ether]; Hydroxypropyl methyl cellulose [Cellulose, 2-hydroxypropyl methyl ether]; Iron magnesium oxide (Fe₂MgO₄); Iron oxide (Fe₂O₃); Iron oxide (Fe₂O₃); Iron oxide (Fe₃O₄); Iron oxide (FeO); Isopropyl alcohol [2-Propanol]; Isopropyl myristate; Kaolin; Lactose; Lactose monohydrate; Lanolin; Latex rubber; Lauric acid; Lecithins; Licorice extract; Lime (chemical) dolomitic; Limestone; Linseed oil; Magnesium carbonate [Carbonic acid, magnesium salt (1:1); Magnesium benzoate; Magnesium oxide; Magnesium oxide silicate (Mg₃O(Si₂O₅)₂), monohydrate; Magnesium silicate; Magnesium silicate hydrate; Magnesium silicon oxide (Mg₂Si₃O₈); Magnesium stearate [Octadecanoic acid, magnesium salt]; Magnesium sulfate; Magnesium sulfate heptahydrate; Malic acid; Malt extract; Malt flavor; Maltodextrin; Methylcellulose [Cellulose, methyl ether]; Mica; Mica-group minerals; Milk; N/A Millet seed; Mineral oil (U.S.P.); 1-Monolaurin [Dodecanoic acid, 2,3-dihydroxypropyl ester]; 1-Monomyristin [Tetradecanoic acid, 2,3-dihydroxypropyl ester]; Monomyristin [Decanoic acid, diester with 1,2,3-propanetriol]; Monopalmitin [Hexadecanoic acid, monoester with 1,2,3-propanetriol]; Monopotassium citrate [Citric acid, monopotassium salt; Monosodium citrate [Citric acid, monosodium salt]; Montmorillonite; Myristic acid; Nepheline syenite; Nitrogen; Nutria meat; Nylon; Octanoic acid, potassium salt; Octanoic acid, sodium salt; Oils, almond; Oils, wheat; Oleic acid; Oyster shells; Palm oil; Palm oil, hydrogenated; Palmitic acid [Hexadecanoic acid]; Paraffin wax; Peanut butter; Peanut shells; Peanuts; Peat moss; Pectin; Perlite; Perlite, expanded; Plaster of paris; Polyethylene; Polyglyceryl oleate; Polyglyceryl stearate; Potassium acetate [Acetic acid, potassium salt]; Potassium aluminum silicate, anhydrous; Potassium benzoate [Benzoic acid, potassium salt]; Potassium bicarbonate [Carbonic acid, monopotassium salt]; Potassium chloride; Potassium citrate [Citric acid, potassium salt]; Potassium humate [Humic acids, potassium salts]; Potassium myristate [Tetradecanoic acid, potassium salt]; Potassium oleate [9-Octadecenoic acid (9Z)-, potassium salt; Potassium ricinoleate [9-Octadecenoic acid, 12-hydroxy-, monopotassium salt, (9Z,12R)-]; Potassium sorbate [Sorbic acid, potassium salt]; Potassium stearate [Octadecanoic acid, potassium salt]; Potassium sulfate; Potassium sulfate [Sulfuric acid, monopotassium salt]; 1,2-Propylene carbonate [1,3-Dioxolan-2-one, 4-methyl-]; Pumice; Red cabbage color (expressed from edible red cabbage heads via a pressing process using only acidified water); Red cedar chips; Red dog flour; Rubber; Sawdust; Shale; Silica, amorphous, fumed (crystalline free); Silica, amorphous, precipated and gel; Silica (crystalline free); Silica gel; Silica gel, precipitated, crystalline-free; Silica, hydrate; Silica, vitreous; Silicic acid (H₂SiO₃), magnesium salt (1:1); Soap (The water soluble sodium or potassium salts of fatty acids produced by either the saponification of fats and oils, or the neutralization of fatty acid); Soapbark [Quillaja saponin]; Soapstone; Sodium acetate [Acetic acid, sodium salt]; Sodium alginate; Sodium benzoate [Benzoic acid, sodium salt]; Sodium bicarbonate; Sodium carboxymethyl cellulose [Cellulose, carboxymethyl ether, sodium salt]; Sodium chloride; Sodium citrate; Sodium humate [Humic acids, sodium salts]; Sodium oleate; Sodium ricinoleate [9-Octadecenoic acid, 12-hydroxy-, monosodium salt, (9Z,12R)-]; Sodium stearate [Octadecanoic acid, sodium salt]; Sodium sulfate; Sorbitol [D-glucitol]; Soy protein; Soya lecithins [Lecithins, soya]; Soybean hulls; Soybean meal; Soybean, flour; Stearic acid [Octadecanoic acid]; Sulfur; Syrups, hydrolyzed starch, hydrogenated; Tetragylceryl monooleate [9-Octadecenoic acid (9Z)-, monoester with tetraglycerol]; Tricalcium citrate [Citric acid, calcium salt (2:3)]; Triethyl citrate [Citric acid, triethyl ester; Tripotassium citrate [Citric acid, tripotassium salt]; Tripotassium citrate monohydrate [Citric acid, tripotassium salt, monohydrate]; Trisodium citrate [Citric acid, trisodium salt]; Trisodium citrate dehydrate [Citric acid, trisodium salt, dehydrate]; Trisodium citrate pentahydrate [Citric acid, trisodium salt, pentahydrate]; Ultramarine blue [C.I. Pigment Blue 29]; Urea; Vanillia; Vermiculite; Vinegar (maximum 8% acetic acid in solution); Vitamin C [L-Ascorbic acid]; Vitamin; Walnut flour; Walnut shells; Wheat; Wheat flour; Wheat germ oil; Whey; White mineral oil (petroleum); Wintergreen oil; Wollastonite (Ca(SiO3)); Wool; Xanthan gum; Yeast; Zeolites (excluding erionite (CAS Reg. No. 66733-21-9)); Zeolites, NaA; Zinc iron oxide; Zinc oxide (ZnO); and Zinc stearate [Octadecanoic acid, zinc salt].

### IV. Methods of Use

The bacterial strain AIP61892, modified bacterial strains active variants thereof, and/or compositions derived therefrom provided herein can be employed with any plant species to improve an agronomic trait of interest. Agonomic traits of interest include any trait that improves plant health or commercial value. Non-limiting examples of agronomic traits of interest including increase in biomass, increase in drought tolerance, thermal tolerance, herbicide tolerance, drought resistance, nematode resistance, insect resistance, fungus resistance, virus resistance, bacteria resistance, male sterility, cold tolerance, salt tolerance, increased yield, enhanced nutrient use efficiency, increased nitrogen use efficiency, increased tolerance to nitrogen stress, increased fermentable carbohydrate content, reduced lignin content, increased antioxidant content, enhanced water use efficiency, increased vigor, increased germination efficiency, earlier or increased flowering, increased biomass, altered root-to-shoot biomass ratio, enhanced soil water retention, or a combination thereof. In other instance, the agronomic trait of interest includes an altered oil content, altered protein content, altered seed carbohydrate composition, altered seed oil composition, altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, or a detectable modulation in the proteome relative to a reference plant.

In one non-limiting embodiment, the bacterial strain AIP61892, an active variant thereof, and/or a composition derived therefrom provided herein can be employed with any plant species susceptible to a plant disease. By “a plant susceptible to a plant disease” is meant that the causative pathogen(s) of the plant disease are able to infect the plant. For example, a plant susceptible to a plant disease can be susceptible to a plant disease caused by a fungi or fungal-like organism (e.g., an Oomycete such as Phytophthora or Pythium) as disclosed elsewhere herein.

As used herein, the term plant includes plant cells, plant protoplasts, plant cell tissue cultures from which plants can be regenerated, plant calli, plant clumps, and plant cells that are intact in plants or plant parts such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species.

Plants of interest include monocotyledonous plants, also referred to as monocots, and dicotyledonous plants, also referred to as dicots. Examples of plant species of interest include, but are not limited to, species, cultivars, varieties, and hybrids of com (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), grape (Vitus spp.), strawberry (Fragaria x ananassa), cherry (Prunus spp.), apple (Malus domestica), orange (Citrus x sinensis) cashew (Anacardium occidentale), macadamia (Macadamia integrifolia), almond (Prunus amygdalus), sugar beets (Beta vulgaris), sugarcane (Saccharum spp.), Cannabis (including Cannabis sativa, Cannabis indica, and Cannabis ruderalis), oats, barley, vegetables, ornamentals, and conifers.

Plants of interest also include root and tuber vegetables including species, cultivars, varieties, and hybrids of carrot, potato, radish, sweet potato, yam, turnip, rutabaga, arracacha, arrowroot, Chinese artichoke, Jerusalem artichoke, garden beet, burdock, canna, cassava, celeriac, chervil, chicory, chufa, taro/dasheen, ginger, ginseng, horseradish, leren, parsnip, rutabaga, turnip, tanier and sugarbeet; bulb vegetables including species, cultivars, varieties, and hybrids of onion, green onion, leek, and shallot; leafy vegetables such as head lettuce, leaf lettuce, spinach, celery, Swiss chard, watercress, rhubarb, kale, bok choy, endive, collards, cilantro, dandelion, and mustard greens; Brassica head and stem vegetables including species, cultivars, varieties, and hybrids of broccoli, broccoli raab (rapini), gai lon, gai choy, cabbage, Chinese cabbage, Brussels sprouts, cauliflower, kohlrabi, mizuna, mustard greens, and rape greens; legume vegetables including species, cultivars, varieties, and hybrids of bean or pea, including Lupinus spp. including grain lupin, sweet lupin, white lupin, Phaeolus spp. (including green bean, kidney bean, lima bean, navy bean, pinto bean, runner bean, snap bean, tepary bean, wax bean), Vigna spp. (including adzuki bean, asparagus bean, blackeyed pea, catjang, Chinese longbean, cowpea, crowder pea, moth bean, mung bean, rice bean, southern pea, urd bean, and yardlong bean), Pisum spp. (including dwarf pea, edible-podded pea, English pea, field pea, garden pea, green pea, snowpea, and sugar snap pea), fava bean, chickpea, guar, jackbean, lentil; pigeon pea, soybean, sword bean, soybean, succulent cultivars of edible-podded bean or edible-podded pea, succulent shelled cultivars of bean or pea, and dried cultivars of bean or pea; fruiting vegetables including species, cultivars, varieties, and hybrids of tomato, bell peper, non-bell pepper, pimento pepper, chili pepper, eggplant, bush tomato, currant tomato, garden huckleberry, goji berry, ground cherry, okra, tomatillo, sunberry, pepino, African eggplant, scarlet eggplant, pea eggplant, martynia, and roselle; cucurbit vegetables including species, cultivars, varieties, and hybrids of cucumber, muskmelon including hybrids and/or varieties of Cucumis spp. such as cantaloupe, watermelon, and honeydew, squash including pumpkin and winter squash including butternut, calabaza, and acorn squash, and summer squash including Cucurbitaceae family members such as hybrids and/or varieties of Cucurbita pepo such as crookneck squash and straightneck squash, hybrids and/or varieties of Lagenaria spp. such as spaghetti squash, hyotan, and cucuzza, Luffa spp. such as hechima and Chinese okra, Momordica spp. such as bitter melon, balsam pear, and Chinese cucumber, and Sechium edule (chayote); Citrus including species, cultivars, varieties, and hybrids of orange, tangerine, mandarin, lemon, lime, grapefruit, pummelo, tangor, uniq fruit, and kumquat; pome fruit including species, cultivars, varieties, and hybrids of apple, pear, quince, Chinese quince, tejocote, mayhaw, loquat, and azarole; stone fruit including species, cultivars, varieties, and hybrids of sweet cherry, tart cherry, peach, plum, prune plum, apricot, Jujube, nectarine, and plumcot; berry and small fruit including species, cultivars, varieties, and hybrids of blackberry, raspberry, highbush blueberry, caneberry, bushberry, large shrub or tree berry including elderberry, and mulberry, small fruit climbing vine including grape, gooseberry, Amur river grape, may pop, fuzzy kiwifruit, and hardy kiwifruit, low growing berry including strawberry, bearberry, lingonberry, cranberry, cloudberry, muntries, and partridgeberry; tree nut including species, cultivars, varieties, and hybrids of almond, pecan, Brazil nut, butternut, cashew, chestnut, ginkgo, Okari nut, pine nut, pistachio, walnut, and bunya; cereal grain including species, cultivars, varieties, and hybrids of corn (sweet corn and field corn), rice, sorghum, amaranth, lupine, wheat, millet, barely, buckwheat, oats, rye, and wild rice; grass forage, fodder and hay including species, cultivars, varieties, and hybrids of Bermuda grass, bluegrass, bentgrass, bromegrass, sudan grass, switch grass, Poa annua, and fescue; nongrass animal feeds including species, cultivars, varieties, and hybrids of alfalfa, Trifolium spp. and Melilotus spp.; oil seed including species, cultivars, varieties, and hybrids of rapeseed, sunflower seed, cottonseed, canola, calendula, castor oil plant, safflower, poppyseed, sesame, flax, milkweed, tea oil plant, Brassica napus, Brassica campestris, and Crambe abyssinica; stalk, stem, and leaf petiole vegetable including species, cultivars, varieties, and hybrids of asparagus, agave, artichoke, aloe vera, bamboo, fennel, fuki, palm hearts, prickly pear, udo, Chinese celery, and celery; tropical and sub-tropical fruit with an edible peel including species, cultivars, varieties, and hybrids of date, fig, guava, olive; tropical and sub-tropical fruit with an inedible peel including species, cultivars, varieties, and hybrids of atemoya, sugar apple, avocado, mango, papaya, banana, plantains, pomegranate, dragon fruit, lychee, passionfruit, pineapple, durian, and prickly pear; herb including species, cultivars, varieties, and hybrids of basil, mint, marigold, geranium, echinacea, Rooibos, sage, savory, thyme, tarragon, violet, yerba santa, yomogi, and wild bergamot; spice including species, cultivars, varieties, and hybrids of dill seed, celery seed, allspice, anise pepper, anise seed, star anise, annatto, balsam, caraway, cardamom, Cassia, nutmeg, cinnamon, clove, coriander, cumin, echinacea, eucalyptus, fennel, fenugreek, mustard, pepper, pepperbush, peppercorn, peppertree, quinine, rue, saffron, sandalwood, sassafras, sumac, tamarind, vanilla, willow, witch hazel, and yohimbe. Plants of interest also include species, cultivars, varieties, and hybrids of cotton, linen, tobacco, and hops.

Plant of interest include ornamental plants, flowers, flowering plants, tropical foliage, foliage, trees, shrubs, forestry, and grasses, including lawn, sod, turf, and ornamental turf. Plants of interest include annual and perennial flower plants including species, cultivars, varieties, and hybrids of Alyssum, Chrysantheum, Easter lily, Hydrangea, Marigols, Ranunculus, Verbena spp., roses (Rosa spp.), tulips (Tulipa spp.), daffodils (Narcissus spp.), Carnation, Asters, Garden Phlox, Impatiens, Orchids, Roses, Vinca, Azalea, Cyclamen, Geraniums, Kalanchoe, Pansies, Salvia spp., Violas, Begonia, Dianthus, Gerbera, Linaria, Petunia, Snapdragons, Zinnias, Calla lily, Dwarf Bee-balm, Golden Star, Lisianthus, Poinsettia, Stock, Lobelia, Portulaca; tropical foliage including species, cultivars, varieties, and hybrids of Aglaonema, Hibiscus, Dieffenbachia, Leatherleaf Fern, Dracaena spp., English Ivy, and Spathiphyllum; trees and shrubs including species, cultivars, varieties, and hybrids of Azalea boxwood, Gumpo azalea, Ligustrum japonicum, Photinia, Spirea, Crape myrtle, Indian hawthorn, Lilac, Rhododendron, Dogwood, Japanese maple, Loropetalum, Soft Touch holly, Rhododendron spp., Rosaceae spp., Pinus spp. (including loblolly pine, slash pine, ponderosa pine, lodgepole pine, and Monterey pine), Douglas-fir, Western hemlock, Sitka spruce, redwood, true firs such as silver fir and balsam fir, and cedars such as Western red cedar and Alaska yellow-cedar; lawn and turf including species, cultivars, varieties, and hybrids of bluegrass, bentgrass, Bermudagrass, Dichondra, Fescue, Orchardgrass, Poa annua, St. Augustine, Ryegrass, Zoysia, and mixtures thereof.

In specific embodiments, plants of the present invention are row crop plants (for example, corn, alfalfa, sunflower, Brassica spp, Phaaeolus spp., Pisum spp., soybean, cotton, flax, buckwheat, sugarbeets, safflower, peanut, sorghum, sugarcane, wheat, millet, tobacco, etc.). In other embodiments, corn and soybean plants are preferred, and in yet other embodiments corn plants are preferred. In other embodiments, plants of the present invention are permanent crops (for example, wine grapes, pistachios, walnuts, almonds, coconuts, pecans, apples, pears, avocados, citrus, etc.). Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans, peas, and dry pulses. Beans include guar, locust bean, fenugreek, soybean, garden beans, cowpea, mungbean, lima bean, fava bean, lentils, chickpea, etc.

### A. Non-Limiting Plant Pests

In specific embodiments, the bacterial strains provided herein are those that target one or more plant pests. The term “pests” or “plant pests” includes but is not limited to insects, fungi, fungal-like organisms, bacteria, nematodes, viruses, viroids, protozoan pathogens, and the like. Plant pathogens include but are not limited to viruses, viroids, bacteria, insects which vector or spread plant diseases, nematodes, Oomycetes, plasmodiophorids, members of the Phytomyxea, fungi, fungal-like organisms, and the like. In specific embodiments, the bacterial strains, or active variants thereof, provided herein are those that target one or more plant pests. For example, any of the bacterial strain provided herein or an active variant thereof can have antifungal activity against one, two, three, four, five, or more fungal pathogens and/or fungal diseases described herein. Examples of plant diseases which can be treated or reduced or prevented include, but are not limited to, plant diseases caused by plant pathogens. Examples of such plant diseases include, but are not limited to, Asian Soybean Rust (ASR), gray mold, leaf spot, Frogeye Leaf Spot, Early Blight, Damping off complex, Brown Patch, black scurf, root rot, belly rot, Sheath Blight, Powdery Mildew, Anthracnose, Black Sigatoka, Anthracnose leaf spot, Downy Mildew, Pythium Blight, Late Blight, Fusarium Head Blight, sudden death syndrome (SDS), Fusarium Wilt, Corn Stalk Rot, Brown Rust, Black Rust, Yellow Rust, Wheat Rust, Rust, Apple Scab, Post-bloom Fruit Drop, Gummy Stem Blight, Greasy Spot, Corn Stalk Rot, Cherry Blossom Blight, Damping Off, Fire Blight, Citrus Greening Disease, Clubroot, Verticillium Wilt, Rhizopus Rot, Bacterial Spot, and Brown Rot, to name a few.

The methods and compositions disclosed herein can be used to control one or more fungal or fungal-like pathogens. In further embodiments, the bacterial strains or active variants thereof (i.e., AIP61892 or an active variant thereof, or a spore, or a forespore or a combination of cells, forespores and/or spores, and/or a composition derived from any one thereof) control at least one, two, three, four, five, or more fungal pathogens and/or fungal-like pathogens. A fungal pathogen can be, but is not limited to, a fungus selected from the group consisting of Aspergillus favus, Aspergillus spp., Albugo occidentalis, Albugo spp., Alternaria solani, Alternaria spp., Apiognomonia errabunda, Apiognomonia veneta, Apiognomonia spp., Armillaria mellea, Armillaria spp., Bipolaris maydis, Botrytis cinerea, Botrytis squamosa, Botrytis spp., Botryosphaeria dothidea, Botryosphaeira spp., Blumeriella jaapii, Blumeriella spp., Bremia lactucae, Bremia spp., Cladosporium carpophilum, Cladosporium caryigenum, Cladosporium spp., Colletotrichum acutatum, Colletotrichum graminicola, Colletotrichum cereale, Colletotrichum gloeosporiodes, Colletotrichum sublineolum, Colletotrichum spp, Cochhiobolus heterostrophus, Corynespora cassiicola, Corynespora spp., Disculafraxinea, Cercospora sojina, Cercospora beticola, Cercospora spp., Blumeria graminis f sp. Tritici, Didymella bryoniae, Didymella spp., Elsinoe fawcetti, Elsinoe spp., Erysiphe necator, Erysiphe lagerstroemiae, Erysiphe cichoracearum, Erysiphe spp., Eutypa lata, Eutypa spp., Fusarium graminearum, Fusarium solani, Fusarium oxysporum, Fusarium graminicola, Fusarium spp., Golovinomyces cichoracearum, Gibberella zeae, Gibberella spp., Gloeodes pomigena, Gymnosporangium juniperi-virginianae, Hemileia vastatrix, Leveillula taurica, Leveillula spp., Mycosphaerellafijiensis, Mycosphaerella citri, Mycosphaerella pomi, Mycosphaerella spp., Macrophomina spp., Monosporascus cannonballus, Monosporascus spp., Monilinia fructicola, Monilinia laxa, Monilinia fructigena, Monilinia spp., Neofabraea spp., Podosphaera xanthii, Podosphaera leucotricha, Podosphaera spp., Phomopsis viticola, Phomopsis spp., Penicillium spp., Phakopsora meibomiae, Phakopsora pachyrizi, Phakopsora spp., Puccinia triticina, Puccinia recondita, Puccinia striiformis, Puccinia graminis, Puccinia spp., Pyrenophora trici-repentis, Rhizoctonia solani, Rhizoctonia spp., Rhizopus spp., Rhizopus stolonifera, Ramularia spp., Tilletia barclayena, Tilletia spp., Uncinula necator, Uncinula spp., Uromyces betae, Uromyces spp., Phoma spp., Sclerotium rolfsii, Sclerotium spp., Sclerotinia minor, Sclerotinia sclerotiorium, Sclerotinia spp., Schizothyrium pomi, Schizothyrium spp., Septoria glycines, Septoria spp., Sphaerotheca pannosa, Sphaerotheca macularis, Sphaerotheca spp., Sphaceloma spp., Venturia inaequalis, Venturia spp., Verticillium spp., Wilsonomyces carpophilus, and Wilsonomyces spp.

In specific embodiments, fungal-like pathogens or fungal-like organisms refers to any organism that exhibits typical phenotypic characterisitics of fungi, but are not technically classified as fungi. In some embodiments, fungal-like pathogens were previously classified as fungi, but have changed classification. In specific embodiments Oomycetes, plasmodiophorids, and members of the Phytomyxea which are plant parasitic are referred to herein as fungal-like pathogens. Fungal-like pathogens can be, but are not limited to, Pythium cryptoirregulare, Pythium aphanidermatum, Pythium irregulare, Pythium sylvaticum, Pythium myriotylum, Pythium ultimum, Pythium spp., Phytophthora capsici, Phytophthora nicotianae, Phytophthora infestans, Phytophthora tropicalis, Phytophthora sojae, Phytophthora spp., Peronospora belbahrii, Peronospora lamii, Peronospora farinosa, Peronospora spp., Pseudoperonospora cubensis, Pseudoperonospora spp., Bremia spp., Plasmopara viticola, Plasmopara obduscens, Plasmopara spp., Basidiophora spp., Plasmodiophora brassicae, and Plasmodiophora spp.

In some embodiments, the fungal or fungal-like pathogen is selected from the group consisting of Aspergillus spp., Botrytis spp., Cercospora spp., Alternaria spp., Didymella spp., Fusarium spp., Erysiphe spp., Colletotrichum spp., Monilinia spp., Mycosphaerella spp., Plasmopara spp., Peronospora spp., Pythium spp., Phytophthora spp., Phomopsis spp., Phakopsora spp., Podosphaera spp., Rhizopus spp., Rhizoctonia spp., Sclerotium spp., Sclerotinia spp., Uncinula spp., Venturia spp., Wilsonomyces spp., and Plasmodiophora spp.

In further embodiments, the fungal or fungal-like pathogen is selected from the group consisting of Aspergillus flavus, Botrytis cinerea, Cercospora sojina, Alternaria solani, Colletotrichum acutatum, Colletotrichum cereal, Colletotrichum sublineolum, Didymella bryoniae, Erysiphe necator, Fusarium graminearum, Fusarium solani, Fusarium oxysporum, Monilinia fructicola, Monilinia laxa, Monilinia fructigena, Mycosphaerella citri, Mycosphaerella fijiensis, Podosphaera xanthii, Plasmopara viticola, Plasmodiophora brassicae, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Phakopsora pachyrizi, Rhizoctonia solani, Rhizopus stolonifera, Sclerotium rolfsii, Sclerotinia minor, Sclerotinia sclerotiorium, Uncinula necator, and Venturia inaequalis. In some embodiments, the bacterial strains or active variants thereof (i.e., AIP61892 or an active variant thereof, or a spore, or a forespore or a combination of cells, forespores and/or spores, and/or a composition derived from AIP61892, or an active variant of thereof) control at least one, two, three, four, five, or more fungal or fungal-like pathogens selected from the group consisting of Aspergillus flavus, Botrytis cinerea, Cercospora sojina, Alternaria solani, Colletotrichum acutatum, Colletotrichum cereal, Colletotrichum sublineolum, Didymella bryoniae, Erysiphe necator, Fusarium graminearum, Fusarium solani, Fusarium oxysporum, Monilinia fructicola, Monilinia laxa, Monilinia fructigena, Mycosphaerella citri, Mycosphaerella fijiensis, Podosphaera xanthii, Plasmopara viticola, Plasmodiophora brassicae, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Phakopsora pachyrizi, Rhizoctonia solani, Rhizopus stolonifera, Sclerotium rolfsii, Sclerotinia minor, Sclerotinia sclerotiorium, Uncinula necator, and Venturia inaequalis.

In some embodiments, the bacterial strains provided herein are those that control one or more bacterial pathogens. In further embodiments, the bacterial strains or active variants thereof (i.e., AIP61892 or an active variant thereof, or a spore, or a forespore or a combination of cells, forespores and/or spores, and/or a composition derived from any one thereof) control at least one, two, three, four, five, or more bacterial pathogens. Bacterial pathogens include Actinobacteria and Proteobacteria and are selected from the families of the Burkholderiaceae, Xanthomonadaceae, Pseudomonadaceae, Enterobacteriaceae, Microbacteriaceae, and Rhizobiaceae. A bacterial pathogen can be, but is not limited to, a bacterial species, sub-species, pathovar, or strain selected from the group consisting of [symbol] [symbol] Acidovorax avenae (=Pseudomonas avenae, Pseudomonas avenae subsp. avenae, Pseudomonas rubrilineans), including Acidovorax avenae subsp. avenae (=Pseudomonas avenae subsp. avenae), Acidovorax avenae subsp. cattleyae (=Pseudomonas cattleyae), Acidovorax avenae subsp. citrulli (=Pseudomonas pseudoalcaligene subsp. citrulli, Pseudomonas avenae subsp. citrulli)); Brenneria spp., Burkholderia spp., including Burkholderia andropogonis (=Pseudomonas andropogonis, Pseudomonas woodsii), Burkholderia caryophylli (=Pseudomonas caryophylli), Burkholderia cepacia (=Pseudomonas cepacia), Burkholderia gladioli (=Pseudomonas gladioli), Burkholderia gladioli pv. agaricicola (=Pseudomonas gladioli pv. agaricicola), Burkholderia gladioli pv. alliicola (=Pseudomonas gladioli pv. alliicola), Burkholderia gladioli pv. gladioli (=Pseudomonas gladioli, Pseudomonas gladioli pv. gladioli), Burkholderia glumae (=Pseudomonas glumae), Burkholderia plantarii (=Pseudomonas plantarii) Burkholderia solanacearum (=Ralstonia solanacearum), and Ralstonia spp.; Liberibacter spp., including Candidatus Liberibacter spp., including Liberibacter africanus (Laf), Liberibacter americanus (Lam), Liberibacter asiaticus (Las), Liberibacter europaeus (Leu), Liberibacter psyllaurous, Liberibacter solanacearum (Lso); Clavibacter spp. including Clavibacter michiganensis and Clavibacter sepedonicus; Corynebacterium, including Corynebacterium fascians, Corynebacterium flaccumfaciens pv. flaccumfaciens, Corynebacterium michiganensis, Corynebacterium michiganense pv. tritici, Corynebacterium michiganense pv. nebraskense, Corynebacterium sepedonicum; Dickeya spp. including Dickeya dadantii and Dickeya solani; Erwinia spp. including Erwinia amylovora, Erwinia ananas, Erwinia carotovora (=Pectobacterium carotovorum), Erwinia carotovora subsp. atroseptica, Erwinia carotovora subsp. carotovora, Erwinia chrysanthemi, Erwinia chrysanthemi pv. zeae, Erwinia dissolvens, Erwinia herbicola, Erwinia rhapontic, Erwinia stewartii, Erwinia tracheiphila, Erwinia uredovora; Pseudomonas savastanoi, Pseudomonas syringae, including Pseudomonas syringae pv. actinidiae (Psa), Pseudomonas syringae pv. atrofaciens, Pseudomonas syringae pv. coronafaciens, Pseudomonas syringae pv. glycinea, Pseudomonas syringae pv. lachrymans, Pseudomonas syringae pv. maculicola Pseudomonas syringae pv. papulans, Pseudomonas syringae pv. striafaciens, Pseudomonas syringae pv. syringae, Pseudomonas syringae pv. tomato, Pseudomonas syringae pv. tabaci; Pectobacterium spp., including Pectobacterium atrosepticum; Streptomyces spp. including Streptomyces acidiscabies, Streptomyces albidoflavus, Streptomyces candidus (=Actinomyces candidus), Streptomyces caviscabies, Streptomyces collinus, Streptomyces europaeiscabiei, Streptomyces intermedius, Streptomyces ipomoeae, Streptomyces luridiscabiei, Streptomyces niveiscabiei, Streptomyces puniciscabiei, Streptomyces retuculiscabiei, Streptomyces scabiei, Streptomyces scabies, Streptomyces setonii, Streptomyces steliiscabiei, Streptomyces turgidiscabies, Streptomyces wedmorensis; Xanthomonas axonopodis, including Xanthomonas axonopodis pv. alfalfae (=Xanthomonas alfalfae), Xanthomonas axonopodis pv. aurantifolii (=Xanthomonas fuscans subsp. aurantifolii), Xanthomonas axonopodis pv. allii (=Xanthomonas campestris pv. allii), Xanthomonas axonopodis pv. axonopodis, Xanthomonas axonopodis pv. bauhiniae (=Xanthomonas campestris pv. bauhiniae), Xanthomonas axonopodis pv. begonia (=Xanthomonas campestris pv. begoniae), Xanthomonas axonopodis pv. betlicola (=Xanthomonas campestris pv. betlicola), Xanthomonas axonopodis pv. biophyti (=Xanthomonas campestris pv. biophyti), Xanthomonas axonopodis pv. cajani (=Xanthomonas campestris pv. cajani), Xanthomonas axonopodis pv. cassava (=Xanthomonas cassavae, Xanthomonas campestris pv. cassavae), Xanthomonas axonopodis pv. cassiae (=Xanthomonas campestris pv. cassiae), Xanthomonas axonopodis pv. citri (=Xanthomonas citri), Xanthomonas axonopodis pv. citrumelo (=Xanthomonas alfalfa subsp. citrumelonis), Xanthomonas axonopodis pv. clitoriae (=Xanthomonas campestris pv. clitoriae), Xanthomonas axonopodis pv. coracanae (=Xanthomonas campestris pv. coracanae), Xanthomonas axonopodis pv. cyamopsidis (=Xanthomonas campestris pv. cyamopsidis), Xanthomonas axonopodis pv. desmodii (=Xanthomonas campestris pv. desmodii), Xanthomonas axonopodis pv. desmodiigangetici (=Xanthomonas campestris pv. desmodiigangetici), Xanthomonas axonopodis pv. desmodiilaxiflori (=Xanthomonas campestris pv. desmodiilaxiflori), Xanthomonas axonopodis pv. desmodiirotundifolii (=Xanthomonas campestris pv. desmodiirotundifolii), Xanthomonas axonopodis pv. dieffenbachiae (=Xanthomonas campestris pv. dieffenbachiae), Xanthomonas axonopodis pv. erythrinae (=Xanthomonas campestris pv. erythrinae), Xanthomonas axonopodis pv. fascicularis (=Xanthomonas campestris pv. fasciculari), Xanthomonas axonopodis pv. glycines (=Xanthomonas campestris pv. glycines), Xanthomonas axonopodis pv. khayae (=Xanthomonas campestris pv. khayae), Xanthomonas axonopodis pv. lespedezae (=Xanthomonas campestris pv. lespedezae), Xanthomonas axonopodis pv. maculifoliigardeniae (=Xanthomonas campestris pv. maculifoliigardeniae), Xanthomonas axonopodis pv. malvacearum (=Xanthomonas citri subsp. malvacearum), Xanthomonas axonopodis pv. manihotis (=Xanthomonas campestris pv. manihotis), Xanthomonas axonopodis pv. martyniicola (=Xanthomonas campestris pv. martyniicola), Xanthomonas axonopodis pv. melhusii (=Xanthomonas campestris pv. melhusii), Xanthomonas axonopodis pv. nakataecorchori (=Xanthomonas campestris pv. nakataecorchori), Xanthomonas axonopodis pv. passiflorae (=Xanthomonas campestris pv. passiflorae), Xanthomonas axonopodis pv. pateli (=Xanthomonas campestris pv. patelii), Xanthomonas axonopodis pv. pedalii (=Xanthomonas campestris pv. pedalii), Xanthomonas axonopodis pv. phaseoli (=Xanthomonas campestris pv. phaseoli, Xanthomonas phaseoli), Xanthomonas axonopodis pv. phaseoli var. fuscans (=Xanthomonas fuscans), Xanthomonas axonopodis pv. phyllanthi (=Xanthomonas campestris pv. phyllanthi), Xanthomonas axonopodis pv. physalidicola (=Xanthomonas campestris pv. physalidicola), Xanthomonas axonopodis pv. poinsettiicola (=Xanthomonas campestris pv. poinsettiicola), Xanthomonas axonopodis pv. punicae (=Xanthomonas campestris pv. punicae), Xanthomonas axonopodis pv. rhynchosiae (=Xanthomonas campestris pv. rhynchosiae), Xanthomonas axonopodis pv. ricini (=Xanthomonas campestris pv. ricini), Xanthomonas axonopodis pv. sesbaniae (=Xanthomonas campestris pv. sesbaniae), Xanthomonas axonopodis pv. tamarindi (=Xanthomonas campestris pv. tamarindi), Xanthomonas axonopodis pv. vasculorum (=Xanthomonas campestris pv. vasculorum), Xanthomonas axonopodis pv. vesicatoria (=Xanthomonas campestris pv. vesicatoria, Xanthomonas vesicatoria), Xanthomonas axonopodis pv. vignaeradiatae (=Xanthomonas campestris pv. vignaeradiatae), Xanthomonas axonopodis pv. vignicola (=Xanthomonas campestris pv. vignicola), Xanthomonas axonopodis pv. vitians (=Xanthomonas campestris pv. vitians); Xanthomonas campestris pv. musacearum, Xanthomonas campestris pv. pruni (=Xanthomonas arboricola pv. pruni), Xanthomonas fragariae; Xanthomonas oryzae, Xanthomonas translucens (=Xanthomonas campestris pv. hordei) including Xanthomonas translucens pv. arrhenatheri (=Xanthomonas campestris pv. arrhenatheri), Xanthomonas translucens pv. cerealis (=Xanthomonas campestris pv. cerealis), Xanthomonas translucens pv. graminis (=Xanthomonas campestris pv. graminis), Xanthomonas translucens pv. phlei (=Xanthomonas campestris pv. phlei), Xanthomonas translucens pv. phleipratensis (=Xanthomonas campestris pv. phleipratensis), Xanthomonas translucens pv. poae (=Xanthomonas campestris pv. poae), Xanthomonas translucens pv. secalis (=Xanthomonas campestris pv. secalis), Xanthomonas translucens pv. translucens (=Xanthomonas campestris pv. translucens), Xanthomonas translucens pv. undulosa (=Xanthomonas campestris pv. undulosa), Xanthomonas oryzae, Xanthomonas oryzae pv. oryzae (=Xanthomonas campestris pv. oryzae), Xanthomonas oryzae pv. oryzicola (=Xanthomonas campestris pv. oryzicola), and Xylella fastidiosa from the family of Xanthomonadaceae.

In some embodiments, the bacterial pathogen is a species, sub-species, pathovar, or strain selected from the group consisting of Acidovorax avenae, Burkholderia gladioli, Candidatus Liberibacter spp., Erwinia amylovora, Erwinia ananas, Erwinia carotovora, Erwinia chrysanthemi, Erwinia dissolvens, Erwinia herbicola, Erwinia rhapontic, Erwinia stewartii, Erwinia tracheiphila, Erwinia uredovora; Pseudomonas syringae, Streptomyces scabies, Xanthomonas campestris, Xanthomonas axonopodis, Xanthomonas fragariae; Xanthomonas translucens, and Xylella fastidiosa. In some embodiments, the bacterial strains or active variants thereof (i.e., AIP61892 or an active variant thereof, or a spore, or a forespore or a combination of cells, forespores and/or spores, and/or a composition derived from AIP61892, or an active variant of thereof) control at least one, two, three, four, five, or more bacterial pathogens selected from the group consisting of a species, sub-species, pathovar, or strain of Acidovorax avenae, Burkholderia gladioli, Candidatus Liberibacter spp., Erwinia amylovora, Erwinia ananas, Erwinia chrysanthemi, Erwinia dissolvens, Erwinia herbicola, Erwinia rhapontic, Erwinia stewartii, Erwinia tracheiphila, Erwinia uredovora; Pectobacterium carotovorum, Pseudomonas syringae, Streptomyces scabies, Xanthomonas campestris, Xanthomonas axonopodis, Xanthomonas fragariae; Xanthomonas translucens, and Xylella fastidiosa.

In some embodiments, the bacterial strains provided herein are those that control one or more insect or insect pests. In further embodiments, the bacterial strains or active variants thereof (i.e., AIP61892 or an active variant thereof, or a spore, or a forespore or a combination of cells, forespores and/or spores, and/or a composition derived from any one thereof) control at least one, two, three, four, five, or more insects and/or insect pests. The term “insects” or “insect pests” as used herein refers to insects and other similar pests. The insect or insect pest may either feed from a plant tissue, such as a leaf, fruit, stalk, or root, or it may pierce a plant tissue and feed on plant fluids, such as the phloem. The insect or insect pest may act as a vector for plant pathogens, for example for viral or bacterial plant pathogens. The term “insect” encompasses eggs, larvae, juvenile and mature forms of insects. Insects can be targeted at any stage of development. For example, insects can be targeted after the first instar, during the second instar, third instar, fourth instar, fifth instar, or any other developmental or adult growth stage. As used herein, the term “instar” is used to denote the developmental stage of the larval or nymphal forms of insects. Insect pests include insects selected from the orders Acari, Coleoptera, Lepidoptera, Hemiptera, Diptera, Hymenoptera, Lepidoptera, Mallophaga, Homoptera, Hemiptera, Orthroptera, Thysanoptera, Trombidiformes, Dermaptera, Isoptera, Anoplura, Siphonaptera, and Trichoptera.

Insect pests of the order Coleoptera include, but are not limited to, Agriotes spp., Anthonomus spp., Atomaria linearis, Chaetocnema tibialis, Cosmopolites spp., Curculio spp., Dermestes spp., Epilachna spp., Eremnus spp., Leptinotarsa decemlineata, Lissorhoptrus spp., Melolontha spp., Orycaephilus spp., Otiorhynchus spp., Phlyctinus spp., Popillia spp., Psylliodes spp., Rhizopertha spp., Scarabeidae, Sitophilus spp., Sitotroga spp., Tenebrio spp., Tribolium spp., and Trogoderma spp. In specific embodiments, Coleoptera insects include, but are not limited to weevils from the families Anthribidae, Bruchidae, and Curculionidae (e.g., sweetpotato weevil (Cylas formicarius (Fabricius)), boll weevil (Anthonomus grandis Boheman), rice water weevil (Lissorhoptrus oryzophilus Kuschel), rice weevil (Sitophilus oryzae L.)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scaribaeidae (e.g., Japanese beetle (Popillia japonica Newman) and European chafer (Rhizotrogus majalis Razoumowsky)); wireworms from the family Elateridae and bark beetles from the family Scolytidae.

As disclosed herein, insect pests include Coleoptera pests of the corn rootworm complex: Western corn rootworm, Diabrotica virgifera virgifera; northern corn rootworm, D. barberi; Southern corn rootworm or spotted cucumber beetle, Diabrotica undecimpunctata howardi; and the Mexican corn rootworm, D. virgifera zeae. In specific embodiments, the insect pest is Western corn rootworm, Colorado Potato Beetle, and/or sweet potato weevil.

Insect pests that can be controlled with the compositions and methods disclosed herein further include insects of the order Lepidoptera, including Achoroia grisella, Acleris gloverana, Acleris variana, Adoxophyes orana, Agrotis ipsilon, Alabama argillacea, Alsophila pometaria, Amyelois transitella, Anagasta kuehniella, Anarsia lineatella, Anisota senatoria, Antheraea pernyi, Anticarsia gemmatalis, Archips spp., Argyrotaenia spp., Athetis mindara, Bombyx mori, Bucculatrix thurberiella, Cadra cautella, Choristoneura sp., Cochylls hospes, Colias eurytheme, Corcyra cephalonica, Cydia latiferreanus, Cydia pomonella, Datana integerrima, Dendrolimus sibericus, Desmiafeneralis spp., Diaphania hyalinata, Diaphania nitidalis, Diatraea grandiosella, Diatraea saccharalis, Ennomos subsignaria, Eoreuma loftini, Esphestia elutella, Erannis tilaria, Estigmene acrea, Eulia salubricola, Eupocoellia ambiguella, Eupoecilia ambiguella, Euproctis chrysorrhoea, Euxoa messoria, Galleria mellonella, Grapholita molesta, Harrisina americana, Helicoverpa subflexa, Helicoverpa zea, Heliothis virescens, Hemileuca oliviae, Homoeosoma electellum, Hyphantia cunea, Keiferia lycopersicella, Lambdina fiscellaria fiscellaria, Lambdina fscellaria lugubrosa, Leucoma salicis, Lobesia botrana, Loxostege sticticalis, Lymantria dispar, Macalla thyrisalis, Malacosoma spp., Mamestra brassicae, Mamestra configurata, Manduca quinquemaculata, Manduca sexta, Maruca testula/is, Melanchra picta, Operophtera brumata, Orgyia spp., Ostrinia nubila/is, Paleacrita vernata, Papilio cresphontes, Pectinophora gossypiella, Phryganidia californica, Phyllonorycter blancardella, Pieris napi, Pieris rapae, Plathypena scabra, Platynota flouendana, Platynota stultana, Platyptilia carduidactyla, Plodia interpunctella, Plutella xylostella, Pontia protodice, Pseudaletia unipuncta, Pseudoplasia includens, Sabulodes aegrotata, Schizura concinna, Sitotroga cerealella, Spilonta ocellana, Spodoptera spp. including Spodoptera frugiperda, Thaurnstopoea pityocampa, Tinsola bisselliella, Trichoplusia hi, Tuta absoluta, Udea rubigalis, Xylomyges curiails, and Yponomeuta padella.

The methods and compositions provided herein can also be used against insect pests of the order Hemiptera including, but not limited to, Lygus spp., including Lygus spp. including Lygus hesperus, Lygus lineolaris, Lygus pratensis, Lygus rugulipennis, and Lygus pabulinus, Calocoris norvegicus, Orthops compestris, Plesiocoris rugicollis, Cyrtopeltis modestus, Cyrtopeltis notatus, Spanagonicus albofasciatus, Diaphnocoris chlorinonis, Labopidicola alloi, Pseudatomoscelis seriatus, Adelphocoris rapidus, Poecilocapsus lineatus, Blissus leucopterus, Nysius spp. including Nysius ericae and Nysius raphanus, Nezara viridula, Acrosternum hilare, Euschistus spp. including Euschistus servus and Euschistus heros, Dichelops spp. including Dichelops melacantus and Dichelops furcatus, Halyomorpha halys, Lipaphis erysimi, Aphis gossypii, Macrosiphum avenae, Myzus persicae, Acyrthosiphon pisum, Aphidoidea spp, Eurygaster spp., Coreidae spp., Pyrrhocoridae spp., Blostomatidae spp., Reduviidae spp., Cimicidae spp., Aleurocanthus woglumi, Aleyrodes proletella, Bemisia spp. including Bemisia argentifolii and Bemisia tabaci, Trialeurodes vaporariorum, and psyllids including Diaphorina spp. including Diaphorina citri and Trioza spp. including Trioza erytreae.

The methods and compositions provided herein can also be used against insect pests of the order Thysenoptera including, but not limited to, thrips species, including Frankliniella spp., for example Western Flower thrips (Frankliniella occidentalis (Pergande)); Thrips spp., for example Thrips tabaci; Scirtothrips spp., for example Scirtothrips dorsalis; Klambothrips spp., for example Klambothrips myopori; Echinothrips spp., for example Echinothrips americanus; and Megalurothrips spp., for example Megalurothrips usitatus.

The methods and compositions provided herein can also be used against insect pests of the order Trombidiformes including, but are not limited to, plant feeding mites, including six-spooted spider mite (Eutetranychus sexmaculatus), Texas citrus mite (Eutetranychus banksi), Citrus red mite (Panonychus citri), European red mite (Panonychus ulmi), McDaniel mite (Tetranychus mcdanieli), Pacific spider mite (Tetranychus pacificus), Strawberry spider mite (Tetranychus urticae), Spruce spider mite (Oligonychus ununguis), Sugi spider mite (Oligonychus nondonensisi), and Tetranychus evansi.

In specific embodiments, the bacterial strains provided herein are those that control one or more insect or insect pests. For example, the various bacterial strains provided herein target one or more insect pests that cause damage to plants. For example, any of the bacterial strain provided herein or an active variant thereof can have insecticidal activity against one, two, three, four, five, or more insect pests described herein.

In some embodiments, the compositions and methods provided herein control nematode plant pests. Nematodes include parasitic nematodes such as root-knot, cyst, and lesion nematodes, including of the species Meloidogyne such as the Southern Root-Knot nematode (Meloidogyne incognita), Javanese Root-Knot nematode (Meloidogyne javanica), Northern Root-Knot Nematode (Meloidogyne hapla) and Peanut Root-Knot Nematode (Meloidogyne arenaria); nematodes of the species Ditylenchus such as Ditylenchus destructor and Ditylenchus dipsaci; nematodes of the species Pratylenchus such as the Cob Root-Lesion Nematode (Pratylenchus penetrans), Chrysanthemum Root-Lesion Nematode (Pratylenchus fallax), Pratylenchus coffeae, Pratylenchus loosi and Walnut Root-Lesion Nematode (Pratylenchus vulnus); Nematodes of the species Globodera such as Globodera rostochiensis and Globodera pallida; Nematodes of the species Heterodera such as Heterodera glycines (soybean cyst nematode); Heterodera schachtii (beet cyst nematode); Heterodera avenae (cereal cyst nematode); Nematodes of the species Aphelenchoides such as the Rice White-tip Nematode (Aphelenchoides besseyi), Aphelenchoides ritzemabosi and Aphelenchoides fragariae; Nematodes of the species Aphelenchus such as Aphelenchus avenae; Nematodes of the species Radopholus, such as the Burrowing-Nematode (Radopholus similis); Nematodes of the species Tylenchulus such as Tylenchulus semipenetrans; Nematodes of the species Rotylenchulus such as Rotylenchulus reniformis; Nematodes living in trees such as Bursaphelenchus xylophilus and the Red Ring Nematode (Bursaphelenchus cocophilus) etc. and Globodera spp.; particularly members of the cyst nematodes, including, but not limited to Globodera rostochiensis and Globodera pailida (potato cyst nematodes); Spiral (Helicotylenchus spp.); Burrowing (Radopholus similis); Bulb and stem (Ditylenchus dipsaci); Reniform (Rotylenchulus reniformis); Dagger (Xiphinema spp.); Bud and leaf (Aphelenchoides spp.); and Pine Wilt Disease (Bursaphelenchus xylophilus). Lesion nematodes include Pratylenchus spp. The term “nematode” encompasses eggs, larvae, juvenile and mature forms of nematodes.

Bacterial strains or active variants thereof and/or a composition derived therefrom can be tested for pesticidal activity against a pest in any developmental stage, including early developmental stages, e.g., as larvae or other immature forms. For example, larvae of insect pests may be reared in total darkness at from about 20° C. to about 30° C. and from about 30% to about 70% relative humidity. Bioassays may be performed as described in Czapla and Lang (1990) J. Econ. Entomol. 83 (6): 2480-2485. Methods of rearing insect larvae and performing bioassays are well known to one of ordinary skill in the art.

In specific embodiments, the bacterial strains provided herein are those that control one or more nematode or nematode pests. For example, the various bacterial strains provided herein control one or more nematode pests that cause damage to plants. For example, any of the bacterial strain provided herein or an active variant thereof can have nematicidal activity against one, two, three, four, five, or more nematode pests described herein.

In one non-limiting embodiment, the bacterial strain, active variant thereof, and/or a composition derived therefrom provided herein can be employed to decrease or reduce the level of a plant pest. The term “pests” includes but is not limited to, insects, fungi, fungal-like organisms, bacteria, nematodes, viruses, viroids, protozoan pathogens, and the like. By “pest resistance” is intended that the bacterial strain, active variant thereof, and/or a composition derived therefrom provided herein can inhibit (inhibit growth, feeding, fecundity, or viability), suppress (suppressing growth, feeding, fecundity, or viability), reduce (reduce the pest infestation, reduce the pest feeding activities on a particular plant) or kill (cause the morbidity, mortality, or reduced fecundity of) a pest, such as an insect pest. By “a plant susceptible to a pest” is meant that a pest is able to infect or damage the plant. For example, a plant susceptible to a pest can be susceptible to damage caused by a insect, or nematode pest as disclosed elsewhere herein.

In further embodiments, the bacterial strains or active variants thereof (i.e., AIP61892 or an active variant thereof, or a spore, or a forespore or a combination of cells, forespores and/or spores, and/or a composition derived from any one of AIP61892 or an active variant thereof) control at least one, two, three, four, five, or more of the plant pathogens and/or plant pests described herein. In specific embodiments, the bacterial strains AIP61892 or active variants thereof, or an active variant thereof, or a spore, or a forespore or a combination of cells, forespores and/or spores, and/or a composition derived from any one of AIP61892 or an active variant thereof control at least one plant pest, plant pathogen, and/or plant disease described herein.

In further embodiments, the bacterial strains or active variants thereof (i.e., AIP61892 or an active variant thereof, or a spore, or a forespore or a combination of cells, forespores and/or spores, and/or a composition derived from any one of AIP61892 or an active variant thereof) control at least one, two, three, four, five, or more plant diseases or diseases caused by plant pathogens or plant pests such as insect pests, selected from the group consisting of Asian Soybean Rust (ASR), gray mold, leaf spot, Frogeye Leaf Spot, Early Blight, Damping off complex, Brown Patch, black scurf, root rot, belly rot, Sheath Blight, Powdery Mildew, Anthracnose, Black Sigatoka, Anthracnose leaf spot, Downy Mildew, Pythium Blight, Late Blight, Fusarium Head Blight, sudden death syndrome (SDS), Fusarium Wilt, Corn Stalk Rot, Brown Rust, Black Rust, Yellow Rust, Wheat Rust, Rust, Apple Scab, Post-bloom Fruit Drop, Gummy Stem Blight, Greasy Spot, Corn Stalk Rot, Cherry Blossom Blight, Damping Off, Fire Blight, Citrus Greening Disease, Clubroot, Verticillium Wilt, Rhizopus Rot, Bacterial Spot, and Brown Rot.

### B. Methods of Treating or Preventing Plant Disease

Provided herein are methods of treating or preventing a plant disease comprising applying to a plant having a plant disease or at risk of developing a plant disease an effective amount of bacterial strain AIP61892 or an active variant thereof (and in some embodiments, a copper compound or AIP1620 or variant thereof), and/or a composition derived therefrom wherein the bacterial strain controls a plant pest that causes the plant disease. The plant pest may be a virus, viroid, bacteria, nematode, fungus, fungal-like organism (such as and including an Oomycete, plasmodiophorid, and a member of the Phytomyxea), insect, or protozoan pathogen. Also provided herein are methods of controlling a plant pest or preventing plant damage caused by a plant pest comprising applying to a plant an effective amount of bacterial strain AIP61892 or an active variant thereof (and in some embodiments, a copper compound or AIP1620 or variant thereof), and/or a composition derived therefrom wherein the bacterial strain controls a plant pest. Also provided herein are methods of reducing susceptibility to a plant pest and/or increasing resistance to a plant pest comprising applying to a plant having a plant disease or damage or at risk of developing a plant disease or damage caused by a plant pest an effective amount of bacterial strain AIP61892 or an active variant thereof (and in some embodiments, a copper compound or AIP1620 or variant thereof), and/or a composition derived therefrom wherein the bacterial strain controls the plant pest. In certain embodiments, the bacterial strain provided herein or an active variant thereof, and/or a composition derived therefrom may comprise at least one of AIP61892, or an active variant thereof, or a spore, or a forespore or a combination of cells, forespores and/or spores from any one of AIP61892, or an active variant or any thereof. In some embodiments, the effective amount of the bacterial strain or an active variant thereof and/or a composition derived therefrom comprises at least about 10⁴ to 10¹⁶ CFU per hectare, at least about 10¹² to 10¹⁶ CFU per hectare, or least about 10⁵ to 10¹¹ CFU per hectare. In some embodiments, the composition is derived from a bacterial strain provided herein or an active variant thereof which may comprise a cell of at least one of AIP61892 or an active variant thereof, or a spore, or a forespore or a combination of cells, forespores and/or spores from any one of AIP61892 or an active variant thereof.

In some methods, the bacterial strain provided herein or an active variant therof, and/or a composition derived therefrom is an agent that treats or prevents one, two, three, four, five or more plant diseases, infections, or infestations by plant pests. In other methods, the bacterial strain provided herein or an active variant thereof, and/or a composition derived therefrom is an antipesticidal agent that treats or prevents one, two, three, four, five or more fungal plant diseases, diseases caused by fungal-like pathogens, diseases caused by bacterial pathogens, or infections or infestations caused by insect pests or nematode pests. The bacterial strain provided herein or an active variant therof, and/or a composition derived therefrom can be employed with any plant species susceptible to a plant disease of interest and/or susceptible to a plant pest of interest.

Examples of diseases causes by fungal, fungal-like, bacterial, or other plant pests described herein are provided in Table 1. Also provided are non-limiting exemplary crop species that are susceptible to the plant diseases caused by the pathogens. For example, Table 1 shows that Bortrytis cinerea causes gray mold on all flowering crops. Therefore, a bacterial strain provided herein or active variant therof, and/or a composition derived therefrom that controls Bortrytis cinerea can be applied to a plant having gray mold or at risk of developing gray mold in order to treat or prevent gray mold in the plant. Similarly, Table 1 shows that Rhizoctonia solani causes Damping off complex in corn, Damping off complex in soybean, Brown Patch in turf, and Damping off complex in ornamentals. Therefore, a bacterial strain provided herein or an active variant thereof, and/or a composition derived therefrom that controls Rhizoctonia solani can be applied to a plant having Damping off complex and/or brown patch or at risk of developing Damping off complex and/or brown patch in order to treat or prevent Damping off complex and/or brown patch in the plant. In yet another example, Table 1 shows that Colletotrichum cereale, Apiognomonia errabunda, Apiognomonia veneta, Colletotrichum gloeosporiodes, Discula fraxinea cause Anthracnose leaf spot. Therefore, a bacterial strain provided herein or an active variant thereof, and/or a composition derived therefrom that controls one or more of Colletotrichum cereale, Apiognomonia errabunda, Apiognomonia veneta, Colletotrichum gloeosporiodes, Disculafraxinea can be applied to a plant having Anthracnose leaf spot or at risk of developing Anthracnose leaf spot in order to treat or prevent Anthracnose leaf spot in the plant.

TABLE 1

| Causal Pathogen | Disease | Crop-species |
|---|---|---|
| Botrytis cinerea | gray mold | all flowering crops |
| Cersospora spp | Leaf Spot | Ornamentals |
| Cercospora sojina | Frogeye leaf spot | Soybeans |
| Cercospora beticola | Cercospora leaf spot | beets, spinach, chard |
| Alternaria solani | Early Blight | solanaceous plants |
| Alternaria spp. | Pin Rot | Brassica |
| Rhizoctonia solani | Damping off complex | Corn, soybean, |
| | | ornamentals |
| Rhizoctonia solani | Brown Patch | Turf |
| Rhizoctonia solani | black scurf | potato |
| Rhizoctonia solani | root rot | sugar beet |
| Rhizoctonia solani | belly rot | cucurbit |
| Rhizoctonia solani | sheath blight | rice |
| Blumeria graminis f. sp. Tritici | Powdery Mildew | Wheat |
| Erysiphe spp. | Powdery Mildew | Grape, Brassica, |
| | | ornamentals |
| Podosphaera xanthii | Powdery Mildew | Cucurbit |
| Golovinomyces cichoracearum | Powdery Mildew | Ornamentals |
| Sphaerotheca pannosa | Powdery Mildew | Ornamentals |
| Colletotrichum cereale | Anthracnose leaf spot | Turf/grasses/cereal |
| Apiognomonia errabunda | Anthracnose leaf spot | Turf/grasses/cereal |
| Apiognomonia veneta | Anthracnose leaf spot | Turf/grasses/cereal |
| Colletotrichum gloeosporiodes | Anthracnose leaf spot | Turf/grasses/cereal |
| Discula fraxinea | Anthracnose leaf spot | Turf/grasses/cereal |
| Plasmopara viticola | Downy Mildew | Grape |
| Pseudoperonospora cubensis | Downy Mildew | Cucurbit |
| Peronospora belbahrii | Downy Mildew | Basil |
| Bremia lactucae | Downy Mildew | Lettuce |
| Peronospora lamii | Downy Mildew | Coleus |
| Plasmopara obduscens | Downy Mildew | Impatiens |
| Pythium cryptoirregulare | Damping off complex | Ornamental Plants |
| Pythium aphanidermatum | Pythium Blight/Damping off | turf/ornamentals/row |
| | complex | crop |
| Pythium irregulare | Damping off complex | turf/ornamentals/row |
| | | crop |
| Pythium sylvaticum | Damping off complex | turf/ornamentals/row |
| | | crop |
| Pythium myriotylum | Damping off complex | turf/ornamentals/row |
| | | crop |
| Pythium ultimum | Pythium Blight/Damping off | turf/ornamentals/row |
| | complex | crop |
| Phytophthora capsici | Phytophthora blight | cucurbit/pepper |
| Phytophthora nicotianae | Black shank | ornamental plants, |
| | | ctoon, peeper, citrus, |
| | | onion |
| Phytophthora infestans | Late Blight | solanaceous plants |
| Phytophthora tropicalis | | ornamental |
| | | plants/peppers/tropical |
| | | nut trees |
| Phytophthora sojae | | Soybean |
| Fusarium graminearum | Fusarium Head Blight | Cereals-Wheat |
| Fusarium solani | SDS | Soybean |
| Fusarium oxysporum | Fusarium Wilt | Herbaceous Plants |
| Fusarium graminicola | Corn Stalk Rot | Maize |
| Gibberella zeae | Corn Stalk Rot | Maize |
| Colletotrichum graminicola | Corn Stalk Rot | Maize |
| Phakopsora pachyrizi | Asian Soybean Rust | Soybean |
| Puccinia triticina | Brown Rust | Cereals |
| Puccinia recondita | Black Rust | Cereals |
| Puccinia striiformis | Yellow Rust | Cereals |
| Puccinia graminis | Wheat Rust | Cereals |
| Puccinia spp. | Rust | Ornamentals |
| Venturia inaequalis | Apple Scab | Malus |
| Verticillium spp | Verticillium Wilt | All |
| Monilinia fructicola | Brown Rot | Stone Fruits |
| Monilinia laxa | Brown Rot | Stone Fruits |
| Monilinia fructigena | Brown Rot | Stone Fruits |
| Erwinia amylovora | Fire Blight | Rosacea family, Pome |
| | | Fruit. |
| Erwinia chrysanthemi | Bacterial Crown Rot | Artichokes |
| Erwinia spp. | Bacterial Rot | Brassica |
| Erwinia spp. | Bacterial Canker | Papaya |
| Erwinia carotovora | Aerial Stem Rot | Root and Tuber |
| | | Vegetables |
| Xanthomonas campestris | Black Rot, Xanthomonas Leaf | Brassicas |
| | Spot | |
| Xanthomonas campestris | Walnut Blight | Nut Trees |
| Xanthomonas campestris | Bacterial Canker | Avocado, Mango |
| Xanthomonas spp. | Xanthomonas Leaf Blight | Bulb Vegetables |
| Xanthomonas spp. | Bacterial Blight and Bacterial | Cereal Grains, Cotton |
| | Streak | |
| Xanthomonas spp. | Citrus Canker | Citrus |
| Xanthomonas spp. | Bacterial Spot | Fruiting Vegetables |
| Xanthomonas spp. | Bacterial Leaf Blight/Bacterial | Leafy Vegetables, |
| | Leaf Spot | Root and Tuber |
| | | Vegetables |
| Xanthomonas spp. | Bacterial Pustule | Legume Vegetables, |
| | | Oilseed Crops, |
| | | Soybean |
| Xanthomonas spp. | Bacterial Leaf Spot/Bacterial Spot | Stone Fruit, Nut Trees |
| Xanthomonas fragariae | Angular Leaf Spot | Strawberry |
| Xanthomonas spp. | Gumming Disease | Sugarcane |
| Pseudomonas spp. | Bacterial Canker | Berry, Stone Fruit, |
| Pseudomonas spp. | Bacterial Leaf Spot and Bacterial | Brassica, Kiwi, |
| | Blight | |
| Pseudomonas spp. | Bacterial Leaf Streak | Bulb Vegetables |
| Pseudomonas spp. | Bacterial Speck | Oilseed Crops |
| Pseudomonas syringae | Bacterial Blast | Citrus |
| Pseudomonas syringae | Bacterial Blight | Coffee. herbs, spices |
| Pseudomonas syringae | Angular Leaf Spot | Cucurbits |
| Pseudomonas syringae | Bacterial Canker | Nut Trees |
| Pseudomonas syringae | Spinach Bacterial Leaf Spot | Leafy Vegetables |
| Pseudomonas syringae pv. tomato | Bacterial Speck | Fruiting Vegetables |
| Pseudomonas savastanoi | Olive Knot | Olive |
| Candidatus Liberibacter spp. | Citrus Greening Disease | citrus |
| Plasmodiophora brassicae | Club Root Disease | Brassicas, Oilseed |
| | | Crops, Root and |
| | | Tuber Vegetables, |
| Rhizopus stolonifera | Rhizopus Rot | Stone fruits |
| Uncinula necator | Powdery Mildew | Grape |

The term “treat” or “treating” or its derivatives includes substantially inhibiting, slowing, or reversing the progression of a condition, substantially ameliorating symptoms of a condition or substantially preventing the appearance of symptoms or conditions brought about by the pathogen or pest that causes the plant disease.

The terms “controlling” and “protecting a plant from a pest” or “protecting a plant from a pathogen” refers to one or more of inhibiting or reducing the growth, germination, reproduction, and/or proliferation of a pathogen of interest; and/or killing, removing, destroying, or otherwise diminishing the occurrence, and/or activity of a pathogen of interest. As such, a plant or plant part treated with the bacterial strain provided herein may show a reduced disease severity or reduced disease development in the presence of plant pathogens by a statistically significant amount. The bacterial strains, or combinations thereof, provided herein can reduce the growth, germination, reproduction, and/or proliferation of a pathogen of interest on a plant or plant part in a field or area of cultivation or following removal of the plant or plant part from a field or area of cultivation.

The term “prevent” and is variations means the countering in advance of bacterial, fungal, viral, insect or other pest growth, proliferation, infestation, spore germination, and hyphae growth. In this instance, the composition is applied before exposure to the pathogens or plant pests.

The term “ameliorate” and “amelioration” relate to the improvement in the treated plant condition brought about by the compositions and methods provided herein. The improvement can be manifested in the forms of a decrease in pathogen or pest growth and/or an improvement in the diseased plant height, weight, number of leaves, root system, or yield. In general, the term refers to the improvement in a diseased plant physiological state.

The term “inhibit” and all variations of this term is intended to encompass the restriction or prohibition of bacterial, fungal, viral, nematode, insect, or any other pest growth, as well as spore germination.

The term “eliminate” relates to the substantial eradication or removal of bacteria, fungi, viruses, nematodes, insects, or any other pests by contacting them with the composition of the invention, optionally, according to the methods of the invention described below.

The terms “delay”, “retard” and all variations thereof are intended to encompass the slowing of the progress of bacterial, fungal, viral, nematode, insect, or any other pest growth, and spore germination. The expression “delaying the onset” is interpreted as preventing or slowing the progression of bacterial, fungal, viral, nematodes, insect, or any other pest growth, infestation, infection, spore germination and hyphae growth for a period of time, such that said bacterial, fungal, viral, nematode, insect, or any other pest growth, infestation, infection, spore germination and hyphae growth do not progress as far along in development, or appear later than in the absence of the treatment according to the invention.

A plant, plant part, or area of cultivation treated with the bacterial strain provided herein or an active variant thereof, and/or a composition derived therefrom may show a reduced disease severity or reduced disease development in the presence of plant pathogens or plant pest by a statistically significant amount. A reduced disease severity or reduced disease development can be a reduction of about 10% to about 20%, about 20% to about 30%, about 30% to about 40%, about 40% to about 50%, about 50% to about 60%, about 60% to about 70%, about 70% to about 80%, about 80% to about 90%, or about 90% to about 100% when compared to non-treated control plants. In other instances, the plant treated with a bacterial strain provided herein or an active variant thereof, and/or a composition derived therefrom may show a reduced disease severity or reduced disease development in the presence of plant pathogen of at least about 10%, 11%, 12%, 130%, 14%, 150%, 16%, 17%, 180%, 19%, 20%, 210%, 22%, 230%, 24%, 250%, 26%, 27%, 280%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% greater when compared to non-treated control plants. Methods for assessing plant disease severity are known, and include, measuring percentage of diseased leaf area (Godoy et al. (2006) Fitopatol. Bras. 31(1) 63-68 or by measuring growth of the pathogen, for example uredinia counts for Phakopsora pachyrhiz (see Example 5).

A plant, plant part, or area of cultivation treated with the bacterial strain provided herein or an active variant thereof, and/or a composition derived therefrom may show a reduction of plant pathogens, including fungal and fungal-like pathogens. A reduction of plant pathogens can be a reduction of about 10% to about 20%, about 20% to about 30%, about 30% to about 40%, about 40% to about 50%, about 50% to about 60%, about 60% to about 70%, about 70% to about 80%, about 80% to about 90%, or about 90% to about 100% when compared to non-treated control plants or plant parts. In other instances, the plant or plant part treated with a bacterial strain provided herein or an active variant thereof, and/or a composition derived therefrom may show a may show a reduction of plant pathogens of at least about 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or about 100% greater when compared to non-treated control plants. Methods for measuring the number of plant pathogens are known, and include contacting plants with one or more pests and determining the plant's ability to survive and/or cause the death of the pests.

By “synergy” or “synergistically” is intended that the combination (applied simultaneously or sequentially) of AIP61892 or an active variant thereof, and a copper compound or AIP1620 or an active variant thereof can (in some embodiments) provide a greater control of a plant pest or plant disease and/or improvement of at least one agronomic trait of interest than the additive effect of AIP61892 and the copper compound or AIP1620 applied individually (i.e., in the absence of the other). This synergy can be an increase of 2%, 5%, 7%, 10%, 12%, 15%, 17%, 20%, 25%, 30%, 35%, 40%, 45%, 50%, 55%, 60%, 65%, 70%, 75%, 80%, 85%, 90%, 100%, or more above the additive effect of the application of AIP61892 and the copper compound or AIP1620 alone (i.e., in the absence of the other). In other embodiments, the application (simultaneous or sequential) of AIP61892 or an active variant thereof, and a copper compound or AIP1620 or an active variant thereof function synergistically when no control of a plant pathogen or treatment or prevention of a plant disease or improvement of at least one agronomic trait of interest is exhibited when AIP61892 or the copper compound or AIP1620 is used alone, but the application (simultaneous or sequential) of the two results in control of a plant pathogen or treatment or prevention of a plant disease or improvement of at least one agronomic trait of interest. In specific embodiments, a syngergistic effect is observed on the control of pests upon the simultaneous or sequential addition of an effective amount of a bacterial strain deposited as NRRL No. B-67089 along with Pseudomonas fluorescens strain AIP1620. For example, the synergisitic effect can be obvserved when an effective amount of a bacterial strain deposited as NRRL No. B-67089 along with Pseudomonas fluorescens strain AIP1620 are administered to a plant or plant part on the control of Rhizoctonia, such as Rhizoctonia solani; Fusarium, such as Fusarium oxysporum; Phytophthora, such as Phytophthora nicotianae; and/or Phythium, such as Pythium ultimum. The synergistic effect can be observed on any plant, and can be observed specifically, on the control of Rhizoctonia, such as Rhizoctonia solani on tomato plants; Fusarium, such as Fusarium oxysporum on tomato plants; Phytophthora, such as Phytophthora nicotianae on tomato plants; and/or Phythium, such as Pythium ultimum on tomato plants.

By “antipathogenic compositions” or “antipathogenic” is intended that the compositions are capable of suppressing, controlling, preventing and/or killing the invading pathogenic organism. In specific embodiments, an antipathogenic composition reduces the disease symptoms resulting from pathogen challenge by a statistically significant amount, including for example, at least about 10% to at least about 20%, at least about 20% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

Assays that measure antipathogenic activity are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, both of which are herein incorporated by reference).

### C. Methods of Inducing Disease and or Pest Resistance in Plants and or for Improving Plant Health and or Improving an Agonomic Trait of Interest

Compositions and methods for inducing disease resistance in a plant to plant pathogens are also provided. Accordingly, the compositions and methods are also useful in protecting plants against fungal pathogens, viruses, nematodes, and insects. Provided herein are methods of inducing disease resistance against a plant pathogen comprising applying to a plant that is susceptible to a plant disease caused by the plant pathogen an effective amount of bacterial strain AIP61892 or an active variant thereof, and/or a composition derived therefrom (and in some embodiments, a copper compound or AIP1620 or variant thereof). In certain embodiments, the bacterial strain provided herein or an active variant thereof, and/or a composition derived therefrom may comprise at least one of AIP61892, or an active variant thereof, or a spore, or a forespore or a combination of cells, forespores and/or spores from any one of AIP61892, or an active variant thereof. In certain embodiments, the bacterial strain provided herein or an active variant thereof, and/or a composition derived therefrom promotes a defensive response to the pathogen that causes the plant disease. In some embodiments, the effective amount of the bacterial strain provided herein or an active variant thereof comprises at least about 10⁴ to 10¹⁶ CFU per hectare. In some embodiments, the effective amount of the bacterial strain provided herein or an active variant thereof comprises at least about 10⁵ to 10¹² CFU per hectare. In some embodiments, the effective amount of the bacterial strain provided herein or an active variant thereof comprises at least about 10¹² to 10¹⁶ CFU per hectare.

A defensive response in the plant can be triggered after applying the bacterial strain provided herein or an active variant thereof, and/or a composition derived therefrom to the plant, but prior to pathogen challenge and/or after pathogen challenge of the plant treated with the bacterial strain provided herein or an active variant thereof, and/or a composition derived therefrom.

In some methods, the bacterial strain provided herein or an active variant thereof, and/or a composition derived therefrom induces resistance to one, two, three, four, five or more plant pathogens described herein. In other methods, the bacterial strain provided herein or an active variant thereof, and/or a composition derived therefrom induces resistance to one, two, three, four, five or more fungal plant pathogens described herein.

By “disease resistance” is intended that the plants avoid the disease symptoms that result from plant-pathogen interactions. That is, pathogens are prevented from causing plant diseases and the associated disease symptoms, or alternatively, the disease symptoms caused by the pathogen are minimized or lessened as compared to a control. By “pest resistance” is intended that the plants avoid the symptoms that result from infection of a plant by a pest. That is, pests are prevented from causing plant diseases and the associated disease symptoms, or alternatively, the disease symptoms caused by the pest are minimized or lessened as compared to a control. Further provided are methods of improving plant health and/or improving an agronomic trait of interest comprising applying to a plant an effective amount of bacterial strain AIP61892 or an active variant thereof, and/or a composition derived therefrom or an active derivative thereof. In certain embodiments, the bacterial strain provided herein or an active variant thereof, and/or a composition derived therefrom may comprise at least one of AIP61892, or an active variant thereof, or a spore, or a forespore or a combination of cells, forespores and/or spores from any one of AIP61892, or an active variant thereof. In some embodiments, the effective amount of the bacterial strain provided herein or an active variant thereof comprises at least about 10⁴ to 10¹⁶ CFU per hectare. In some embodiments, the effective amount of the bacterial strain provided herein or an active variant thereof comprises at least about 10⁵ to 10¹² CFU per hectare. In some embodiments, the effective amount of the bacterial strain provided herein or an active variant thereof comprises at least about 10¹² to 10¹⁶ CFU per hectare. In some embodiments, the composition is derived from a bacteria strain provided herein or an active variant thereof which may comprise a cell of at least one of AIP61892 or an active variant thereof, or a spore, or a forespore or a combination of cells, forespores and/or spores from any one of AIP61892 or an active variant thereof.

In particular embodiments, the agronomic trait of interest that is improved by the bacterial strains or active variants thereof described herein is improved plant health. By “improved plant health” is meant increased growth and/or yield of a plant, increased stress tolerance and/or decreased herbicide resistance, to name a few. Increased stress tolerance refers to an increase in the ability of a plant to decrease or prevent symptoms associated with one or more stresses. The stress can be a biotic stress that occurs as a result of damage done to plants by other living organisms such as a pathogen (for example, bacteria, viruses, fungi, parasites), insects, nematodes, weeds, cultivated or native plants. The stress can also be an abiotic stress such as extreme temperatures (high or low), high winds, drought, salinity, chemical toxicity, oxidative stress, flood, tornadoes, wildfires, radiation and exposure to heavy metals. Non-limiting examples of improved agronomic traits are disclosed elsewhere herein. In specific embodiments, an effective amount of the bacterial strain or an active variant thereof, and/or a composition derived therefrom improves plant health or improves an agronomic trait of interest by a statistically significant amount, including for example, at least about 10% to at least about 20%, at least about 20% to about 50%, at least about 10% to about 60%, at least about 30% to about 70%, at least about 40% to about 80%, or at least about 50% to about 90% or greater.

### D. Methods of Application to a Plant or Plant Part

The bacterial strain provided herein or an active variant thereof, and/or a composition derived therefrom are applied in an effective amount. An effective amount of a bacterial strain provided herein or an active variant thereof, and/or a composition derived therefrom or a combination of AIP61892 or a variant thereof and a copper compound or AIP1620 or a variant thereof is an amount sufficient to control, treat, prevent, inhibit the pathogen or pest that causes a plant disease, and/or reduce plant disease severity or reduce plant disease development. In other embodiments, the effective amount of the bacterial strain provided herein or an active variant thereof, and/or a composition derived therefrom or a combination of AIP61892 or a variant thereof and a copper compound or AIP1620 or a variant thereof is an amount sufficient to improve an agronomic trait of interest and/or to promote or increase plant health, growth or yield of a plant susceptible to a disease and/or infection by a plant pest. The rate of application of the bacterial strain provided herein or an active variant thereof, and/or a composition derived therefrom or a copper compound may vary according to the pathogen or pest being targeted, the crop to be protected, the efficacy of the bacterial strain provided herein or an active variant thereof, and/or a composition derived therefrom, the severity of the disease, the climate conditions, the agronomic trait of interest to improve, and the like.

Generally, the rate of bacterial strain provided herein or an active variant thereof is 10⁴ to 10¹⁶ colony forming units (CFU) per hectare. In other embodiments, for a field inoculation, the rate of bacterial strain provided herein or an active variant thereof application is 3×10⁷ to 1×10¹¹ colony forming units (CFU) per hectare. (This corresponds to about 1 kg to 10 kg of formulated material per hectare). In other embodiments, for a field inoculation, the rate of bacterial strain provided herein or an active variant thereof application is 3×10⁷ to 1×10¹⁶ colony forming units (CFU) per hectare; about 1×10¹² to about 1×10¹³ colony forming units (CFU) per hectare, about 1×10¹³ to about 1×10¹⁴ colony forming units (CFU) per hectare, about 1×10¹⁴ to about 1×10¹⁵ colony forming units (CFU) per hectare, about 1×10¹⁵ to about 1×10¹⁶ colony forming units (CFU) per hectare, about 1×10¹⁶ to about 1×10¹⁷ colony forming units (CFU) per hectare; about 1×10⁴ to about 1×10¹⁶ colony forming units (CFU) per hectare; about 1×10⁵ to about 1×10¹³ colony forming units (CFU) per hectare; about 1×10⁶ to about 1×10¹² colony forming units (CFU) per hectare; about 1×10⁹ to about 1×10¹¹ colony forming units (CFU) per hectare; about 1×10⁹ to about 1×10¹¹ colony forming units (CFU) per hectare. In other embodiments, for a field inoculation, the rate of bacterial strain provided herein or an active variant thereof application is at least about 1×10⁴, about 1×10⁵, about 1×10⁶, about 1×10⁷, about 1×10⁸, about 1×10⁹, about 1×10¹, about 1×10¹¹, about 1×10¹²1×10¹³, about 1×10¹⁴, 1×10¹⁵, about 1×10¹⁶, or about 1×10¹⁷ colony forming units (CFU) per hectare. In some embodiments, for a field inoculation, the rate of bacterial strain provided herein or an active variant thereof application is at least 1×10⁴ to at least about 1×10¹⁶ CFU/hectare. In other embodiments, for a field inoculation, the rate of bacterial strain provided herein or an active variant thereof application is at least 1×10⁷ to at least about 1×10¹⁴ CFU/hectare. In specific embodiments, the bacterial strain provided herein or an active variant thereof applied comprises the strain deposited as AIP61892, or an active derivative of any thereof, or a spore, or a forespore or a combination of cells, forespores and/or spores from any one of AIP61892, or an active derivative of any thereof, or a spore, or a forespore or a combination of cells, forespores and/or spores from any one of AIP61892 or an active derivative of any thereof.

In some embodiments, the applied composition is derived from a bacterial strain or an active variant thereof comprising a strain deposited as AIP61892 or an active derivative of any thereof, or a spore, or a forespore or a combination of cells, forespores and/or spores from any one of AIP61892 or an active derivative of any thereof. In some embodiments, the applied composition may be a substantially pure culture, whole cell broth, supernatant, filtrate, extract, or compound derived from a bacterial strain of the invention or an active variant thereof. The applied composition may be applied alone or in combination with another substance, in an effective amount for controlling a plant pathogen or for improving an agronomic trait of interest in a plant or plant part.

An effective amount of the applied composition is the quantity of microorganism cells, supernatant, whole cell broth, filtrate, cell fraction or extract, metabolite, and/or compound alone or in combination with another pesticidal substance that is sufficient to modulate plant pest infestation or the performance of an agronomic trait of interest in the plant. The amount that will be within an effective range can be determined by laboratory or field tests by one skilled in the art.

In some embodiments wherein an effective amount of a combination of AIP61892 or a variant thereof and a copper compound or AIP1620 or a variant thereof is applied (simultaneously or sequentially), the amount of AIP61892, the AIP1620, or the copper compound is less than the standard amount of the bacterial strains or copper compound when applied alone.

Application (simultaneous or sequential) of AIP61892 or an active variant thereof in combination with a copper compound or AIP1620 or an active variant thereof can occur. In specific embodiments, application of AIP61892 or an active variant thereof is alternated with application of the copper compound or AIP1620 or active variant thereof. Application of AIP61892 or an active variant thereof can alternate with application of a copper compound or AIP1620 or active variant thereof, by 0.5 days, 1 day, 1.5 days, 2 days, 3 days, 4 days, 5 days, 6 days, 7 days, 10 days, 14 days or more. In some embodiments, AIP61892 or active varian thereof is applied to a plant, plant part, or cultivation area of a plant, simultaneously with a copper compound or AIP1620 or active variant thereof. Simultaneous application of AIP61892 or active variant thereof and a copper compound or AIP1620 or active variant thereof can occur in the same formulation or simultaneously from separate formulations.

Sequential application of AIP61892 or active variant thereof and a copper compound or AIP1620 or active variant thereof refers to the application of each of the components that does not occur simultaneously. Sequential application of two components includes the application of a component a) before or after a component b), wherein the difference in time between application of components a) and b) is greater than 1 minute, including but not limited to 1 minute, 5 minutes, 30 minutes, 1 hour, 2 hours, 5 hours, 10 hours, 1 day, 2 days, 3 days, 4 days, 5 days, 6 days, 7 days, 8 days, 9 days, 10 days, 15 days, 20 days, 1 month, 2 months, 3 months, 4 months, 5 months, 6 months, 7 months, 8 months, 9 months, 10 months, 11 months, 1 year or greater, wherein the timing of the sequential application of the two components is such that an additive effect or synergistic effect (on the controlling of a plant pathogen or treatment or prevention of a plant disease, or an improvement of an agronomic trait) is observed.

In some embodiments, when the composition is applied directly to the seed, the effective amount is a concentration of about 0.05-25%, or about 0.1-20%, or about 0.5-15%, or about 1-10%, or about 2-5% of the active ingredient per 100 g of seed. In some embodiments, the effective amount is about 0.5-1% of the active ingredient per 100 g of seed.

In some embodiments, when the composition is applied to the soil by, for example, in furrow, the effective amount is about 0.1-50 oz. of the active ingredient per 1000 ft row. In another embodiment, the effective amount for soil application is about 1-25 oz. of the active ingredient per 1000 ft row. In another embodiment, the effective amont is about 2-20 oz, or about 3-15 oz, or about 4-10 oz, or about 5-8 oz, of the active ingredient per 1000 ft row. In yet another embodiment, the effective amount is about 14 or 28 oz of the active ingredient per 1000 ft row.

Any appropriate agricultural application rate for a biocide can be applied in combination with the bacterial strain provided herein or an active variant thereof, and/or a composition derived therefrom disclosed herein. Methods to assay for the effective amount of the bacterial strain provided herein or an active variant thereof, and/or a composition derived therefrom include, for example, any statistically significant increase in the control of the pathogen or pest targeted by the biocide. Methods to assay for such control are known. Moreover, a statistically significant increase in the control of plant health, yield and/or growth that occurs upon application of an effective amount of the bacterial strain provided herein or an active variant thereof, and/or a composition derived therefrom when compared to the plant health, yield and/or growth that occurs when no bacterial strain provided herein or an active variant thereof, and/or a composition derived therefrom is applied.

In some embodiments, bacterial strain AIP1620 is applied in combination with the bacterial strain AIP61892 or an active variant thereof, and/or a composition derived therefrom disclosed herein. An effective amount of bacterial strain AIP1620 comprises at least about 10⁵ to 10¹² CFU per hectare, or at least about 10⁷ to 10¹⁴ total cells per hectare.

Further provided is a method for controlling or inhibiting the growth of a plant pest or a plant pathogen that causes plant disease by applying a composition comprising bacterial strain AIP61892 or an active variant thereof, and/or a composition derived therefrom provided herein (i.e., AIP61892, or an active variant or any thereof, or a spore, or a forespore or a combination of cells, forespores and/or spores from AIP61892, or an active variant any of thereof). By “applying” is intended contacting an effective amount of the bacterial strain provided herein or an active variant thereof, and/or a composition derived therefrom to a plant, area of cultivation, seed and/or weed with one or more of the bacterial strain provided herein or an active variant thereof, and/or a composition derived therefrom so that a desired effect is achieved. Furthermore, the application of the bacterial strain provided herein or an active variant thereof, and/or a composition derived therefrom can occur prior to the planting of the crop (for example, to the soil, the seed, or the plant). In a specific embodiment, the application of the bacterial strain provided herein or an active variant thereof, and/or a composition derived therefrom is a foliar application. Therefore, a further embodiment of the invention provides a method for controlling or inhibiting the growth of a plant pest by applying the population of bacterial strain provided herein or an active variant thereof, and/or a composition derived therefrom to an environment in which the plant pest may grow. The application may be to the plant, to parts of the plant, to the seeds of the plants to be protected, or to the soil in which the plant to be protected are growing or will grow. Application to the plant or plant parts may be before or after harvest. Application to the seeds will be prior to planting of the seeds.

In some embodiments, an effective amount of bacterial strain AIP61892 or an active variant thereof provided herein is used as a foliar application to control or inhibit growth of one or more pathogens selected from the group consisting of Alternaria spp., Alternaria solani, Colletotrichum spp., Erysiphe spp., Mycosphaerella spp., Phomopsis spp., Podosphaera spp., Cercospora spp., Botrytis spp., Uncinula spp., Erwinia spp., Pseudomonas spp., and Xanthomonas spp.

In other embodiments, an effective amount of bacterial strain AIP61892 or an active variant thereof, and/or a composition derived therefrom provided herein is applied to the soil in which the plant to be protected are growing or will grow to control or inhibit growth of one or more pathogens selected from the group consisting of Rhizoctonia spp., Rhizoctonia solani, Fusarium spp., Sclerotium spp., Sclerotinia spp., Sclerotinia sclerotiorum, Phytopthora spp., and Pythium spp.

In some embodiments, an effective amount of bacterial strain AIP61892 or an active variant thereof, and/or a composition derived therefrom provided herein is applied to the plant after harvest to control or inhibit growth of one or more pathogens.

In some embodiments, the plant or plant part has been harvested or otherwise removed from the field or area of cultivation. Methods are provided for controlling a plant pest after harvest, also referred to as post-harvest. In some embodiments, the plant pest is a plant pathogen. In further embodiments, the plant pathogen is a mycotoxin-producing fungus. Mycotoxins are known to be produced by Aspergillus spp., Penicillium spp., Fusarium spp., and Claviceps spp. (Liu et al. (2020) Comprehensive Reviews in Food Science and Food Safety 19:1521-1560, incorporated by reference herein). Post-harvest plant pathogens include mycotoxin-producing fungi and rotting fungal and bacterial pathogens, including Aspergillus spp., Botrytis spp., Fusarium spp., Phacidiopynis spp., Sphaeropsis spp., Botryosphaeria spp., Rhizopus spp., Claviceps spp., Colletotrichum spp., Geotricum spp., Diaporthe spp., Mucor spp., Lasiodiplodis spp., Neofabrea spp., Monilinia spp., Gilbertella spp., Penicillium spp., Erwinia spp., Pectobacterium spp., and Brenneria spp.

An effective amount of a composition comprising bacterial strain AIP61892 or an active variant thereof can be applied to a plant or plant part prior to harvesting or after the plant or plant part has been harvested. As used herein, the term harvesting refers to the removal of a plant or plant part from the ground or other area of cultivation and can also refer to removal of a plant part from a plant that remains in the ground or other area of cultivation. The plant part may be a fruit, which may be harvested from a tree, bush, or vine, or it may be the edible portion of a vegetable crop such as a leaf, tuber, or root. Examples of plant parts include pome fruit, citrus, stone fruit, berries, tomatoes, peppers, melons, and tropical fruit. In some embodiments, the plant part is the grain or seed of the plant. Methods of post-harvest application include coating, spraying (high volume or low volume), fogging, thermofogging, drenching, dipping, flooding, foaming, brushing, or dusting the harvested plant or plant part with a composition comprising a bacterial strain described herein or a variant thereof. Other methods of post-harvest application include using paper wraps or box liners that have been treated with a composition comprising or derived from a bacterial strain described herein or a variant thereof. The composition comprising or derived from a bacterial strain described herein or a variant thereof may be aqueous or a wax-oil emulsion. In some embodiments, the composition is a fruit coating further comprising non-emulsified mineral oil, emulsified mineral oil, polyethylene, vegetable oil, carnauba, shellac, a wood rosin blend, or a combination of any these.

In certain embodiments, the bacterial strain provided herein or an active variant thereof, and/or a composition derived therefrom may comprise at least one of AIP61892 or an active variant thereof; or a spore, or a forespore or a combination of cells, forespores and/or spores from any one of AIP61892 or an active variant thereof. Various effective amounts of bacterial strain provided herein or an active variant thereof are disclosed elsewhere herein and in one, non-limiting example, the effective amount of the bacterial strain provided herein or an active variant thereof comprises at least about 10⁴ to 10¹⁶ CFU/g, about 10¹² to 10¹⁶ CFU/g, about 10⁴ to 10¹² CFU/mL, about 10¹² to 10¹⁶ CFU/mL, or equivalent measure of bacterial concentration. In some embodiments, the composition further comprises at least one synthetic pesticide, such as for example imazalil, pyrimethanil, fludioxonil, azoxystrobin, propiconazole, tebucanozole, difenoconazole, or any synthetic pesticide recited elsewhere herein. In high volume applications, for example by a T-jet, a bacterial strain of the invention or a variant thereof may be supplied at about 10⁴ to 10¹⁶ CFU/mL at 100-200 gallons/ton of fruit. In low volume applications, for example by controlled droplet application, a bacterial strain of the invention or a variant thereof may be supplied at about 10⁴ to 10¹⁶ CFU/mL at 8-30 gallons/ton of fruit.

In specific embodiments, the application of the bacterial strain provided herein or an active variant thereof, and/or a composition comprising the same or derived therefrom (i.e., AIP61892, or an active variant thereof, or a spore, or a forespore or a combination of cells, forespores and/or spores from any one of AIP61892, or an active variant thereof, and/or a composition derived therefrom) is applied to the leaves of a soybean plant. The timing of application can vary depending on the conditions and geographical location. In specific embodiments, the bacterial strain provided herein or an active variant thereof, and/or a composition derived therefrom is applied at the R1 (beginning flowering stage) of soybean development or may be applied earlier depending on disease onset and the disease severity.

In other embodiments, the biocide to a crop, area of cultivation, or field it is intended that one or more of a particular field, plant crop, seed and/or weed is treated with one or more of the bacterial strain provided herein or an active variant thereof, and/or a composition derived therefrom and one or more biocide so that a desired effect is achieved.

Various methods are provided for controlling a plant pest that causes a plant disease in an area of cultivation containing a plant susceptible to the plant disease. The method comprises planting the area of cultivation with seeds or plants susceptible to the plant disease or pest; and applying to the plant susceptible to the disease or pest, the seed or the area of cultivation of the plant susceptible to the plant disease or pest an effective amount of bacterial strain AIP61892 or an active variant thereof, and/or a composition comprising the same or derived therefrom (i.e., AIP61892, or an active derivative or any thereof, or a spore, or a forespore or a combination of cells, forespores and/or spores from AIP61892, or an active variant thereof), wherein the effective amount of the bacterial strain provided herein or an active variant thereof, and/or a composition derived therefrom controls the plant disease without significantly affecting the crop. In some embodiments, the effective amount of the bacterial strain provided herein or an active variant thereof comprises at least about 10⁴ to 10¹⁶ CFU per hectare. In some embodiments, the effective amount comprises at least about 10⁵ to 10¹² colony forming units (CFU) per hectare. In other embodiments, the effective amount comprises at least about 10¹² to 10¹⁶ colony forming units (CFU) per hectare. In some embodiments, the composition is derived from a bacterial strain provided herein or an active variant thereof and may comprise a cell of at least one of AIP61892 or an active variant thereof, or a spore, or a forespore or a combination of cells, forespores and/or spores from any one of AIP61892 or an active variant thereof.

Further provided is a method for growing a plant susceptible to a plant disease or plant pest. The method comprises applying to a plant susceptible to the disease or pest, a seed, or an area of cultivation of the plant susceptible to the disease or pest an effective amount of a composition comprising bacterial strain AIP61892 or an active variant thereof, and/or a composition comprising the same or derived therefrom. In certain embodiments, the bacterial strain provided herein or an active variant thereof, and/or a composition derived therefrom may comprise AIP61892, or an active variant thereof, or a spore, or a forespore or a combination of cells, forespores and/or spores of AIP61892, or an active variant thereof. Various effective amounts of a bacterial strain provided herein or an active variant thereof are disclosed elsewhere herein and in one, non-limiting example, the effective amount of the bacterial strain provided herein or an active variant thereof comprises at least about 10⁴ to 10¹⁶ colony forming units (CFU) per hectare, at least about 10⁵ to 10¹² colony forming units (CFU) per hectare, or at least about 10¹² to 10¹⁶ colony forming units (CFU) per hectare.

Methods are provided for controlling a plant pest on a plant or plant part by applying to the plant or plant part an effective amount of a composition comprising bacterial strain AIP61892 or an active variant thereof, and/or a composition comprising the same or derived therefrom. Methods are also provided for controlling a plant pest by contacting said pest with an effective amount of a composition comprising bacterial strain AIP61892 or an active variant thereof, and/or a composition comprising the same or derived therefrom. Various effective amounts of bacterial strain provided herein or an active variant thereof are disclosed elsewhere herein. In one, non-limiting example, the effective amount of a bacterial strain provided herein or an active variant thereof comprises at least about 10⁴ to 10¹⁶ colony forming units (CFU) per hectare, at least about 10⁴ to 10¹² colony forming units (CFU) per hectare, or at least about 10¹² to 10¹⁶ colony forming units (CFU) per hectare.

The composition comprising the bacterial strain or an active variant thereof can be a solid or liquid composition or formulation. The plant or plant part need not be actively growing in order for the bacterial strain to effectively control the plant pest.

Methods for increasing plant yield are provided. The “yield” of the plant refers to the quality and/or quantity of biomass produced by the plant. By “biomass” is intended any measured plant product. An increase in biomass production is any improvement in the yield of the measured plant product. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield compared to a plant not exposed to the bacterial strain provided herein or an active variant thereof, and/or a composition derived therefrom. A method for increasing yield in a plant is also provided and comprises applying to a crop or an area of cultivation an effective amount of a composition comprising at least one bacterial strain comprising AIP61892 or an active variant thereof, a spore or a forespore or a combination of cells, forespores and/or spores from any one of AIP61892 or an active variant thereof, wherein said effective amount comprises at least about 10⁴ to 10¹⁶ colony forming units (CFU) per hectare, at least about 10⁵ to 10¹² colony forming units (CFU) per hectare, or at least about 10¹² to 10¹⁶ colony forming units (CFU) per hectare, and wherein said composition controls a plant pathogen or other plant pest, thereby increasing yield. A method for increasing yield in a plant is also provided which comprises applying to a crop or an area of cultivation an effective amount of a composition derived from at least one bacterial strain comprising AIP61892 or an active variant thereof, a spore or a forespore or a combination of cells, forespores and/or spores from any one of AIP61892 or an active variant thereof, wherein said composition controls a plant pest, thereby increasing yield.

As used herein, an “area of cultivation” comprises any region in which one desires to grow a plant. Such areas of cultivations include, but are not limited to, a field in which a plant is cultivated (such as a crop field, a sod field, a tree field, a managed forest, a field for culturing fruits and vegetables, etc.), a greenhouse, a growth chamber, etc.

Further provided is a coated seed which comprises a seed and a coating on the seed, wherein the coating comprises bacterial strain AIP61892 or an active variant thereof, and/or a composition comprising the same or derived therefrom. In certain embodiments, the bacterial strain provided herein or an active variant thereof, and/or a composition derived therefrom may comprise at least one of AIP61892 or an active variant thereof, or a spore, or a forespore or a combination of cells, forespores and/or spores from any one of AIP61892 or an active variant thereof. In certain embodiments, said bacterial strain provided herein or an active variant thereof is present on the seed at about 10⁵ CFU/100 lbs of seed to about 10⁷ CFU/100 lbs of seed, at about 10⁴ CFU/100 lbs of seed to about 10⁸ CFU/100 lbs of seed, at about 10⁴ CFU/100 lbs of seed to about 10⁵ CFU/100 lbs of seed, at about 10⁵ CFU/100 lbs of seed to about 10⁶ CFU/100 lbs of seed, at about 10⁶ CFU/100 lbs of seed to about 10⁷ CFU/100 lbs of seed, or at about 10⁷ CFU/100 lbs of seed to about 10⁸ CFU/100 lbs of seed. The seed coating can be applied to any seed of interest (i.e., for a monocotyledonous plant or a dicotyledonous plant). Various plants of interest are disclosed elsewhere herein.

The seed coating may improve the health of the seed prior to being placed into a germination media, for example soil. In some embodiments, the seed coating may improve the health of the germinating seedling compared to a germinating seedling from a seed that does not have a seed coating. The seed coating may control plant pests, such as nematodes (for example Meloidogyne spp., Globodera spp., and Heterodera spp.), fungal, fungal-like, or bacterial pathogens, that infect seed or germinating seedlings. The seed coating may control pathogens that cause diseases such as damping off, vascular wilts, or rot. Such pathogens include Fusarium spp., Pythium spp., Rhizoctonia spp., Phytophthora spp., and Verticillium spp.

A seed coating can further comprise at least at least one nutrient, at least one herbicide or at least one pesticide, or at least one biocide. See, for example, US App Pub. 20040336049, 20140173979, and 20150033811. In some embodiments, the seed coating further comprises a pesticide, fungicide, nematicide, bactericide, insecticide, or an herbicide, such as those recited elsewhere herein. In some embodiments, the seed coating may further comprise bacterial strain AIP1620. AIP1620 may also be present on the seed at 10⁵ CFU/100 lbs of seed to about 10⁷ CFU/100 lbs of seed, at about 10⁴ CFU/100 lbs of seed to about 10⁸ CFU/100 lbs of seed, at about 10⁴ CFU/100 lbs of seed to about 10⁵ CFU/100 lbs of seed, at about 10⁵ CFU/100 lbs of seed to about 10⁶ CFU/100 lbs of seed, at about 10⁶ CFU/100 lbs of seed to about 10⁷ CFU/100 lbs of seed, or at about 10⁷ CFU/100 lbs of seed to about 10⁸ CFU/100 lbs of seed. In some embodiments, the seed coating may further comprise bacterial strain AIP1620 at about 10⁷ total cells/100 lbs of seed to about 10¹² total cells/100 lbs of seed, at about 10⁸ total cells/100 lbs of seed to about 10¹³ total cells/100 lbs of seed, at about 10¹⁰ total cells/100 lbs of seed to about 10¹¹ total cells/100 lbs of seed, at about 10⁷ total cells/100 lbs of seed to about 10¹⁴ total cells/100 lbs of seed, at about 10⁸ total cells/100 lbs of seed to about 10¹⁰ total cells/100 lbs of seed, at about 10⁹ total cells/100 lbs of seed to about 10¹³ total cells/100 lbs of seed, at about 10⁸ total cells/100 lbs of seed to about 10¹³ total cells/100 lbs of seed, at about 10⁸ total cells/100 lbs of seed to about 10¹⁴ total cells/100 lbs of seed, or at about 10⁷ total cells/100 lbs of seed to about 10¹⁴ total cells/100 lbs of seed.

A plant or plant part is provided having at least one bacterial strain disclosed herein or an active variant thereof, and/or a composition derived therefrom applied to the surface of the plant or plant part. The bacterial strain, or an active variant thereof applied to the surface of the plant or plant part can be in the form a composition or formulation as disclosed elsewhere herein. In specific embodiments the bacterial strain provided herein or an active variant thereof is applied to a plant that has been removed from the field or area of cultivation or applied to a plant part that has been removed from the plant. In certain embodiments, the bacterial strain provided herein or an active variant thereof, and/or a composition derived therefrom may comprise at least one of AIP61892 or an active variant thereof, or a spore, or a forespore or a combination of cells, forespores and/or spores from any one of AIP61892 or an active variant thereof. In certain embodiments, said bacterial strain provided herein or an active variant thereof is applied to the plant or plant part at a concentration of about 10⁴ to 10¹² CFU/g, about 10¹² to 10¹⁶ CFU/g, about 10⁴ to 10¹² CFU/mL, about 10¹² to 10¹⁶ CFU/mL, or equivalent measure of bacterial concentration.

In other embodiments, a plant of interest (i.e., plant susceptible to the plant disease), a plant part of interest, and/or the area of cultivation comprising the plant, can be treated with a combination of an effective amount of the bacterial strain provided herein or an active variant thereof, and/or a composition derived therefrom and an effective amount of a biocide. By “treated with a combination of” or “applying a combination of” a bacterial strain provided herein or an active variant thereof, and/or a composition derived therefrom and a biocide to a plant, plant part, area of cultivation or field it is intended that one or more of a particular field, plant, plant part, and/or weed is treated with an effective amount of one or more of the bacterial strain provided herein or an active variant thereof, and/or a composition derived therefrom and one or more biocide so that a desired effect is achieved. Furthermore, the application of one or both of the bacterial strain provided herein or an active variant thereof, and/or a composition derived therefrom and the biocide can occur prior to the planting of the crop (for example, to the soil, or the plant) and/or after harvesting the crop. Moreover, the application of the bacterial strain provided herein or an active variant thereof, and/or a composition derived therefrom and the biocide may be simultaneous or the applications may be at different times (sequential), so long as the desired effect is achieved.

In one non-limiting embodiment, the active variant comprises a bacterial strain provided herein that is resistance to one or more biocide. In specific embodiments, the bacterial strain provided herein or an active variant thereof, and/or a composition derived therefrom (i.e., AIP61892 or an active variant thereof, or a spore, or a forespore or a combination of cells, forespores and/or spores from AIP61892, or an active variant thereof) is resistant to glyphosate. In such methods, a plant, crop, or area of cultivation is treated with a combination of an effective amount of the bacterial strain provided herein or an active variant thereof, and/or a composition derived therefrom that is resistant to glyphosate and an effective amount of glyphosate, wherein the effective amount of glyphosate is such as to selectively control weeds while the crop is not significantly damaged.

In another non-limiting embodiment, the active variant comprises a bacterial strain provided herein that is resistant to glufosinate. In such methods, a plant, crop, or area of cultivation is treated with a combination of an effective amount of the bacterial strain provided herein or an active variant thereof, and/or a composition derived therefrom that is resistant to glufosinate and an effective amount of glufosinate, wherein the effective amount of glufosinate is such as to selectively control weeds while the crop is not significantly damaged. In such embodiments, the effective amount of the bacterial strain provided herein or an active variant thereof, and/or a composition derived therefrom is sufficient to result in a statistically significant increase in plant health, yield, and/or growth when compared to the plant health, yield, and/or growth that occurs when the same concentration of a bacterial strain provided herein or an active variant thereof, and/or a composition derived therefrom that was not modified to be resistant to glufosinate is applied in combination with the effective amount of the glufosinate or active derivative thereof. In a further embodiment, a bacterial strain provided herein or active variant therof, and/or a composition derived therefrom comprises an effective amount of AIP61892 or an active variant thereof, or a spore, or a forespore or a combination of cells, forespores and/or spores from any one of AIP61892 or an active variant thereof.

### V. Biocides for Use in Combination with the Bacterial Strains Provided Herein or an Active Variant Thereof and or a Composition Derived Therefrom

As discussed elsewhere herein, the bacterial strain provided herein or active variant therof, and/or a composition derived therefrom can be used in combination with a biocide (i.e., an herbicide, fungicide, pesticide, or other crop protection chemical). In such instances, the bacterial strain provided herein or an active variant thereof, and/or a composition derived therefrom is compatible with the biocide of interest. In some embodiments, the bacterial strain provided herein or an active variant thereof, and/or a composition derived therefrom is provided as a stable formulation which further comprises a herbicide, fungicide, bactericide, nematicide, pesticide, insecticide or other crop protection chemical. In some embodiments, the bacterial strain provided herein or an active variant thereof, and/or a composition derived therefrom is combined in a tank mix which further comprises a herbicide, fungicide, bactericide, nematicide, pesticide, insecticide or other crop protection chemical.

Herbicides that can be used in the various methods and compositions discloses herein include glyphosate, ACCase inhibitors (Arloxyphenoxy propionate (FOPS)); ALS inhibitors (Sulfonylurea (SU)), Imidazonlinone (IMI), Pyrimidines (PM)); microtubule protein inhibitor (Dinitroaniline (DNA)); synthetic auxins (Phenoxy (P)), Benzoic Acid (BA), Carboxylic acid (CA)); Photosystem II inhibitor (Triazine (TZ)), Triazinone (TN), Nitriles (NT), Benzothiadiazinones (BZ), Ureas (US)); EPSP Synthase inhibitor (glycines (GC)); Glutamine Synthesis inhibitor (Phosphinic Acid (PA)); DOXP synthase inhibitor (Isoxazolidinone (IA)); HPPD inhibitor (Pyrazole (PA)), Triketone (TE)); PPO inhibitors (Diphenylether (DE), N-phenylphthalimide (NP) (Ary triazinone (AT)); VLFA inhibitors (chloroacetamide (CA)), Oxyacetamide (OA)); Photosystem I inhibitor (Bipyridyliums (BP)); and the like.

Pesticides that can be used in the various methods and compositions disclosed herein include imidacloprid clothianidin, arylpyrazole compounds (WO2007103076); organophosphates, phenyl pyrazole, pyrethoids caramoyloximes, pyrazoles, amidines, halogenated hydrocarbons, carbamates and derivatives thereof, terbufos, chloropyrifos, fipronil, chlorethoxyfos, telfuthrin, carbofuran, imidacloprid, tebupirimfos (U.S. Pat. No. 5,849,320).

Nematicides that can be used in the various methods and compositions disclosed herein include any synthetic nematicide or biological nematicide. Examples of synthetic nematicides include, but are not limited to, acibenzolar-S-methyl, an avermectin (e.g., abamectin), carbamate nematicides (e.g., aldicarb, thiadicarb, carbofuran, carbosulfan, oxamyl, aldoxycarb, ethoprop, methomyl, benomyl, alanycarb), organophosphorus nematicides (e.g., phenamiphos (fenamiphos), fensulfothion, terbufos, fosthiazate, dimethoate, phosphocarb, dichlofenthion, isamidofos, fosthietan, isazofos ethoprophos, cadusafos, terbufos, chlorpyrifos, dichlofenthion, heterophos, isamidofos, mecarphon, phorate, thionazin, triazophos, diamidafos, fosthietan, phosphamidon), and certain fungicides, such as captan, thiophanate-methyl and thiabendazole. Biological nematicides include, but are not limited to, nematicides include ARF18; Arthrobotrys spp.; Chaetomium spp.; Cylindrocarpon spp.; Exophilia spp.; Fusarium spp.; Gliocladium spp.; Hirsutella spp.; Lecanicillium spp.; Monacrosporium spp.; Myrothecium spp.; Neocosmospora spp.; Paecilomyces spp.; Pochonia spp.; Stagonospora spp.; vesicular-arbuscular mycorrhizal fungi, Burkholderia spp.; Pasteuria spp., Brevibacillus spp.; Pseudomonas spp.; Rhizobacteria; and Bacillus spp.

Fungicides and/or bactericides that can be used in the various methods and compositions disclosed herein include aliphatic nitrogen fungicides (butylamine, cymoxanil, dodicin, dodine, guazatine, iminoctadine); amide fungicides (benzovindiflupyr, carpropamid, chloraniformethan, cyflufenamid, diclocymet, diclocymet, dimoxystrobin, fenaminstrobin, fenoxanil, flumetover, furametpyr, isofetamid, isopyrazam, mandestrobin, mandipropamid, metominostrobin, orysastrobin, penthiopyrad, prochloraz, quinazamid, silthiofam, triforine); acylamino acid fungicides (benalaxyl, benalaxyl-M, furalaxyl, metalaxyl, metalaxyl-M, pefurazoate, valifenalate); anilide fungicides (benalaxyl, benalaxyl-M, bixafen, boscalid, carboxin, fenhexamid, fluxapyroxad, isotianil, metalaxyl, metalaxyl-M, metsulfovax, ofurace, oxadixyl, oxycarboxin, penflufen, pyracarbolid, sedaxane, thifluzamide, tiadinil, vanguard); benzanilide fungicides (benodanil, flutolanil, mebenil, mepronil, salicylanilide, tecloftalam); furanilide fungicides (fenfuram, furalaxyl, furcarbanil, methfuroxam); sulfonanilide fungicides (flusulfamide); benzamide fungicides (benzohydroxamic acid, fluopicolide, fluopyram, tioxymid, trichlamide, zarilamid, zoxamide); furamide fungicides (cyclafuramid, furmecyclox); phenylsulfamide fungicides (dichlofluanid, tolylfluanid); sulfonamide fungicides (amisulbrom, cyazofamid); valinamide fungicides (benthiavalicarb, iprovalicarb); antibiotic fungicides (aureofungin, blasticidin-S, cycloheximide, griseofulvin, kasugamycin, moroxydine, natamycin, polyoxins, polyoxorim, streptomycin, validamycin); strobilurin fungicides (fluoxastrobin, mandestrobin); methoxyacrylate strobilurin fungicides (azoxystrobin, bifujunzhi, coumoxystrobin, enoxastrobin, flufenoxystrobin, jiaxiangjunzhi, picoxystrobin, pyraoxystrobin); methoxycarbanilate strobilurin fungicides (pyraclostrobin, pyrametostrobin, triclopyricarb); methoxyiminoacetamide strobilurin fungicides (dimoxystrobin, fenaminstrobin, metominostrobin, orysastrobin); methoxyiminoacetate strobilurin fungicides (kresoxim-methyl, trifloxystrobin); aromatic fungicides (biphenyl, chlorodinitronaphthalenes, chloroneb, chlorothalonil, cresol, dicloran, fenjuntong, hexachlorobenzene, pentachlorophenol, quintozene, sodium pentachlorophenoxide, tecnazene, trichlorotrinitrobenzenes); arsenical fungicides (asomate, urbacide); aryl phenyl ketone fungicides (metrafenone, pyriofenone); benzimidazole fungicides (albendazole, benomyl, carbendazim, chlorfenazole, cypendazole, debacarb, fuberidazole, mecarbinzid, rabenzazole, thiabendazole); benzimidazole precursor fungicides (furophanate, thiophanate, thiophanate-methyl); benzothiazole fungicides (bentaluron, benthiavalicarb, benthiazole, chlobenthiazone, probenazole); botanical fungicides (allicin, berberine, carvacrol, carvone, osthol, sanguinarine, santonin); bridged diphenyl fungicides (bithionol, dichlorophen, diphenylamine, hexachlorophene, parinol); carbamate fungicides (benthiavalicarb, furophanate, iodocarb, iprovalicarb, picarbutrazox, propamocarb, pyribencarb, thiophanate, thiophanate-methyl, tolprocarb); benzimidazolylcarbamate fungicides (albendazole, benomyl, carbendazim, cypendazole, debacarb, mecarbinzid); carbanilate fungicides (diethofencarb, pyraclostrobin, pyrametostrobin, triclopyricarb); conazole fungicides, conazole fungicides (imidazoles) (climbazole, clotrimazole, imazalil, oxpoconazole, prochloraz, triflumizole); conazole fungicides (triazoles) (azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P); copper fungicides (acypetacs-copper, Bordeaux mixture, Burgundy mixture, Cheshunt mixture, copper acetate, basic copper carbonate, copper hydroxide, copper linoleate, copper naphthenate, a copper compound such as copper oleate, copper oxychloride, copper octanoate, copper silicate, copper sulfate, copper sulfate pentahydrate, tribasic copper sulfate, or copper zinc chromate; cufraneb, cuprobam, cuprous oxide, mancopper, oxine-copper, saisentong, thiodiazole-copper); cyanoacrylate fungicides (benzamacril, phenamacril); dicarboximide fungicides (famoxadone, fluoroimide); dichlorophenyl dicarboximide fungicides (chlozolinate, dichlozoline, iprodione, isovaledione, myclozolin, procymidone, vinclozolin); phthalimide fungicides (captafol, captan, ditalimfos, folpet, thiochlorfenphim); dinitrophenol fungicides (binapacryl, dinobuton, dinocap, dinocap-4, dinocap-6, meptyldinocap, dinocton, dinopenton, dinosulfon, dinoterbon, DNOC); dithiocarbamate fungicides (amobam, asomate, azithiram, carbamorph, cufraneb, cuprobam, disulfiram, ferbam, metam, nabam, tecoram, thiram, urbacide, ziram); cyclic dithiocarbamate fungicides (dazomet, etem, milneb); polymeric dithiocarbamate fungicides (mancopper, mancozeb, maneb, metiram, polycarbamate, propineb, zineb); dithiolane fungicides (isoprothiolane, saijunmao); fumigant fungicides (carbon disulfide, cyanogen, dithioether, methyl bromide, methyl iodide, sodium tetrathiocarbonate); hydrazide fungicides (benquinox, saijunmao); imidazole fungicides (cyazofamid, fenamidone, fenapanil, glyodin, iprodione, isovaledione, pefurazoate, triazoxide); conazole fungicides (imidazoles) (climbazole, clotrimazole, imazalil, oxpoconazole, prochloraz, triflumizole); inorganic fungicides (potassium azide, potassium thiocyanate, sodium azide, sulfur, see also copper fungicides, see also inorganic mercury fungicides); mercury fungicides; inorganic mercury fungicides (mercuric chloride, mercuric oxide, mercurous chloride); organomercury fungicides ((3-ethoxypropyl)mercury bromide, ethylmercury acetate, ethylmercury bromide, ethylmercury chloride, ethylmercury 2,3-dihydroxypropyl mercaptide, ethylmercury phosphate, N-(ethylmercury)-p-toluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivative of pyrocatechol, phenylmercury nitrate, phenylmercury salicylate, thiomersal, tolylmercury acetate); morpholine fungicides (aldimorph, benzamorf, carbamorph, dimethomorph, dodemorph, fenpropimorph, flumorph, tridemorph); organophosphorus fungicides (ampropylfos, ditalimfos, EBP, edifenphos, fosetyl, hexylthiofos, inezin, iprobenfos, izopamfos, kejunlin, phosdiphen, pyrazophos, tolclofos-methyl, triamiphos); organotin fungicides (decafentin, fentin, tributyltin oxide); oxathiin fungicides (carboxin, oxycarboxin); oxazole fungicides (chlozolinate, dichlozoline, drazoxolon, famoxadone, hymexazol, metazoxolon, myclozolin, oxadixyl, oxathiapiprolin, pyrisoxazole, vinclozolin); polysulfide fungicides (barium polysulfide, calcium polysulfide, potassium polysulfide, sodium polysulfide); pyrazole fungicides (benzovindiflupyr, bixafen, fenpyrazamine, fluxapyroxad, furametpyr, isopyrazam, oxathiapiprolin, penflufen, penthiopyrad, pyraclostrobin, pyrametostrobin, pyraoxystrobin, rabenzazole, sedaxane); pyridine fungicides (boscalid, buthiobate, dipyrithione, fluazinam, fluopicolide, fluopyram, parinol, picarbutrazox, pyribencarb, pyridinitril, pyrifenox, pyrisoxazole, pyroxychlor, pyroxyfur, triclopyricarb); pyrimidine fungicides (bupirimate, diflumetorim, dimethirimol, ethirimol, fenarimol, ferimzone, nuarimol, triarimol); anilinopyrimidine fungicides (cyprodinil, mepanipyrim, pyrimethanil); pyrrole fungicides (dimetachlone, fenpiclonil, fludioxonil, fluoroimide); quaternary ammonium fungicides (berberine, sanguinarine); quinoline fungicides (ethoxyquin, halacrinate, 8-hydroxyquinoline sulfate, quinacetol, quinoxyfen, tebufloquin); quinone fungicides (chloranil, dichlone, dithianon); quinoxaline fungicides (chinomethionat, chlorquinox, thioquinox); thiadiazole fungicides (etridiazole, saisentong, thiodiazole-copper, zinc thiazole); thiazole fungicides (ethaboxam, isotianil, metsulfovax, octhilinone, oxathiapiprolin, thiabendazole, thifluzamide); thiazolidine fungicides (flutianil, thiadifluor); thiocarbamate fungicides (methasulfocarb, prothiocarb); thiophene fungicides (ethaboxam, isofetamid, silthiofam); triazine fungicides (anilazine); triazole fungicides (amisulbrom, bitertanol, fluotrimazole, triazbutil); conazole fungicides (triazoles) (azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, huanjunzuo, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P); triazolopyrimidine fungicides (ametoctradin); urea fungicides (bentaluron, pencycuron, quinazamid); zinc fungicides (acypetacs-zinc, copper zinc chromate, cufraneb, mancozeb, metiram, polycarbamate, polyoxorim-zinc, propineb, zinc naphthenate, zinc thiazole, zinc trichlorophenoxide, zineb, ziram); unclassified fungicides (acibenzolar, acypetacs, allyl alcohol, benzalkonium chloride, bethoxazin, bromothalonil, chitosan, chloropicrin, DBCP, dehydroacetic acid, diclomezine, diethyl pyrocarbonate, ethylicin, fenaminosulf, fenitropan, fenpropidin, formaldehyde, furfural, hexachlorobutadiene, methyl isothiocyanate, nitrostyrene, nitrothal-isopropyl, OCH, pentachlorophenyl laurate, 2-phenylphenol, phthalide, piperalin, propamidine, proquinazid, pyroquilon, sodium orthophenylphenoxide, spiroxamine, sultropen, thicyofen, tricyclazole), or mefenoxam.

In some embodiments, the biocide (for example a herbicide, bactericide, fungicide, pesticide, and/or insecticide) is a biocontrol agent. In such instances, the bacterial strain provided herein or active variant therof, and/or a composition derived therefrom is compatible with the biocontrol agent of interest. Biocontrol agents that can be used in the various methods and compositions disclosed herein include Bacillus amyloliquefaciens strain D747, (e.g. DOUBLENICKEL™ 55 or DOUBLENICKEL™ LC from Certis USA, L.L.C, having Accession No. FERM BP-8234 and described in U.S. Pat. No. 7,094,592, incorporated by reference in its entirety herein); Bacillus subtilis strain QST 713/AQ713 (e.g. SERENADE® MAX from Bayer CropScience LP, US, having NRRL Accession No. B-21661 and described in U.S. Pat. No. 6,060,051, incorporated by reference in its entirety herein; Bacillus subtilis strain AQ30002 (aka QST30002; NRRL Accession No. B-50421), and AQ30004 (aka QST3004; NRRL Accession No. B-50455), both from Bayer CropScience LP, US and described in U.S. Pat. No. 9,185,915, incorporated by reference in its entirety herein; Bacillus subtilis strain QST 713 (e.g. RHAPSODY® from Bayer CropScience, LP, US); Bacillus amyloliquefaciens strain GB03 (e.g. KODIAK® from Bayer CropScience, AG, DE); Bacillus subtilis strain FB17 (e.g. Veolondis™ from BASF); Bacillus pumilus strain QST 2808 (e.g. SONATA® from Bayer CropScience, having NRRL Accession No. B-30087); Bacillus pumilus strain GB34 (e.g. YIELD SHIELD from Bayer CropScience AG, DE); Bacillus subtilis var. amyloliquefaciens strain FZB24 (e.g. Taegro® from Syngenta); Bacillus finnus strain CNMC 1-1582 (e.g. VOTiVO® from Bayer CropScience); Streptomyces lydicus strain WYEC108 (e.g. ACTINOVATE® from Natural Industries, US, having ATCC Accession No. 55445): Streptomyces griseoviridis strain K61 (e.g. MYCOSTOP® from Verdera, cf. Crop Protection 2006, 25, 468-475, having Accession No. DSM 7206); Agrobacterium radiobacter strain 1026 (e.g. NOGALL™ from Becker Underwood, US); Agrobacterium radiobacter strain K84 (e.g. GALLTROL-A® from AgBioChem, CA); Pseudomonas fluorescens strain A506 (e.g. BLIGHTBAN® by NuFarm and also e.g. FROSTBAN B by Frost Technology Corp); Bacillus thuringiensis subspecies aizawai strain GC-91 (e.g. AGREE® from Certis USA, LLC); Bacillus thuringiensis subspecies kurstaki (e.g. BT 320 DUST from Wilbur-Ellis Company); Bacillus thuringiensis subspecies kurstaki strain EG7841 (e.g. CRYMAX® from Certis USA LLC); Bacillus thuringiensis subspecies kurstaki strain SA-12 (e.g. DELIVER® from Certis USA LLC); Bacillus thuringiensis subspecies kurstaki strain ABTS-351 (e.g. DiPel® from Valent BioSciences Corp, having ATCC Accession No. SD-1275); Bacillus thuringiensis subspecies kurstaki strain SA-11 (e.g. JAVELIN® from Certis USA LLC); Bacillus thuringiensis subspecies tenebrionis strain SA-10 (e.g. TRIDENT® from Certis USA LLC); Chromobacterium subtsugae strain PRAA4-1 (e.g. GRANDEVO® from Marrone BioInnovations, USA); Isaria fumosorosea Apopka Strain 97 (e.g. PFR-97™ from Certis USA LLC, having ATTC Accssion No. 20874); Burkholderia spp. strain A396 (e.g. VENERATE™ from Marrone BioInnovations, USA); Bacillus thuringiensis subspecies aizawai strain ABTS-1857 (e.g. XENTARI® from Valent BioSciences Corp, USA); a biologically pure strain of Pseudomonas fluorescens selected from ATCC 55171, ATCC 55170, ATCC 55169, ATCC 55175, ATCC 55174, and ATCC 55168 as described in U.S. Pat. No. 5,348,742, incorporated by reference herein; AIP1620 and AIP050999 as described in WO 2015/116838, incorporated by reference herein; AIP27511, AIP35174, AIP25773, AIP15251, AIP61892, AIP79428, AIP14931, AIP39589, and AIP36895 as described in WO 2017/040273, incorporated by reference herein; AIP011864, AIP060073, AIP089963, AIP098363, AIP054629, AIP038494, AIP064474, AIP085152, AIP004618, AIP037827, and AIP085784 as described in WO 2019/023226, incorporated by reference herein; AIP000648, AIP097852, and AIP051459 as described in WO 2019/074813, incorporated by reference herein; AIP031898, AIP023234, AIP024552, AIP035573, AIP071234, AIP080021, AIP001237, AIP050674, AIP071546, AIP049805, AIP016229, AIP081435, AIP082140, AIP000817, AIP060333, AIP070494, AIP015104, AIP011586, AIP010884, AIP082287, AIP088026, AIP065969, AIP018514, AIP033041, AIP092281, AIP081114, AIP046403, AIP022635, AIP070925, and AIP039063 as described in WO 2020/006555, incorporated by reference herein; AIP075655, AIP061382, and AIP029105 as described in WO 2020/077042, incorporated by reference herein; and AIP045885, AIP075655, AIP09474, AIP024525, AIP033287, AIP093798, AIP061639, AIP082862, AIP058187, AIP059286, and AIP036706 as described in WO 2020/092381, incorporated by reference herein.

In some embodiments, biocontrol agents that can be used in the various methods and compositions disclosed herein are pesticidal fungal strains. These fungal strains of interest include Trichoderma harzianum strain KRL-AG2 (also known as strain T-22, e.g. PLANTSHIELD® T-22G, ROOTSHIELD®, and TURFSHIELD from BioWorks, USA, having ATCC Accession No. 408479); Gliochladium vierns, aka Trichoderma virens, strain GL-21 (e.g. SOILGARD® 12G from Certis USA, L.L.C); Coniothyrium minitans strain CON/M/91-8 (e.g. CONTANS® from Encore Technologies, LLC, having Accession No. DSM-9660); Purpureocilium lilacinum; Ulocladium oudemansii U3 strain (aka HRU3 strain), (e.g. BOTRY-ZEN® by Botry-Zen Ltd, NZ); and Beauveria bassiana strain GHA (e.g. MYCOTROL® from Lam International Corp.).

In some embodiments, biocontrol agents that can be used in the various methods and compositions disclosed herein are pesticidal virus isolates. A virus may refer to a complete viral isolate itself or may refer to viral occlusion bodies. These viruses of interest include Cydia pomonella granulovirus (e.g. CYD-X® and CYD-X® HP, both from Certis USA LLC; CARPOVIRUSINE® from Sumitomo Corp.); Cydia pomonella granulovirus isolate V22 (e.g. MADEX HP from Andermatt Biocontrol AG); and polyhedral occlusion bodies of the nuclear polyhedrosis virus of Helicoverpa zea (e.g. GEMSTAR® from Certis USA LLC).

In some embodiments, biocontrol agents that can be used in the various methods and compositions disclosed herein are biopesticides derived from plant extracts. These biopesticides of interest include extracts of Chenopodium ambrosioides (e.g. Requiem® from Bayer CropScience LP).

In some embodiments, biocontrol agents that can be used in the various methods and compositions disclosed herein are biostimulants. A biostimulant is any microorganism or substance based on nautral resources. It is applied to plants, seeds, or the rhizosphere with the intention to stimulate natural processes of plants to benefit nutrient uptake, nutrient use efficiency, tolerance to abiotic stresses, and/or general plant health, including resistance to disease. Biostimulants include plant extracts, such as for example extracts derived from Reynoutria sachalinensis (e.g. REGALIA® from Marrone Bioinnovations, USA).

In some embodiments, a composition of the invention comprises bacterial strain AIP61892, or an active derivative of any thereof, or a spore, or a forespore or a combination of cells, forespores and/or spores from any one of AIP61892, or an active derivative of any thereof, or a spore, or a forespore or a combination of cells, forespores and/or spores from any one of AIP61892 or an active derivative of any thereof, and further comprises a biocide where the biocide is a bacterial strain. In further embodiments, the biocide is a Pseudomonas spp. In still further embodiments, the biocide is a biologically pure strain of Pseudomonas fluorescens selected from ATCC 55171, ATCC 55170, ATCC 55169, ATCC 55175, ATCC 55174, or ATCC 55168 as described in U.S. Pat. No. 5,348,742. In some embodiments, the biocide is bacterial strain AIP1620 or AIP050999 as described in WO 2015/116838. In further embodiments, the biocide is bacterial strain AIP1620, deposited as NRRL B-50897. In some embodiments, the composition is a stable formulation. In some embodiments, the composition is a tank mix.

In some embodiments of the invention, a kit of parts is provided comprising a bacterial strain provided herein or an active variant thereof, and/or a composition derived therefrom, and at least one biocide, in a spatially separated arrangement. In some embodiments, the biocide is an herbicide, fungicide, insecticide, bactericide, nematicide, pesticide, or other crop protection chemical. In some embodiments, the bacterial strain(s) and/or copper compound are comprised within a vessel(s), such as a box, bag, or bottle. In particular embodiments, the kit comprises AIP61892 or a variant thereof and a copper compound or AIP1620 or a variant thereof in a single vessel (e.g., box, bag, or bottle) with a partition between two compartments of the vessel, wherein the AIP61892 or a variant thereof is in one compartment and the copper compound or AIP1620 or a variant thereof is in the other compartment. The two compartments can each have a lid that can be opened or closed independently of the other. In some embodiments, the partition between the two compartments is removable to allow mixing of the two active ingredients. In some embodiments, the kit comprises instructions for use.

Non-limiting embodiments of the invention include:

1. A stable formulation comprising a biocontrol agent, wherein the biocontrol agent comprises:
(a) a bacterial strain deposited as NRRL No. B-67089; or
(b) a supernatant, fermentation product, filtrate, or extract derived from a whole cell culture of a bacterial strain deposited as NRRL No. B-67089.

2. The stable formulation of embodiment 1, wherein the formulation is a dry formulation or a liquid formulation.

3. The stable formulation of embodiment 2, wherein said dry formulation is dried to a water activity of 0.3 or less.

4. The stable formulation of any one of embodiments 1-3, wherein the formulation is a spray dried formulation, a wettable powder, or a granule.

5. The stable formulation of any one of embodiments 1-4, wherein the biocontrol agent is present at about 10⁵ CFU/gram to about 10¹² CFU/gram or at about 10⁵ CFU/ml to about 10¹² CFU/ml.

6. The stable formulation of any one of embodiments 1-5, wherein the formulation further comprises a pesticide, a fungicide, a bactericide, a nematicide, an insecticide, or an herbicide.

7. The stable formulation of embodiment 6, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

8. The stable formulation of any one of embodiments 1-7, wherein the formulation comprises a second biocontrol agent.

9. The stable formulation of embodiment 8, wherein the second biocontrol agent comprises Bacillus amyloliquefaciens strain D747, Bacillus subtilis strain QST 713, Bacillus amyloliquefaciens strain GB03, Bacillus subtilis strain FB17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

10. The stable formulation of embodiment 8, wherein the second biocontrol agent comprises a Pseudomonas spp.

11. The stable formulation of embodiment 10, wherein the second biocontrol agent comprises Pseudomonas fluorescens strain AIP1620.

12. The stable formulation of embodiment 11, wherein Pseudomonas fluorescens strain AIP1620 is present at about 10⁵ CFU/gram to about 10¹² CFU/gram, at about 10⁷ total cells/gram to about 10¹⁴ total cells/gram, at about 10⁵ CFU/ml to about 10¹² CFU/ml, or at about 10⁷ total cells/ml to about 10¹⁴ total cells/ml.

13. A coated seed comprising a seed and a coating on the seed, wherein the coating comprises a formulation comprising a biocontrol agent, wherein the biocontrol agent comprises a bacterial strain deposited as NRRL No. B-67089.

14. The coated seed of embodiment 13, wherein the biocontrol agent is present at about 10⁵ CFU/gram to about 10¹² CFU/gram or at about 10⁵ CFU/ml to about 10¹² CFU/ml.

15. The coated seed of any one of embodiments 13-14, wherein the coating further comprises a pesticide, a biocide, a fungicide, a bactericide, a nematicide, an insecticide or an herbicide.

16. The coated seed of any one of embodiments 13-14, wherein the fungicide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

17. The coated seed of any one of embodiments 13-16, wherein the coating comprises a second biocontrol agent.

18. The coated seed of embodiment 17, wherein the second biocontrol agent comprises Bacillus amyloliquefaciens strain D747, Bacillus subtilis strain QST 713, Bacillus amyloliquefaciens strain GB03, Bacillus subtilis strain FB17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

19. The coated seed of embodiment 17, wherein the second biocontrol agent comprises a Pseudomonas spp.

20. The coated seed of embodiment 19, wherein the second biocontrol agent comprises Pseudomonas fluorescens strain AIP1620.

21. The coated seed of embodiment 20, wherein Pseudomonas fluorescens strain AIP1620 is present at about 10⁵ CFU/gram to about 10¹² CFU/gram, at about 10⁷ total cells/gram to about 10¹⁴ total cells/gram, at about 10⁵ CFU/ml to about 10¹² CFU/ml, or at about 10⁷ total cells/ml to about 10¹⁴ total cells/ml.

22. A composition comprising an effective amount of a biocontrol agent, wherein the biocontrol agent comprises:
(a) a bacterial strain deposited as NRRL No. B-67089; or
(b) a supernatant, fermentation product, filtrate, or extract derived from a whole cell culture of a bacterial strain deposited as NRRL No. B-67089;
wherein said effective amount of said composition controls a plant pest and/or improves at least one agronomic trait of interest and/or improves plant health.

23. The composition of embodiment 22, wherein the bacterial strain is present in about 10⁵ CFU/gram to about 10¹² CFU/gram or at about 10⁵ CFU/ml to about 10¹² CFU/ml.

24. The composition of any one of embodiments 22-23, wherein the composition further comprises a pesticide, a biocide, a fungicide, a bactericide, a nematicide, an insecticide, or an herbicide.

25. The composition of embodiment 24, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

26. The composition of any one of embodiments 22-25, wherein the composition comprises a second biocontrol agent.

27. The composition of embodiment 26, wherein the second biocontrol agent comprises Bacillus amyloliquefaciens strain D747, Bacillus subtilis strain QST 713, Bacillus amyloliquefaciens strain GB03, Bacillus subtilis strain FB17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

28. The composition of embodiment 26, wherein the second biocontrol agent comprises a Pseudomonas spp.

29. The composition of embodiment 28, wherein the second biocontrol agent comprises Pseudomonas fluorescens strain AIP1620.

30. The composition of embodiment 29, wherein Pseudomonas fluorescens strain AIP1620 is present at about 10⁵ CFU/gram to about 10¹² CFU/gram, at about 10⁷ total cells/gram to about 10¹⁴ total cells/gram, at about 10⁵ CFU/ml to about 10¹² CFU/ml, or at about 10⁷ total cells/ml to about 10¹⁴ total cells/ml.

31. A method for controlling a plant pest comprising contacting said pest with an effective amount of the composition of any one of embodiments 22-29, or the formulation of any one of embodiments 1-12, wherein said composition or said formulation controls said plant pest.

32. The method of embodiment 31, wherein the plant pest is an insect, nematode, fungus, fungal-like organism, virus, viroid, bacterium, or a protozoan pathogen.

33. The method of any one of embodiments 31-32, wherein said plant pest comprises one or more bacterial pathogens selected from the group consisting of Acidovorax avenae, Burkholderia gladioli, Candidatus Liberibacter spp., Clavibacter michiganensis, Erwinia amylovora, Erwinia ananas, Erwinia chrysanthemi, Erwinia dissolvens, Erwinia herbicola, Erwinia rhapontic, Erwinia stewartii, Erwinia tracheiphila, Erwinia uredovora; Pectobacterium carotovorum, Pseudomonas syringae, Streptomyces scabies, Xanthomonas campestris, Xanthomonas axonopodis, Xanthomonas fragariae; Xanthomonas translucens, and Xylella fastidiosa.

34. The method of any one of embodiments 31-32, wherein said bacterial pathogen is Erwinia spp., Pseudomonas spp., or Xanthomonas spp.

35. The method of embodiment 34, wherein said bacterial pathogen is Erwinia amylovora or Xanthomonas campestris.

36. The method of embodiment 32, wherein said plant pest comprises one or more selected from the group consisting of Aspergillus spp., Botrytis spp., Cercospora spp., Alternaria spp., Didymella spp., Fusarium spp., Erysiphe spp., Colletotrichum spp., Monilinia spp., Mycosphaerella spp., Plasmopara spp., Peronospora spp., Pythium spp., Phytophthora spp., Phomopsis spp., Phakopsora spp., Podosphaera spp., Rhizopus spp., Rhizoctonia spp., Sclerotium spp., Sclerotinia spp., Uncinula spp., Venturia spp., Wilsonomyces spp., and Plasmodiophora spp.

37. The method of embodiment 32, wherein said plant pest comprises one or more selected from the group consisting of Aspergillus favus, Botrytis cinerea, Cercospora sojina, Alternaria solani, Colletotrichum acutatum, Colletotrichum cereal, Colletotrichum sublineolum, Didymella bryoniae, Erysiphe necator, Fusarium graminearum, Fusarium solani, Fusarium oxysporum, Monilinia fructicola, Monilinia laxa, Monilinia fructigena, Mycosphaerella citri, Mycosphaerella fijiensis, Podosphaera xanthii, Plasmopara viticola, Plasmodiophora brassicae, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Phakopsora pachyrizi, Rhizoctonia solani, Rhizopus stolonifera, Sclerotium rolfsii, Sclerotinia minor, Sclerotinia sclerotiorium, Uncinula necator, and Venturia inaequalis.

38. The method of any one of embodiments 31-37, wherein the method further comprises contacting said pest with a pesticide, a fungicide, a bactericide, a nematicide, an insecticide, or an herbicide.

39. The method of embodiment 38, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

40. The method of any one of embodiments 31-39, wherein the method further comprises contacting said pest with a second biocontrol agent.

41. The method of embodiment 40, wherein the second biocontrol agent comprises Bacillus amyloliquefaciens strain D747, Bacillus subtilis strain QST 713, Bacillus amyloliquefaciens strain GB03, Bacillus subtilis strain FB17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

42. The method of embodiment 40, wherein the second biocontrol agent comprises a Pseudomonas spp.

43. The method of embodiment 42, wherein the second biocontrol agent comprises Pseudomonas fluorescens strain AIP1620.

44. The method of any one of embodiments 40-43, wherein the biocontrol agent and the second biocontrol agent are applied simultaneously.

45. The method of any one of embodiments 40-43, wherein the biocontrol agent and the second biocontrol agent are applied sequentially.

46. A method for growing a plant susceptible to a plant pest comprising applying to a plant, a plant part, a seed, or an area of cultivation a biocontrol agent, wherein the biocontrol agent comprises:
(a) an effective amount of a bacterial strain deposited as NRRL No. B-67089; or
(b) an effective amount of a supernatant, fermentation product, filtrate, or extract derived from a whole cell culture of a bacterial strain deposited as NRRL No. B-67089;
wherein said effective amount controls a plant pest.

47. A method of controlling a plant pest comprising applying to a plant, a plant part, a seed, or an area of cultivation a biocontrol agent, wherein the biocontrol agent comprises:
(a) an effective amount of a bacterial strain deposited as NRRL No. B-67089; or
(b) an effective amount of a supernatant, fermentation product, filtrate, or extract derived from a whole cell culture of a bacterial strain deposited as NRRL No. B-67089;
wherein said effective amount controls the plant pest.

48. The method of embodiment 46 or 47, wherein said effective amount of the bacterial strain comprises at least about 10⁴ to 10¹⁶ colony forming units (CFU) per hectare.

49. The method of any one of embodiments 46-48, wherein the method further comprises applying an effective amount of a pesticide, a fungicide, a bactericide, a nematicide, an insecticide, or an herbicide.

50. The method of embodiment 49, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

51. The method of any one of embodiments 46-50, wherein a second biocontrol agent is applied at an effective amount.

52. The method of embodiment 51, wherein the second biocontrol agent comprises Bacillus amyloliquefaciens strain D747, Bacillus subtilis strain QST 713, Bacillus amyloliquefaciens strain GB03, Bacillus subtilis strain FB17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

53. The method of embodiment 51, wherein the second biocontrol agent comprises a Pseudomonas spp.

54. The method of embodiment 53, wherein the second biocontrol agent comprises Pseudomonas fluorescens strain AIP1620.

55. The method of embodiment 54, wherein the effective amount of Pseudomonas fluorescens strain AIP1620 comprises at least about 10⁴ to 10¹⁶ colony forming units (CFU) per hectare or at least about 10⁶ to 10¹⁸ total cells per hectare.

56. The method of any one of embodiments 51-55, wherein the biocontrol agent and the second biocontrol agent are applied simultaneously.

57. The method of any one of embodiments 51-55, wherein the biocontrol agent and the second biocontrol agent are applied sequentially.

58. The method of any one of embodiments 46-57, wherein the plant pest is an insect, nematode, fungus, fungal-like organism, virus, viroid, bacterium, or a protozoan pathogen.

59. The method of any one of embodiments 46-57, wherein said plant pest comprises one or more bacterial pathogens selected from the group consisting of Acidovorax avenae, Burkholderia gladioli, Candidatus Liberibacter spp., Erwinia amylovora, Erwinia ananas, Erwinia chrysanthemi, Erwinia dissolvens, Erwinia herbicola, Erwinia rhapontic, Erwinia stewartii, Erwinia tracheiphila, Erwinia uredovora; Pectobacterium carotovorum, Pseudomonas syringae, Streptomyces scabies, Xanthomonas campestris, Xanthomonas axonopodis, Xanthomonas fragariae; Xanthomonas translucens, and Xylella fastidiosa.

60. The method of embodiment 58, wherein said bacterial pathogen is Erwinia spp., Pseudomonas spp., or Xanthomonas spp.

61. The method of embodiment 60, wherein said bacterial pathogen is Erwinia amylovora or Xanthomonas campestris.

62. The method of any one of embodiments 46-57, wherein said plant pest comprises one or more selected from the group consisting of Aspergillus spp., Botrytis spp., Cercospora spp., Alternaria spp., Didymella spp., Fusarium spp., Erysiphe spp., Colletotrichum spp., Monilinia spp., Mycosphaerella spp., Plasmopara spp., Peronospora spp., Pythium spp., Phytophthora spp., Phomopsis spp., Phakopsora spp., Podosphaera spp., Rhizopus spp., Rhizoctonia spp., Sclerotium spp., Sclerotinia spp., Uncinula spp., Venturia spp., Wilsonomyces spp., and Plasmodiophora spp.

63. The method of embodiment 62, wherein said plant pest comprises one or more selected from the group consisting of Aspergillus favus, Botrytis cinerea, Cercospora sojina, Alternaria solani, Colletotrichum acutatum, Colletotrichum cereal, Colletotrichum sublineolum, Didymella bryoniae, Erysiphe necator, Fusarium graminearum, Fusarium solani, Fusarium oxysporum, Monilinia fructicola, Monilinia laxa, Monilinia fructigena, Mycosphaerella citri, Mycosphaerella fijiensis, Podosphaera xanthii, Plasmopara viticola, Plasmodiophora brassicae, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Phakopsora pachyrizi, Rhizoctonia solani, Rhizopus stolonifera, Sclerotium rolfsii, Sclerotinia minor, Sclerotinia sclerotiorium, Uncinula necator, and Venturia inaequalis.

64. The method of any one of embodiments 46-63, wherein the biocontrol agent is applied to the plant or plant part prior to harvest.

65. The method of any one of embodiments 46-63, wherein the biocontrol agent is applied to the plant or plant part after harvest.

66. The method of embodiment 65, wherein the biocontrol agent is applied by dipping, drenching, flooding, fogging, spraying, dusting, or injecting.

67. A method of treating or preventing a plant disease comprising applying to a plant or plant part a plant, a plant part, a seed, or an area of cultivation a biocontrol agent, wherein the biocontrol agent comprises:
(a) an effective amount of a biocontrol agent comprising a bacterial strain deposited as NRRL No. B-67089; or
(b) an effective amount of a supernatant, fermentation product, filtrate, or extract derived from a whole cell culture of a bacterial strain deposited as NRRL No. B-67089;
wherein the effective amount treats or prevents the plant disease.

68. The method of embodiment 67, wherein said effective amount of the bacterial strain comprises at least about 10⁴ to 10¹⁶ CFU per hectare.

69. The method of embodiment 67 or 68, wherein the plant disease is Asian Soybean Rust (ASR), gray mold, leaf spot, Frogeye Leaf Spot, Early Blight, Damping off complex, Brown Patch, black scurf, root rot, belly rot, Sheath Blight, Powdery Mildew, Anthracnose, Black Sigatoka, Anthracnose leaf spot, Downy Mildew, Pythium Blight, Late Blight, Fusarium Head Blight, sudden death syndrome (SDS), Fusarium Wilt, Corn Stalk Rot, Brown Rust, Black Rust, Yellow Rust, Wheat Rust, Rust, Apple Scab, Post-bloom Fruit Drop, Gummy Stem Blight, Greasy Spot, Corn Stalk Rot, Cherry Blossom Blight, Damping Off, Fire Blight, Citrus Greening Disease, Clubroot, Verticillium Wilt, Rhizopus Rot, Bacterial Spot, or Brown Rot.

70. The method of any one of embodiments 67-69, wherein the method further comprises applying an effective amount of a pesticide, a fungicide, a bactericide, a nematicide, an insecticide, or an herbicide.

71. The method of embodiment 70, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

72. The method of any one of embodiments 67-71, wherein a second biocontrol agent is applied at an effective amount.

73. The method of embodiment 72, wherein the second biocontrol agent comprises Bacillus amyloliquefaciens strain D747, Bacillus subtilis strain QST 713, Bacillus amyloliquefaciens strain GB03, Bacillus subtilis strain FB17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

74. The method of embodiment 72, wherein the second biocontrol agent comprises a Pseudomonas spp.

75. The method of embodiment 74, wherein the second biocontrol agent comprises Pseudomonas fluorescens strain AIP1620.

76. The method of embodiment 75, wherein the effective amount of Pseudomonas fluorescens strain AIP1620 comprises at least about 10⁴ to 10¹⁶ colony forming units (CFU) per hectare or at least about 10⁶ to 10¹⁸ total cells per hectare.

77. The method of any one of embodiments 72-76, wherein the biocontrol agent and the second biocontrol agent are applied simultaneously.

78. The method of any one of embodiments 72-76, wherein the biocontrol agent and the second biocontrol agent are applied sequentially.

79. The method of any one of embodiments 67-78, wherein the biocontrol agent is applied to the plant or plant part after harvest.

80. The method of embodiment 79, wherein the biocontrol agent is applied by dipping, drenching, flooding, fogging, spraying, dusting, or injecting.

81. A method for improving at least one agronomic trait of interest in a plant and/or improving the health of a plant comprising applying to said plant an effective amount of the composition of any one of embodiments 22-30, or the formulation of any one of embodiments 1-12, wherein said composition or said formulation improves at least one agronomic trait of interest of said plant and/or improves the health of said plant when compared to a plant to which the composition or formulation was not applied.

82. A method for improving at least one agronomic trait of interest in a plant and/or improving the health of a plant comprising applying to a plant, a plant part, a seed, or an area of cultivation a biocontrol agent, wherein the biocontrol agent comprises:
(a) an effective amount of a bacterial strain deposited as NRRL No. B-67089; or
(b) an effective amount of a supernatant, fermentation product, filtrate, or extract derived from a whole cell culture of a bacterial strain deposited as NRRL No. B-67089;
wherein said effective amount improves at least one agronomic trait of interest of said plant and/or improves the health of said plant when compared to a plant to which the biocontrol agent was not applied.

83. The method of embodiment 82, wherein said effective amount of the bacterial strain comprises at least about 10⁴ to 10¹⁶ colony forming units (CFU) per hectare.

84. The method of embodiment 82 or 83, wherein a second biocontrol agent is applied at an effective amount.

85. The method of embodiment 84, wherein the second biocontrol agent comprises a Pseudomonas spp.

86. The method of embodiment 85, wherein the second biocontrol agent comprises Pseudomonas fluorescens strain AIP1620.

87. The method of embodiment 86, wherein the effective amount of Pseudomonas fluorescens strain AIP1620 comprises at least about 10⁴ to 10¹⁶ colony forming units (CFU) per hectare or at least about 10⁶ to 10¹⁸ total cells per hectare.

88. The method of any one of embodiments 84-87, wherein the biocontrol agent and the second biocontrol agent are applied simultaneously.

89. The method of any one of embodiments 84-87, wherein the biocontrol agent and the second biocontrol agent are applied sequentially.

90. The method of any one of embodiments 54-66, wherein control of said plant pest is increased synergistically.

91. The method of embodiment 90, wherein said plant pest is selected from:
a. Rhizoctonia, such as Rhizoctonia solani;
b. Fusarium, such as Fusarium oxysporum;
c. Phytophthora, such as Phytophthora nicotianae; and
d. Phythium, such as Pythium ultimum.

The following examples are offered by way of illustration and not by way of limitation.

### EXAMPLES

### Example 1: Efficacy of AIP61892 Against Erwinia amylovora

### Example 1.1: Field Trial 1, New York State

An apple field trial was performed in New York state to test the efficacy of AIP61892 on fire blight disease caused by the bacteria Erwinia amylovora. Agrimycin® 17 (Nufarms Americas, Inc), which comprises the antibiotic streptomycin, was used as a control. Formulated AIP61892 with a minimum of 1×10⁹ CFU/g was tested at the rate of 3 pounds per acre. Each treatment was applied at three points in the trial: the pink bud stage, full bloom, and petal fall. All treatments were applied with a non-ionic surfactant adjuvant. Visual ratings of the number of fire blight strikes per tree were made 113 days after the first application. Data were analyzed using R and ARM software. Means were compared with a Least Significant Difference test at the p=0.05 level. Statistically significant differences between means are indicated by different letters following the mean value of each treatment in Table 2.

TABLE 2
Efficacy of AIP61892 against Apple Fire
Blight in a Field Trial in New York State

| | | Average Number of Fire |
|---|---|---|
| Treatment | Rate | Blight Strikes per Tree |
| Untreated | | 22.8 a |
| AIP61892 | 3 lb/A | 5.8 b |
| Agrimycin 17 | 5 lb/A | 3.3 b |

AIP61892 and Agrimycin® 17 treatments demonstrated a statistically significant reduction in the number of fire blight strikes compared to the untreated plots. The reduction in the number of trees exhibiting fire blight symptoms was statistically similar for AIP61892 and Agrimycin® 17 treatments.

### Example 1.2: Field Trial 2, Washington State

An apple field trial was performed in Washington state to test the efficacy of AIP61892 on fire blight disease caused by the bacteria Erwinia amylovora. AIP61892 was compared to a standard commercial antibiotic program consisting of Mycoshield® which comprises the antibiotic oxytetracycline; Kasumin® which comprises the antibiotic kasugamycin; and Agrimycin® 17 which comprises the antibiotic streptomycin. Formulated AIP61892 with a minimum of 1×10⁹ CFU/g was tested at the rate of 3 pounds per acre. Each treatment was applied at three points in the trial: the full bloom stage, petal fall, and 11 days after petal fall. All treatments were applied with a non-ionic surfactant adjuvant. Visual ratings of the percent severity of fire blight were made 81 days after the last application. Data were analyzed using R and ARM software. Means were compared with a Least Significant Difference test at the p=0.05 level. Statistically significant differences between means are indicated by different letters following the mean value of each treatment in Table 3.

TABLE 3
Efficacy AIP61892 against Apple Fire Blight
in a Field Trial in Washington State

| | | Average Percent Severity |
|---|---|---|
| Treatment | Rate | of Fire Blight |
| Untreated | | 8.8 a |
| AIP61892 | 3 lb/A | 5.0 b |
| Standard Program | | 1.3 b |

AIP61892 and the standard program treatments demonstrated statistically significant reduction in the severity of fire blight over the untreated plots. The reduction in the severity of fire blight symptoms was statistically similar for the AIP61892 and standard program treatments.

### Example 2: Efficacy of AIP61892 Against Xanthomonas capestris

### Example 2.1: Tomato Field Trial 1, Georgia

A tomato field trial was performed in Georgia to test the efficacy of AIP61892 on tomato bacterial spot disease caused by Xanthomonas campestris pv. vesicatoria. Cueva® (Certis USA, LLC) was used as a control and also in a rotation with formulated AIP61892. Formulated AIP61892 with a minimum of 1×10⁹ CFU/g was tested at rates of 2 pounds of product per acre, 3 pounds of product per acre and 5 pounds of product per acre compared to Cueva® at a rate of 2 gallons product per 100 gallons of water. All treatments were applied with a non-ionic surfactant adjuvant. There were six applications of each treatment in the trial, applied at a 7 day intervals. In the rotation treatments, Cueva® was applied at 2 gallons of product per 100 gallons of water at the first, third and fifth application timings. Formulated AIP61892 was applied at the second, fourth and sixth application timings at a rate of 3 pounds of product per acre in one rotation treatment and a rate of 5 pounds of product per acre in a second rotation treatment. Treatment applications began prior to disease infection. Each treatment was applied on a 7 day interval, beginning on April 30. Visual ratings of percent severity of bacterial spot were collected on a per plot basis. The percent severity ratings of bacterial spot shown in Table 3 were collected on June 1, 21 days after the first application of each treatment. Data were analyzed using R and ARM software. Means were compared with a Least Significant Difference test at the p=0.05 level. Statistically significant differences between means are indicated by different letters following the mean value of each treatment in Table 4.

TABLE 4
Efficacy of AIP61892 on Tomato Bacterial
Spot in a Field Trial in Georgia

| | | Average Percent |
|---|---|---|
| | | Severity of |
| | | Bacterial Spot |
| | | (21 days after |
| Treatment | Rate | first application) |
| Untreated | | 21.8 a |
| AIP61892 | 2 lb/A | 8.5 bc |
| AIP61892 | 3 lb/A | 1.8 d |
| AIP61892 | 5 lb/A | 1 d |
| Cueva ® | 2 gal/100 gal water | 11 b |
| Cueva ® (apps ACE)/ | 2 gal/100 gal water/3 lb/A | 2 d |
| AIP61892 (apps BDF) | | |
| Cueva ® (apps ACE)/ | 2 gal/100 gal water/5 lb/A | 1.3 d |
| AIP61892 (apps BDF) | | |

All treatment rates of AIP61892 alone demonstrated statistically significant reduction in the severity of bacterial spot over the untreated plots. The 3 pound and 5 pound rates of AIP61892 alone were statistically better at reducing disease severity than the copper product Cueva® alone. When AIP61892 was added in a rotation with Cueva®, the severity of disease was significantly reduced compared to the Cueva® treatment alone.

### Example 2.2: Tomato Field Trial 2, North Carolina

A tomato field trial was performed in North Carolina to test the efficacy of AIP61892 on tomato bacterial spot disease caused by Xanthomonas campestris pv. vesicatoria. Cueva® was used as a control and also in a rotation with formulated AIP61892. Formulated AIP61892 with a minimum of 1×10⁹ CFU/g was tested at rates of 2 pounds of product per acre, 3 pounds of product per acre and 5 pounds of product per acre compared to Cueva® at a rate of 2 gallons product per 100 gallons of water. All treatments were applied with a non-ionic surfactant adjuvant. There were ten applications of each treatment in the trial, applied at 7 day intervals. In the rotation treatments, Cueva® was applied at 2 gallons of product per 100 gallons of water at the first, third, fifth, seventh and nineth application timings. Formulated AIP61892 was applied at the second, fourth, sixth, eighth and tenth application timings at a rate of 3 pounds of product per acre in one rotation treatment and a rate of 5 pounds of product per acre in a second rotation treatment. Treatment applications began prior to disease infection. Each treatment was applied on a 7 day interval, beginning on August 26. Visual ratings of percent severity of bacterial spot were collected on a per plot basis. The percent severity ratings of bacterial spot shown in Table 4 were collected on October 14, 49 days after the first application of treatments. Data were analyzed using R and ARM software. Means were compared with a Least Significant Difference test at the p=0.05 level. Statistically significant differences between means are indicated by different letters following the mean value of each treatment in Table 5.

TABLE 5
Efficacy of AIP61892 on Tomato Bacterial
Spot in a Field Trial in North Carolina

| | | Average Percent |
|---|---|---|
| | | Severity of |
| | | Bacterial Spot |
| | | (49 days after |
| Treatment | Rate | application A) |
| Untreated | | 27.3 a |
| AIP61892 | 2 lb/A | 19.3 b |
| AIP61892 | 3 lb/A | 21 b |
| AIP61892 | 5 lb/A | 19.3 b |
| Cueva ® | 2 gal/100 gal water | 20 b |
| Cueva ® (apps ACE)/ | 2 gal/100 gal water/3 lb/A | 18.3 b |
| AIP61892 (apps BDF) | | |
| Cueva ® (apps ACE)/ | 2 gal/100 gal water/5 lb/A | 17.3 b |
| AIP61892 (apps BDF) | | |

All treatment rates of AIP61892 alone demonstrated statistically significant reduction in the seventy of bacterial spot over the untreated plots. The 2 pound, 3 pound and 5 pound rates of AIP61892 alone were similar to the copper product Cueva® at reducing disease severity.

### Example 2.3: Pepper Field Trial, North Carolina

A bell pepper field trial was performed in North Carolina to test the efficacy of AIP61892 on pepper bacterial spot disease caused by Xanthomonas campestris pv. vesicatoria. Cueva® was used as a control and also in a rotation with formulated AIP61892. Formulated AIP61892 with a minimum of 1×10⁹ CFU/g was tested at rates of 2 pounds of product per acre, 3 pounds of product per acre and 5 pounds of product per acre compared to Cueva® at a rate of 2 gallons product per 100 gallons of water. All treatments were applied with a non-ionic surfactant adjuvant. There were eight applications of each treatment in the trial on a 7 day interval. In the rotation treatments, Cueva® was applied at 2 gallons of product per 100 gallons of water at the first, third, fifth and seventh and application timings. Formulated AIP61892 was applied at the second, fourth, sixth and eighth application timings at a rate of 3 pounds of product per acre in one rotation treatment and a rate of 5 pounds of product per acre in a second rotation treatment. Treatment applications began prior to disease infection. Each treatment was applied on a 7 day interval, beginning on June 24. Visual ratings of percent severity of bacterial spot were collected on a per plot basis. The percent severity ratings of bacterial spot shown in Table 6 were collected on August 20, 57 days after the first application of treatments. Data were analyzed using R and ARM software. Means were compared with a Least Significant Difference test at the p=0.05 level. Statistically significant differences between means are indicated by different letters following the mean value of each treatment in Table 5.

TABLE 6
Efficacy of AIP61892 on Pepper Bacterial
Spot in a Field Trial in North Carolina

| | | Average Percent |
|---|---|---|
| | | Severity of |
| | | Bacterial Spot |
| | | (57 days after |
| Treatment | Rate | application A) |
| Untreated | | 63 a |
| AIP61892 | 2 lb/A | 44.8 bc |
| AIP61892 | 3 lb/A | 42.5 c |
| AIP61892 | 5 lb/A | 37.8 d |
| Cueva ® | 2 gal/100 gal water | 48 b |
| Cueva ® (apps ACE)/ | 2 gal/100 gal water/3 lb/A | 43.5 bc |
| AIP61892 (apps BDF) | | |
| Cueva ® (apps ACE)/ | 2 gal/100 gal water/5 lb/A | 38 d |
| AIP61892 (apps BDF) | | |

All treatment rates of AIP61892 alone demonstrated statistically significant reduction in the severity of bacterial spot over the untreated plots. The 2 pound rate of AIP61892 alone was similar statistically to the copper product Cueva® at reducing disease severity. The 3 pound and 5 pound rates of AIP61892 alone were significantly better than the copper product Cueva® at reducing disease severity. When AIP61892 at 5 pounds was added in a rotation with Cueva®, the severity of disease was significantly reduced compared to the Cueva® treatment alone.

### Example 3: AIP61892 and AIP1620 Applications in Combination Against Powdery Mildew of Grape

Grape field trials were performed in Washington state and New York state to test the efficacy of the combination of AIP61892 and AIP1620 (AIP1620 as described in WO 2015/116838; deposited as NRRL B-50897) against powdery mildew of grapes (Uncinula necator). A liquid formulation of AIP61892 (with a minimum of 1.30×10¹⁰ CFU/ml), and a formulation of AIP1620 (with a minimum of 1×10⁶ CFU/g) were mixed together in a spray tank at the time of application. A treatment of the liquid formulation of AIP61892 alone was also tested at a rate of 5 pints per acre in each trial. There were two combination AIP61892 and AIP1620 treatments in each trial. The first combination treatment was formulated AIP1620 at a rate of 2.5 pounds per acre combined with the liquid formulation of AIP61892 at a rate of 2.5 pints per acre. The second combination treatment was formulated AIP1620 at a rate of 2.5 pounds per acre combined with the liquid formulation of AIP61892 at a rate of 5 pints per acre. Each trial consisted of 4 replicates in a randomized complete block design. Both trial sites were naturally infected with grape powdery mildew (Uncinula necator) during the trial period. Treatment applications for powdery mildew began in mid-May and continued on a 7-10 day reapplication interval throughout the season. Disease incidence and severity of powdery mildew were evaluated periodically using a visual 0-100% scale. Results across plots were averaged to account for natural variability. Treated plots were compared to untreated controls and the percent control for each treatment was calculated using the formula:

$$\% Control = ( ( 1 - ( disease in treated plot / disease in control plot ) ) * 100$$

Disease incidence of powdery mildew on fruit was recorded and the mean results of the field trial in Washington state are shown in Table 7. The active ingredient is AIP61892 or AIP1620. Results of field trial 1 and field trial 2 in New York state are shown in Tables 8 and 9, respectively.

TABLE 7
Efficacy of AIP61892 and AIP61892 + AIP1620 on Powdery
Mildew of Grapes in a Field Trial in Washington state

| | Dose Rate | Dose Rate | % |
|---|---|---|---|
| Treatment | (product) | (active ingredient) | Control |
| AIP61892 | 5 pt/A | 1.3 pt active/A | 67 |
| AIP61892 + | 2.5 pt/A + | 0.65 pt active/A + | 79 |
| AIP1620 | 2.5 lb/A | 1.75 lb active/A | |
| AIP61892 + | 5 pt/A + | 1.3 pt active/A + | 73 |
| AIP1620 | 2.5 lb/A | 1.75 lb active/A | |

TABLE 8
Efficacy of AIP61892 and AIP61892 + AIP1620 on Powdery
Mildew of Grapes in Field Trial 1 in New York state

| | Dose Rate | Dose Rate | % |
|---|---|---|---|
| Treatment | (product) | (active ingredient) | Control |
| AIP61892 | 5 pt/A | 1.3 pt active/A | 58 |
| AIP61892 + | 2.5 pt/A + | 0.65 pt active/A + | 58 |
| AIP1620 | 2.5 lb/A | 1.75 lb active/A | |
| AIP61892 + | 5 pt/A + | 1.3 pt active/A + | 68 |
| AIP1620 | 2.5 lb/A | 1.75 lb active/A | |

TABLE 9
Efficacy of AIP61892 and AIP61892 + AIP1620 on Powdery
Mildew of Grapes in Field Trial 2 in New York state

| | Dose Rate | Dose Rate | % |
|---|---|---|---|
| Treatment | (product) | (active ingredient) | Control |
| AIP61892 | 5 pt/A | 1.3 pt active/A | 63 |
| AIP61892 + | 2.5 pt/A + | 0.65 pt active/A + | 69 |
| AIP1620 | 2.5 lb/A | 1.75 lb active/A | |
| AIP61892 + | 5 pt/A + | 1.3 pt active/A + | 71 |
| AIP1620 | 2.5 lb/A | 1.75 lb active/A | |

### Example 4: AIP61892 and AIP1620 Applications in Combination Against Sclerotium rolfsii on Tomatoes

A tomato greenhouse trial was performed to test the efficacy of AIP61892 and AIP1620 against the soil pathogen Sclerotium rolfsii. AIP61892 and AIP1620 alone and in tank mix combination were compared to the synthetic fungicide Fontelis® (penthiopyrad; Corteva™ agriscience). AIP1620 was also tank mixed with Fontelis®. The testing rate for solo AIP1620 was 5 pounds formulated AIP1620 (minimum 1×10⁶ CFU/g) per acre, while the testing rate for solo AIP61892 was 3 pounds formulated AIP61892 (minimum 1×10⁹ CFU/g) per acre. The AIP1620+AIP61892 tank mix contained 2.5 pounds product per acre of formulated AIP1620 plus 1.5 pounds product per acre of formulated AIP61892. The AIP1620+Fontelis® tank mix contained 2.5 pounds product per acre of formulated AIP1620 plus 16 ounces product per acre of Fontelis®. The solo treatment of Fontelis® was tested at a rate of 24 ounces product per acre.

Pots of soil were prepared by making a hole in the soil for each transplant and adding inoculum of S. rolfsii to each hole with the exception of the non-inoculated check treatment. Tomato seedlings were transplanted with one plant per soil hole and the soil was closed around each plant. Immediately after transplanting, a single drench application of each treatment was applied to the soil around the plants. The number of living plants were counted and recorded for each treatment every seven days for a total of 28 days after transplanting. Data were analyzed using R and ARM software. Means were compared with a Least Significant Difference test at the p=0.05 level. Statistically significant differences between means are indicated by different letters following the mean value of each treatment in Table 10.

TABLE 10
Efficacy of AIP61892 and AIP1620 on Sclerotium rolfsii
in a Greenhouse Trial

| | | | Average Percent Incidence |
|---|---|---|---|
| | | | of Dead Plants |
| Treatment | Rate | | (28 days after application) |
| Non-Inoculated Check | | | 0 a |
| Inoculated Check | | | 78 c |
| AIP1620 | 5 | lb/A | 33 b |
| AIP61892 | 3 | lb/A | 8 a |
| AIP1620 + AIP61892 | 2.5 lb/A + 1.5 lb/A | | 3.3 a |
| AIP1620 + Fontelis ® | 2.5 lb/A + 16 oz/A | | 0 a |
| Fontelis ® | 24 | oz/A | 33 b |

All treatments improved the survival of tomato plants compared to the inoculated check. The absence of dead plants in the non-inoculated check served as a negative control and indicates the effect of introducing the S. rolfsii inoculum into the other treatments. The three most effective treatments were the AIP61892 treatment, the tank mix of AIP1620+AIP61892 and the tank mix of AIP1620+Fontelis®.

### Example 5: AIP61892 and AIP1620 Applications in Combination Increase Plant Vigor

The previously described tomato greenhouse trial in Example 4 was also evaluated for plant vigor every 7 days for 28 days after transplanting. The evaluations were made using a visual 1-5 rating scale where a rating of 5 indicates the most vigorous plants and 1 indicates the least plant vigor. Data were analyzed using R and ARM software. Means were compared with a Least Significant Difference test at the p=0.05 level. Statistically significant differences between means are indicated by different letters following the mean value of each treatment in Table 11.

TABLE 11
Effects of AIP61892 and AIP1620 on Tomato
Plant Vigor in a Greenhouse Trial

| | | | Average Plant Vigor |
|---|---|---|---|
| | | | on a 1-5 Scale |
| Treatment | Rate | | (28 days after application) |
| Non-Inoculated Check | | | 2.5 c |
| Inoculated Check | | | 1.5 d |
| AIP1620 | 5 | lb/A | 3 c |
| AIP61892 | 3 | lb/A | 5 a |
| AIP1620 + AIP61892 | 2.5 lb/A + 1.5 lb/A | | 5 a |
| AIP1620 + Fontelis ® | 2.5 lb/A + 16 oz/A | | 3.8 b |
| Fontelis ® | 24 | oz/A | 3 c |

The plants in the inoculated check treatment were significantly less vigorous than the plants in the non-inoculated check. The vigor of the inoculated AIP1620 solo treatment and the inoculated Fontelis® solo treatment were statistically similar to the non-inoculated check treatment indicating an improvement in vigor related to disease control. The inoculated AIP61892 solo and AIP1620+AIP61892 tank mix treatments demonstrated statistically significant levels of vigor above that of the non-inoculated check, suggesting a biostimulant effect that increases tomato plant health.

### Example 6: Efficacy of AIP61892 Against Pseudomonas syringae, Blueberry Field Trial

A blueberry field trial was performed in Oregon to test the efficacy of AIP61892 on bacterial canker/blight caused by Pseudomonas syringae. Copper-Count-N® (Mineral Research & Development) (10 quarts) and SERENADE® Opti (Bayer Crop Science LP, US) (1 pound of product per acre) were used as controls. Formulated AIP61892 was tested at rates of 2 pounds of product per acre, 3 pounds of product per acre, and 5 pounds of product per acre. There were six applications of each treatment in the trial, applied at 22-30 day intervals. Treatment applications began prior to disease infection. Visual ratings of percent severity of bacterial blight were collected on a per plot basis. The percent severity ratings of bacterial blight shown in Table 12 were collected 106 days after the first application of each treatment. Data were analyzed using R and ARM software. Means were compared with a Least Significant Difference test at the p=0.05 level. Statistically significant differences between means are indicated by different letters following the mean value of each treatment in Table 12. AIP61892 performed better than Copper-Count-N® at controlling bacterial canker caused by Pseudomonas syringae.

TABLE 12
Efficacy of AIP61892 on Blueberry Bacterial
Blight in a Field Trial in Georgia

| | | Average Percent Severity |
|---|---|---|
| | | of Bacterial Blight |
| | | (106 days after first |
| Treatment | Rate | application) |

| | | | |
|---|---|---|---|
| Untreated | | | 17 a |
| AIP61892 | 2 | lb/A | 11.8 c |
| AIP61892 | 3 | lb/A | 12.5 bc |
| AIP61892 | 5 | lb/A | 11 c |
| Copper-Count-N ® | 10 | qt | 15.8 ab |
| SERENADE ® Opti | 1 | lb/A | 12.8 bc |

### Example 7: Efficacy of AIP61892 Against Pseudomonas viridiflava, Onion Field Trial

An onion field trial was performed in Georgia to test the efficacy of AIP61892 on bacterial streak caused by Pseudomonas viridiflava. Kocide® 3000 (DuPont™) (1.5 pound of product per acre) and SERENADE® Opti (Bayer Crop Science LP, US) (1 pound of product per acre) were used as controls. Formulated AIP61892 was tested at rates of 1 pound of product per acre, 2 pounds of product per acre, and 3 pounds of product per acre. There were 14 applications of each treatment in the trial, applied at 4-5 day intervals. In the rotation treatments, Kocide® 3000 was applied at 1.5 pound per acre and formulated AIP61892 was applied at a rate of 1 pound of product per acre in one rotation treatment and a rate of 2 pounds of product per acre in a second rotation treatment. Treatment applications began prior to disease infection. Visual ratings of percent severity of bacterial streak were collected on a per plot basis. The percent severity ratings of bacterial streak shown in Table 13 were collected 20 days after the first application of each treatment. Data were analyzed using R and ARM software. Means were compared with a Least Significant Difference test at the p=0.05 level. Statistically significant differences between means are indicated by different letters following the mean value of each treatment in Table 13. AIP61892 alone has activity on Pseudomonas viridiflava and also works well in rotation with the copper product Kocide® 3000.

TABLE 13
Efficacy of AIP61892 on Onion Bacterial
Streak in a Field Trial in Oregon

| | | | Average Percent Severity |
|---|---|---|---|
| | | | of Bacterial Spot |
| | | | (21 days after first |
| Treatment | Rate | | application) |
| Untreated | | | 17.5 a |
| AIP61892 | 1 | lb/A | 12.8 b |
| AIP61892 | 2 | lb/A | 10 c |
| AIP61892 | 3 | lb/A | 7.8 cd |
| SERENADE ® Opti | 1 | lb/A | 10 c |
| Kocide ® 3000 | 1.5 | lb/A | 6.3 d |
| Kocide ® 3000 | 1.5 lb/A/1 lb/A | | 7.8 cd |
| (apps ACEG)/ | | | |
| AIP61892 (apps BDFH) | | | |
| Kocide ® 3000 | 1.5 lb/A/2 lb/A | | 5.5 d |
| (apps ACEG)/ | | | |
| AIP61892 (apps BDFH) | | | |

### Example 8: AIP61892 and AIP1620 Applications in Combination Against Rhizoctonia

A tomato greenhouse trial was performed to test the efficacy of AIP61892 and AIP1620 against the pathogen Rhizoctonia solani. AIP61892 and AIP1620 alone and in combination were compared to the Acadia™ fungicide (azoxystrobin; Atticus, LLC). The testing rate for solo AIP1620 was 5 pounds or 2.5 pounds formulated AIP1620 per acre, while the testing rate for solo AIP61892 was 3 pounds or 1.5 pounds formulated AIP61892 per acre. The AIP1620+AIP61892 mix contained 2.5 pounds of product per acre of formulated AIP1620 plus 1.5 pounds product per acre of formulated AIP61892 or 5 pounds of product per acre of formulated AIP1620 plus 3 pounds product per acre of formulated AIP61892. The rootball of each plant was dipped in inoculum of Rhizoctonia solani prior to transplanting. Six plants were planted per pot. Immediately after transplanting, a single drench application was applied. The number of living plants were counted and recorded for each treatment after a total of 29 days after transplanting. Data were analyzed using R and ARM software. Means were compared with a Least Significant Difference test at the p=0.05 level. Statistically significant differences between means are indicated by different letters following the mean value of each treatment in Table 14. Synergy of the treatments was determined using Colby's method: the Expected response, E=X+Y−(XY/100), where X is the percent pathogen control by biocontrol agent A; and Y is the percent pathogen control by biocontrol agent B. If the observed response is greater than the expected response E, the combination is synergistic; if the observed response is less than the expected response E, the combination is antagonistic; and if the observed response is the same as the expected response E, the combination is additive.

Table 14 demonstrates that AIP1620 and AIP61892 alone have good activity on Rhizoctonia. An additive response was observed with the combination of AIP1620 5 lb/A and AIP61892 3 lb/A (100=100). An additive response was also observed with the combination of AIP1620 2.5 lb/A and AIP61892 1.5 lb/A (97=97).

TABLE 14
Efficacy of AIP61892 and AIP1620 on Rhizoctonia solani
in a Greenhouse Trial

| | | | Average Number of |
|---|---|---|---|
| | | | Live Plants (29 Days |
| Treatment | Dose Rate | | After Transplant) |
| AIP61892 | 3 | lb/A | 6 a |
| AIP61892 | 1.5 | lb/A | 5.8 a |
| AIP1620 | 5 | lb/A | 5.3 a |
| AIP1620 | 2.5 | lb/A | 4 b |
| Acadia ™ | 6 | oz | 5 a |
| AIP61892 + AIP1620 | 1.5 lb/A + 2.5 lb/A | | 5.8 a |
| AIP61892 + AIP1620 | 3 lb/A + 5 lb/A | | 6 a |
| Untreated Inoculated | — | | 2.3 c |
| Untreated Non- | — | | 6 a |
| Inoculated | | | |

### Example 9: AIP61892 and AIP1620 Applications in Combination Against Fusarium

A tomato greenhouse trial was performed to test the efficacy of AIP61892 and AIP1620 against the pathogen Fusarium oxysporum. AIP61892 and AIP1620 alone and in combination were compared to the Acadia™ fungicide (azoxystrobin; Atticus, LLC). The testing rate for solo AIP1620 was 5 pounds or 2.5 pounds formulated AIP1620 per acre, while the testing rate for solo AIP61892 was 3 pounds or 1.5 pounds formulated AIP61892 per acre. The AIP1620+AIP61892 mix contained 2.5 pounds of product per acre of formulated AIP1620 plus 1.5 pounds product per acre of formulated AIP61892 or 5 pounds of product per acre of formulated AIP1620 plus 3 pounds product per acre of formulated AIP61892. The rootball of each plant was dipped in inoculum of Fusarium oxysporum prior to transplanting. Six plants were planted per pot. Immediately after transplanting, a single drench application was applied. The number of living plants were counted and recorded for each treatment after a total of 29 days after transplanting. Data were analyzed using R and ARM software. Means were compared with a Least Significant Difference test at the p=0.05 level. Statistically significant differences between means are indicated by different letters following the mean value of each treatment in Table 15. Synergy calculations were determined using Colby's method as described in Example 8.

Table 15 demonstrates that AIP1620 and AIP61892 alone have activity on Fusarium. A synergistic response was observed with the combination of AIP1620 5 lb/A and AIP61892 3 lb/A (92>76). A synergistic response was also observed with the combination of AIP1620 2.5 lb/A and AIP61892 1.5 lb/A (67>63).

TABLE 15
Efficacy of AIP61892 and AIP1620 on Fusarium oxysporum
in a Greenhouse Trial

| | | | Average Number of |
|---|---|---|---|
| | | | Live Plants (29 Days |
| Treatment | Dose Rate | | After Transplant) |
| AIP61892 | 3 | lb/A | 3.3 bc |
| AIP61892 | 1.5 | lb/A | 3 bc |
| AIP1620 | 5 | lb/A | 2.8 bcd |
| AIP1620 | 2.5 | lb/A | 1.5 cd |
| Acadia ™ | 6 | oz | 1.5 cd |
| AIP61892 + AIP1620 | 1.5 lb/A + 2.5 lb/A | | 4 b |
| AIP61892 + AIP1620 | 3 lb/A + 5 lb/A | | 5.5 a |
| Untreated Inoculated | — | | 1.3 d |
| Untreated Non- | — | | 6 a |
| Inoculated | | | |

### Example 10: AIP61892 and AIP1620 Applications in Combination Against Phytophthora

A tomato greenhouse trial was performed to test the efficacy of AIP61892 and AIP1620 against the pathogen Phytophthora nicotianae. AIP61892 and AIP1620 alone and in combination were compared to the Ridomil Gold® fungicide (mefanoxam; Syngenta). The testing rate for solo AIP1620 was 5 pounds or 2.5 pounds formulated AIP1620 per acre, while the testing rate for solo AIP61892 was 3 pounds or 1.5 pounds formulated AIP61892 per acre. The AIP1620+AIP61892 mix contained 2.5 pounds of product per acre of formulated AIP1620 plus 1.5 pounds product per acre of formulated AIP61892 or 5 pounds of product per acre of formulated AIP1620 plus 3 pounds product per acre of formulated AIP61892. The rootball of each plant was dipped in inoculum of Phytophthora nicotianae prior to transplanting. Six plants were planted per pot. Immediately after transplanting, a single drench application was applied. The number of living plants were counted and recorded for each treatment after a total of 29 days after transplanting. Data were analyzed using R and ARM software. Means were compared with a Least Significant Difference test at the p=0.05 level. Statistically significant differences between means are indicated by different letters following the mean value of each treatment in Table 16. Synergy calculations were determined using Colby's method as described in Example 8.

Table 16 demonstrates that AIP1620 and AIP61892 alone have activity on Phytophthora. A synergistic response was observed with the combination of AIP1620 5 lb/A and AIP61892 3 lb/A (80>71). A synergistic response was also observed with the combination of AIP1620 2.5 lb/A and AIP61892 1.5 lb/A (67>56).

TABLE 16
Efficacy of AIP61892 and AIP1620 on Phytophthora nicotianae
in a Greenhouse Trial

| | | | Average Number of |
|---|---|---|---|
| | | | Live Plants (29 Days |
| Treatment | Dose Rate | | After Transplant) |
| AIP61892 | 3 | lb/A | 3.8 bc |
| AIP61892 | 1.5 | lb/A | 2.8 c |
| AIP1620 | 5 | lb/A | 1.3 d |
| AIP1620 | 2.5 | lb/A | 1 d |
| Ridomil Gold ® | 1 | pt | 3.5 bc |
| AIP61892 + AIP1620 | 1.5 lb/A + 2.5 lb/A | | 4 bc |
| AIP61892 + AIP1620 | 3 lb/A + 5 lb/A | | 4.8 b |
| Untreated Inoculated | — | | 0.5 d |
| Untreated Non- | — | | 6 a |
| Inoculated | | | |

### Example 8: AIP61892 and AIP1620 Applications in Combination Against Pythium

A tomato greenhouse trial was performed to test the efficacy of AIP61892 and AIP1620 against the pathogen Pythium ultimum. AIP61892 and AIP1620 alone and in combination were compared to the Ridomil Gold® fungicide (mefanoxam; Syngenta). The testing rate for solo AIP1620 was 5 pounds or 2.5 pounds formulated AIP1620 per acre, while the testing rate for solo AIP61892 was 3 pounds or 1.5 pounds formulated AIP61892 per acre. The AIP1620+AIP61892 mix contained 2.5 pounds of product per acre of formulated AIP1620 plus 1.5 pounds product per acre of formulated AIP61892 or 5 pounds of product per acre of formulated AIP1620 plus 3 pounds product per acre of formulated AIP61892. The rootball of each plant was dipped in inoculum of Pythium ultimum prior to transplanting. Six plants were planted per pot. Immediately after transplanting, a single drench application was applied. The number of living plants were counted and recorded for each treatment after a total of 29 days after transplanting. Data were analyzed using R and ARM software. Means were compared with a Least Significant Difference test at the p=0.05 level. Statistically significant differences between means are indicated by different letters following the mean value of each treatment in Table 17. Synergy calculations were determined using Colby's method as described in Example 8.

Table 17 demonstrates that AIP1620 and AIP61892 alone have activity on Pythium. A synergistic response was observed with the combination of AIP1620 5 lb/A and AIP61892 3 lb/A (88>74). A synergistic response was also observed with the combination of AIP1620 2.5 lb/A and AIP61892 1.5 lb/A (80>61).

TABLE 17
Efficacy of AIP61892 and AIP1620 on
Pythium ultimum in a Greenhouse Trial

| | | | Average Number of |
|---|---|---|---|
| | | | Live Plants (29 Days |
| Treatment | Dose Rate | | After Transplant) |
| AIP61892 | 3 | lb/A | 3.5 d |
| AIP61892 | 1.5 | lb/A | 3 de |
| AIP1620 | 5 | lb/A | 2.3 ef |
| AIP1620 | 2.5 | lb/A | 1.3 fg |
| Ridomil Gold ® | 1 | pt | 4 cd |
| AIP61892 + AIP1620 | 1.5 lb/A + 2.5 lb/A | | 4.8 bc |
| AIP61892 + AIP1620 | 3 lb/A + 5 lb/A | | 5.3 ab |
| Untreated Inoculated | — | | 0.5 g |
| Untreated Non- | — | | 6 a |
| Inoculated | | | |

### Example 9: AIP61892 and AIP1620 Applications Against Pathogens in Post-Harvested Cherries

AIP61892 and AIP1620 were evaluated for control of several important post-harvest diseases, including those induced by the genera Botrytis, Monilinia, and Rhizopus.

### Example 9.1: AIP1620 Effective Against Monilinia and Botrytis on Sweet Cherries

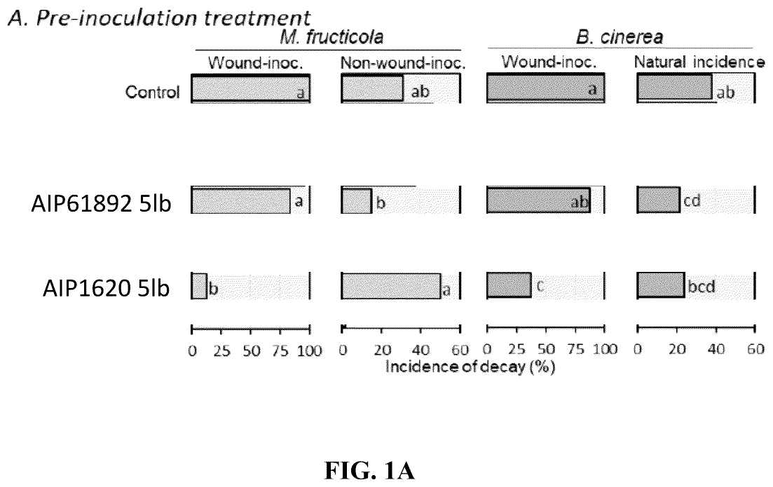

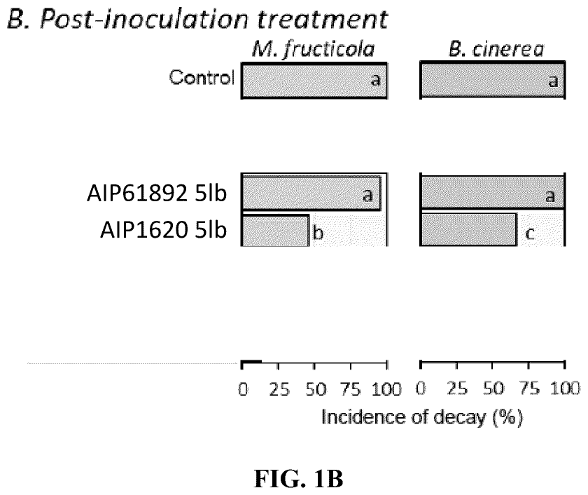

Monilinia fructicola is the causative agent for Brown Rot and Botrytis cinerea is the causative agent for Gray Mold on sweet cherries. Harvested sweet cherry fruit were spray-treated with AIP1620 or AIP61892 prior to wound or non-wound inoculation (FIG. 1A) with 50,000 spores/ml of M. fructicola or wound inoculated with 300,000 spores/ml of B. cinerea. Gray mold was also allowed to develop naturally. When treated prior to wound inoculation, AIP1620 was effective and significantly reduced decay caused by both brown rot and gray mold as compared to the untreated control. For non-wound inoculated fruit, AIP61892 significantly reduced brown rot, whereas AIP61892 and AIP1620 both significantly reduced the natural incidence of gray mold.

In other experiments (FIG. 1B), harvested sweet cherry fruit were wound-inoculated with 30,000 spores/ml of M. fructicola or B. cinerea and incubated for 8 h at 20° C. Aqueous treatments or AIP1620 or AIP61892 were applied using an air-nozzle sprayer, and fruit were incubated at 20° C. for 6-8 days. When the biologicals were used as post-inoculation treatments, only AIP1620 reduced brown rot and gray mold.

### Example 9.2: AIP1620 Effective Against Monilinia, Botrytis, and Rhizopus on Bing Cherries

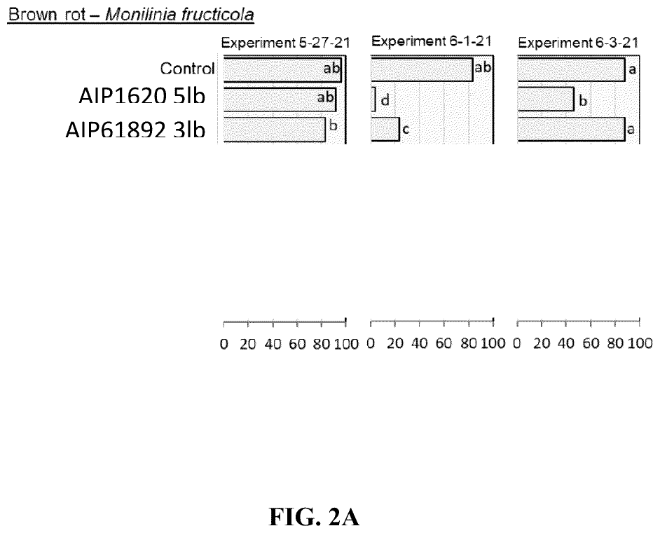

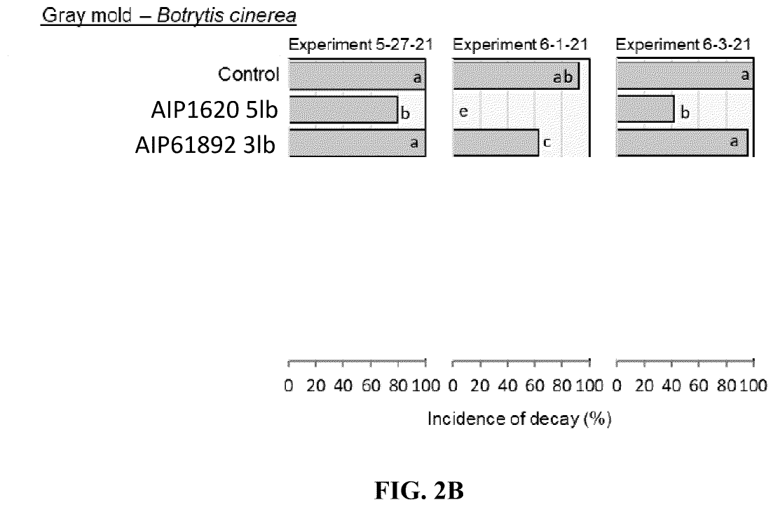

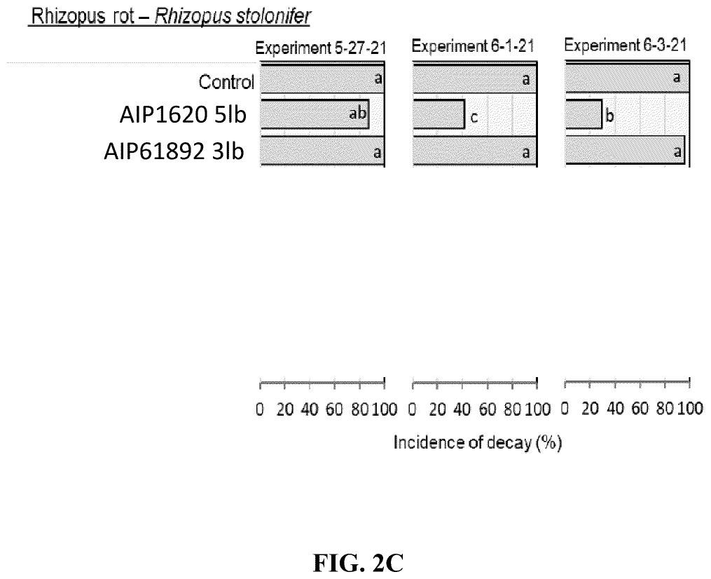

Monilinia fructicola is the causative agent for Brown Rot, Botrytis cinerea is the causative agent for Gray Mold, and Rhizopus stolonifera is the causative agent for Rhizopus rot on Bing cherries. In these studies, harvested fruit were wound-inoculated with spores of M. fructicola (50,000 spores/ml), B. cinerea (20,000 spores/ml), or R. stolonifera (20,000 spores/ml) and incubated for 12 h at 20° C. Aqueous treatments of AIP1620 or AIP61892 were applied using an air-nozzle sprayer. Fruit were incubated at 20° C. for 4-7 days before evaluation. As can be seen in FIG. 2A-2C, AIP1620 and AIP61892 were effective in some trials, but not in others, against brown rot (FIG. 2A) and only AIP1620 was effective against gray mold (FIG. 2B) and Rhizopus rot (FIG. 2C).

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A stable formulation comprising a biocontrol agent, wherein the biocontrol agent comprises:
(a) a bacterial strain deposited as NRRL No. B-67089; or
(b) a supernatant, fermentation product, filtrate, or extract derived from a whole cell culture of a bacterial strain deposited as NRRL No. B-67089.

2. The stable formulation of claim 1, wherein the formulation is a dry formulation or a liquid formulation.

3. The stable formulation of claim 1 or 2, wherein the biocontrol agent is present at about 10⁵ CFU/gram to about 10¹² CFU/gram or at about 10⁵ CFU/ml to about 10¹² CFU/ml.

4. The stable formulation of any one of claims 1-3, wherein the formulation further comprises a pesticide, a fungicide, a bactericide, a nematicide, an insecticide, or an herbicide.

5. The stable formulation of claim 4, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

6. The stable formulation of any one of claims 1-5, wherein the formulation comprises a second biocontrol agent.

7. The stable formulation of claim 6, wherein the second biocontrol agent comprises Bacillus amyloliquefaciens strain D747, Bacillus subtilis strain QST 713, Bacillus amyloliquefaciens strain GB03, Bacillus subtilis strain F1B17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

8. The stable formulation of claim 7, wherein Pseudomonas fluorescens strain AIP1620 is present at about 10⁵ CFU/gram to about 10¹² CFU/gram, at about 10⁷ total cells/gram to about 10¹⁴ total cells/gram, at about 10⁵ CFU/ml to about 10¹² CFU/ml, or at about 10⁷ total cells/ml to about 10¹⁴ total cells/ml.

9. A coated seed comprising a seed and a coating on the seed, wherein the coating comprises a formulation comprising a biocontrol agent, wherein the biocontrol agent comprises a bacterial strain deposited as NRRL No. B-67089.

10. The coated seed of claim 9, wherein the biocontrol agent is present at about 10⁵ CFU/gram to about 10¹² CFU/gram or at about 10⁵ CFU/ml to about 10¹² CFU/ml.

11. The coated seed of claim 9 or 10, wherein the coating further comprises a pesticide, a biocide, a fungicide, a bactericide, a nematicide, an insecticide or an herbicide.

12. The coated seed of claim 11, wherein the fungicide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

13. The coated seed of any one of claims 9-12, wherein the coating comprises a second biocontrol agent.

14. The coated seed of claim 13, wherein the second biocontrol agent comprises Bacillus amyloliquefaciens strain D747, Bacillus subtilis strain QST 713, Bacillus amyloliquefaciens strain GB03, Bacillus subtilis strain F1B17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

15. The coated seed of claim 14, wherein Pseudomonas fluorescens strain AIP1620 is present at about 10⁵ CFU/gram to about 10¹² CFU/gram, at about 10⁷ total cells/gram to about 10¹⁴ total cells/gram, at about 10⁵ CFU/ml to about 10¹² CFU/ml, or at about 10⁷ total cells/ml to about 10¹⁴ total cells/ml.

16. A composition comprising an effective amount of a biocontrol agent, wherein the biocontrol agent comprises:
(a) a bacterial strain deposited as NRRL No. B-67089; or
(b) a supernatant, fermentation product, filtrate, or extract derived from a whole cell culture of a bacterial strain deposited as NRRL No. B-67089;
wherein said effective amount of said composition controls a plant pest and/or improves at least one agronomic trait of interest and/or improves plant health.

17. The composition of claim 16, wherein the bacterial strain is present in about 10⁵ CFU/gram to about 10¹² CFU/gram or at about 10⁵ CFU/ml to about 10¹² CFU/ml.

18. The composition of claim 16 or 17, wherein the composition further comprises a pesticide, a biocide, a fungicide, a bactericide, a nematicide, an insecticide, or an herbicide.

19. The composition of claim 18, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

20. The composition of any one of claims 16-19, wherein the composition comprises a second biocontrol agent.

21. The composition of claim 20, wherein the second biocontrol agent comprises Bacillus amyloliquefaciens strain D747, Bacillus subtilis strain QST 713, Bacillus amyloliquefaciens strain GB03, Bacillus subtilis strain F1B17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

22. The composition of claim 21, wherein Pseudomonas fluorescens strain AIP1620 is present at about 10⁵ CFU/gram to about 10¹² CFU/gram, at about 10⁷ total cells/gram to about 10¹⁴ total cells/gram, at about 10⁵ CFU/ml to about 10¹² CFU/ml, or at about 10⁷ total cells/ml to about 10¹⁴ total cells/ml.

23. A method for controlling a plant pest comprising contacting said pest with an effective amount of the composition of any one of claims 16-22, or the formulation of any one of claims 1-8, wherein said composition or said formulation controls said plant pest.

24. A method for growing a plant susceptible to a plant pest comprising applying to a plant, a plant part, a seed, or an area of cultivation a biocontrol agent, wherein the biocontrol agent comprises:
(a) an effective amount of a bacterial strain deposited as NRRL No. B-67089; or
(b) an effective amount of a supernatant, fermentation product, filtrate, or extract derived from a whole cell culture of a bacterial strain deposited as NRRL No. B-67089;
wherein said effective amount controls a plant pest.

25. A method of controlling a plant pest comprising applying to a plant, a plant part, a seed, or an area of cultivation a biocontrol agent, wherein the biocontrol agent comprises:
(a) an effective amount of a bacterial strain deposited as NRRL No. B-67089; or
(b) an effective amount of a supernatant, fermentation product, filtrate, or extract derived from a whole cell culture of a bacterial strain deposited as NRRL No. B-67089;
wherein said effective amount controls the plant pest.

26. The method of claim 24 or 25, wherein the biocontrol agent is applied to the plant or plant part prior to harvest.

27. The method of claim 24 or 25, wherein the biocontrol agent is applied to the plant or plant part after harvest.

28. The method of claim 27, wherein the biocontrol agent is applied by dipping, drenching, flooding, fogging, spraying, dusting, or injecting.

29. The method of any one of claims 23-28, wherein said effective amount of the bacterial strain comprises at least about 10⁴ to 10¹⁶ colony forming units (CFU) per hectare.

30. The method of any one of claims 23-29, wherein the plant pest is an insect, nematode, fungus, fungal-like organism, virus, viroid, bacterium, or a protozoan pathogen.

31. The method of any one of claims 23-29, wherein said plant pest comprises one or more bacterial pathogens selected from the group consisting of Acidovorax avenae, Burkholderia gladioli, Candidatus Liberibacter spp., Clavibacter michiganensis, Erwinia amylovora, Erwinia ananas, Erwinia chrysanthemi, Erwinia dissolvens, Erwinia herbicola, Erwinia rhapontic, Erwinia stewartii, Erwinia tracheiphila, Erwinia uredovora; Pectobacterium carotovorum, Pseudomonas syringae, Streptomyces scabies, Xanthomonas campestris, Xanthomonas axonopodis, Xanthomonas fragariae; Xanthomonas translucens, and Xylella fastidiosa.

32. The method of any one of claims 23-29, wherein said bacterial pathogen is Erwinia spp., Pseudomonas spp., or Xanthomonas spp.

33. The method of any one of claims 23-29, wherein said plant pest comprises one or more selected from the group consisting of Aspergillus spp., Botrytis spp., Cercospora spp., Alternaria spp., Didymella spp., Fusarium spp., Erysiphe spp., Colletotrichum spp., Monilinia spp., Mycosphaerella spp., Plasmopara spp., Peronospora spp., Pythium spp., Phytophthora spp., Phomopsis spp., Phakopsora spp., Podosphaera spp., Rhizopus spp., Rhizoctonia spp., Sclerotium spp., Sclerotinia spp., Uncinula spp., Venturia spp., Wilsonomyces spp., and Plasmodiophora spp.

34. The method of any one of claims 23-29, wherein said plant pest comprises one or more selected from the group consisting of Aspergillus flavus, Botrytis cinerea, Cercospora sojina, Alternaria solani, Colletotrichum acutatum, Colletotrichum cereal, Colletotrichum sublineolum, Didymella bryoniae, Erysiphe necator, Fusarium graminearum, Fusarium solani, Fusarium oxysporum, Monilinia fructicola, Monilinia laxa, Monilinia fructigena, Mycosphaerella citri, Mycosphaerella fijiensis, Podosphaera xanthii, Plasmopara viticola, Plasmodiophora brassicae, Peronospora belbahrii, Pythium aphanidermatum, Pythium sylvaticum, Pythium myriotylum, Pythium ultimum, Phytophthora nicotianae, Phytophthora infestans, Phytophthora tropicalis, Phytophthora sojae, Phakopsora pachyrizi, Rhizoctonia solani, Rhizopus stolonifera, Sclerotium rolfsii, Sclerotinia minor, Sclerotinia sclerotiorium, Uncinula necator, and Venturia inaequalis.

35. The method of any one of claims 23-34, wherein the method further comprises applying an effective amount of a pesticide, a fungicide, a bactericide, a nematicide, an insecticide, or an herbicide.

36. The method of claim 35, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

37. The method of any one of claims 23-36, wherein the method further comprises applying an effective amount of a second biocontrol agent.

38. The method of claim 37, wherein the second biocontrol agent comprises Bacillus amyloliquefaciens strain D747, Bacillus subtilis strain QST 713, Bacillus amyloliquefaciens strain GB03, Bacillus subtilis strain F1B17, Bacillus pumilus strain QST 2808, Bacillus pumilus strain GB34, Bacillus subtilis var. amyloliquefaciens strain FZ1B24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

39. The method of claim 38, wherein the effective amount of Pseudomonas fluorescens strain AIP1620 comprises at least about 10⁴ to 10¹⁶ colony forming units (CFU) per hectare or at least about 10⁶ to 10¹⁸ total cells per hectare.

40. The method of any one of claims 37-39, wherein the biocontrol agent and the second biocontrol agent are applied simultaneously.

41. The method of any one of claims 37-39, wherein the biocontrol agent and the second biocontrol agent are applied sequentially.

42. A method of treating or preventing a plant disease comprising applying to a plant or plant part a plant, a plant part, a seed, or an area of cultivation a biocontrol agent, wherein the biocontrol agent comprises:
(a) an effective amount of a biocontrol agent comprising a bacterial strain deposited as NRRL No. B-67089; or
(b) an effective amount of a supernatant, fermentation product, filtrate, or extract derived from a whole cell culture of a bacterial strain deposited as NRRL No. B-67089;
wherein the effective amount treats or prevents the plant disease.

43. The method of claim 42, wherein said effective amount of the bacterial strain comprises at least about 10⁴ to 10¹⁶ CFU per hectare.

44. The method of claim 42 or 43, wherein the plant disease is Asian Soybean Rust (ASR), gray mold, leaf spot, Frogeye Leaf Spot, Early Blight, Damping off complex, Brown Patch, black scurf, root rot, belly rot, Sheath Blight, Powdery Mildew, Anthracnose, Black Sigatoka, Anthracnose leaf spot, Downy Mildew, Pythium Blight, Late Blight, Fusarium Head Blight, sudden death syndrome (SDS), Fusarium Wilt, Corn Stalk Rot, Brown Rust, Black Rust, Yellow Rust, Wheat Rust, Rust, Apple Scab, Post-bloom Fruit Drop, Gummy Stem Blight, Greasy Spot, Corn Stalk Rot, Cherry Blossom Blight, Damping Off, Fire Blight, Citrus Greening Disease, Clubroot, Verticillium Wilt, Rhizopus Rot, Bacterial Spot, or Brown Rot.

45. The method of any one of claims 42-44, wherein the method further comprises applying an effective amount of a pesticide, a fungicide, a bactericide, a nematicide, an insecticide, or an herbicide.

46. The method of claim 45, wherein the fungicide or bactericide comprises prothioconazole, azoxystrobin, fluopicolide, chlorothalonil, fosetyl, fenhexamid, flutriafol, difenoconazole, tebuconazole, tetraconazole, pyraclostrobin, trifloxystrobin, propiconazole, fluoxastrobin, flutolanil, metconazole, a copper compound, or metrafenone.

47. The method of any one of claims 42-46, wherein a second biocontrol agent is applied at an effective amount.

48. The method of claim 47, wherein the second biocontrol agent comprises Bacillus amyloliquefaciens strain D747, Bacillus subtilis strain QST 713, Bacillus amyloliquefaciens strain GB03, Bacillus subtilis strain FB17, Bacillus pumilus strain QST 2808, Bacillus pumilus strain GB34, Bacillus subtilis var. amyloliquefaciens strain FZB24, Bacillus finnus strain CNMC 1-1582, Streptomyces lydicus strain WYEC108, Streptomyces griseoviridis strain K61, Agrobacterium radiobacter strain 1026, Agrobacterium radiobacter strain K84, Pseudomonas fluorescens strain A506, Bacillus thuringiensis subspecies aizawai strain GC-91, Bacillus thuringiensis subspecies kurstaki, Bacillus thuringiensis subspecies kurstaki strain EG7841, Bacillus thuringiensis subspecies kurstaki strain SA-12, Bacillus thuringiensis subspecies kurstaki strain ABTS-351, Bacillus thuringiensis subspecies kurstaki strain SA-11, Bacillus thuringiensis subspecies tenebrionis strain SA-10, Chromobacterium subtsugae strain PRAA4-1, Isaria fumosorosea Apopka Strain 97, Burkholderia spp. strain A396, or Bacillus thuringiensis subspecies aizawai strain ABTS-1857, or Pseudomonas fluorescens strains AIP050999 or AIP1620.

49. The method of claim 48, wherein the effective amount of Pseudomonas fluorescens strain AIP1620 comprises at least about 10⁴ to 10¹⁶ colony forming units (CFU) per hectare or at least about 10⁶ to 10¹⁸ total cells per hectare.

50. The method of any one of claims 47-49, wherein the biocontrol agent and the second biocontrol agent are applied simultaneously.

51. The method of any one of claims 47-49, wherein the biocontrol agent and the second biocontrol agent are applied sequentially.

52. The method of any one of claims 42-51, wherein the biocontrol agent is applied to the plant or plant part after harvest.

53. The method of claim 52, wherein the biocontrol agent is applied by dipping, drenching, flooding, fogging, spraying, dusting, or injecting.

54. A method for improving at least one agronomic trait of interest in a plant and/or improving the health of a plant comprising applying to said plant an effective amount of the composition of any one of claims 16-22, or the formulation of any one of claims 1-8, wherein said composition or said formulation improves at least one agronomic trait of interest of said plant and/or improves the health of said plant when compared to a plant to which the composition or formulation was not applied.

55. A method for improving at least one agronomic trait of interest in a plant and/or improving the health of a plant comprising applying to a plant, a plant part, a seed, or an area of cultivation a biocontrol agent, wherein the biocontrol agent comprises:
(a) an effective amount of a bacterial strain deposited as NRRL No. B-67089; or
(b) an effective amount of a supernatant, fermentation product, filtrate, or extract derived from a whole cell culture of a bacterial strain deposited as NRRL No. B-67089;
wherein said effective amount improves at least one agronomic trait of interest of said plant and/or improves the health of said plant when compared to a plant to which the biocontrol agent was not applied.

56. The method of claim 55, wherein said effective amount of the bacterial strain comprises at least about 10⁴ to 10¹⁶ colony forming units (CFU) per hectare.

57. The method of claim 55 or 56, wherein a second biocontrol agent is applied at an effective amount.

58. The method of claim 57, wherein the second biocontrol agent comprises Pseudomonas fluorescens strain AIP1620.

59. The method of claim 58, wherein the effective amount of Pseudomonas fluorescens strain AIP1620 comprises at least about 10⁴ to 10¹⁶ colony forming units (CFU) per hectare or at least about 10⁶ to 10¹⁸ total cells per hectare.

60. The method of any one of claims 57-59, wherein the biocontrol agent and the second biocontrol agent are applied simultaneously.

61. The method of any one of claims 57-59, wherein the biocontrol agent and the second biocontrol agent are applied sequentially.

62. The method of any one of embodiments 37-41, wherein control of said plant pest is increased synergistically.

63. The method of embodiment 62, wherein said plant pest is selected from:
a. Rhizoctonia, such as Rhizoctonia solani;
b. Fusarium, such as Fusarium oxysporum;
c. Phytophthora, such as Phytophthora nicotianae; and
d. Phythium, such as Pythium ultimum.
